(19) United States
(12) Patent Application Publication
(10) Pub. No.: US 2026/0207775 A1
(43) Pub. Date: Jul. 23, 2026

(54) ANTISENSE OLIGONUCLEOTIDES FOR THE TREATMENT OF CARDIOVASCULAR DISEASE

(71) Applicant: ProQR Therapeutics II B.V., Leiden (NL)

(72) Inventors: Marko POTMAN, Leiden (NL); Aliye Seda YILMAZ-ELIS, Leiden (NL); Bart KLEIN, Leiden (NL); Petra Geziena DE BRUIJN, Leiden (NL); Bruno Filipe Madeira DE ALBUQUERQUE, Leiden (NL); Angela HELFRICHT, Leiden (NL); Peter Christian DE VISSER, Leiden (NL)

(73) Assignee: ProQR Therapeutics II B.V., Leiden (NL)

(21) Appl. No.: 19/137,225

(22) PCT Filed: Dec. 8, 2023
(86) PCT No.: PCT/EP2023/084865

§ 371 Date: Jun. 9, 2025

(30) Foreign Application Priority Data
Dec. 9, 2022 (GB) 2218585.4
May 8, 2023 (GB) 2306756.4

(51) Int. Cl. A61K 48/00; A61P 7/00; C12N 9/10; C12N 9/78; C12N 15/11; C12N 15/86; C12N 15/88; C12Q 1/6869; C12Q 1/6883

(52) U.S. Cl. CPC A61K 48/005; A61P 7/00; C12N 9/1051; C12N 9/78; C12N 15/111; C12N 15/86; C12N 15/88; C12Q 1/6869; C12Q 1/6883; C12N 2310/20; C12N 2310/322; C12N 2750/14143; C12Q 2600/106; C12Y 204/01274; C12Y 305/04004

(57) ABSTRACT

The invention relates to the field of diseases caused by high levels of LDL-C and/or fibrinogen, such as cardiovascular disease. The invention involves oligonucleotides for RNA editing technology in deaminating target adenosine nucleotides, such as the adenosine at position 1055, in transcripts of the human B4GALT1 gene.

### TECHNICAL FIELD

This invention relates to the field of medicine and in particular cardiovascular disease (CVD). The invention involves the use of RNA editing technology in targeting the transcript encoding the enzyme β-1,4-galactosyl transferase 1 (B4GALT1) to bring about amino acid changes that reduce B4GALT1 function.

### BACKGROUND

CVD is one of the leading causes of death and disability worldwide. The provision of therapies for CVD therefore represents a significant unmet medical need. A variety of risk factors exists for the development of CVD. These risk factors include elevated blood levels of low-density lipoprotein cholesterol (LDL-C) and/or elevated levels of fibrinogen (Montasser et al. 2021. Science 374:1221-1227). Increased LDL-C concentration rises the likelihood of arterial plaque formation. Atherosclerosis and fibrinogen increase the risk of blood clotting and thrombosis. LDL-C is an established risk factor for coronary artery disease (CAD). The identification of mechanisms by which to reduce the blood levels of LDL-C and/or fibrinogen would therefore provide potential targets by which to prevent, ameliorate, or treat CVD.

A recently identified target in the fight against CVD is the enzyme B4GALT1, that is involved in the processing of biologically important biomolecules, including those that are involved in lipid metabolism and coagulation. B4GALT1 is ubiquitously expressed and plays a critical role in the processing of N-linked oligosaccharide moieties in glycoproteins, transferring the galactose from uridine diphosphate galactose (UDP-Gal) to specific glycoprotein substrates. B4GALT1 is therefore important for biological activity associated with correctly constructed oligosaccharides.

Recent research identified an association between a B4GALT1 mutation that exists within the Old Order Amish population and a reduction in CVD in that population (Montasser et al. 2021, supra). Importantly, B4GALT1 gene-based analysis in nearly 600,000 subjects revealed an association with decreased CAD. This population was found to have an enriched missense variant, referred to as p.Asn352Ser (asparagine to serine; also herein referred to as p.N352S, or simply as N352S) in the B4GALT1 protein, and decreased blood concentrations of LDL-C and fibrinogen were associated with this variant. The effect of this alteration was systematically analysed through a variety of techniques. Knock-in mouse studies provided evidence that the N352S variation has a causative role in decreased LDL-C and fibrinogen levels in blood. N-linked glycan profiling of serum from human subjects with this mutation was associated with decreased galactosylation and sialylation of apolipoprotein B100, fibrinogen, immunoglobulin G, and transferrin. Enzymatic assays revealed that this mutation results in a 50% lower galactosyltransferase activity compared with the non-mutated protein. Structural studies showed position 352 of B4GALT1 is in a functional domain of B4GALT1 and mutation from asparagine to serine was understood to hinder the conformational change required for enzymatic activity, affecting the glycosylation efficiency of B4GALT1.

The B4GALT1 N352S mutation was therefore observed to affect at least two separate risk factors associated with CVD, namely reduction of blood LDL-C and fibrinogen concentrations, and therefore may be one of the first targets to have pleiotropic ability to protect against CVD. Importantly, the B4GALT1 N352S missense variant was not seen to be associated with any severe phenotype.

Targeted modulation of galactosylation has been identified as a potential therapeutic target for preventing, treating, or ameliorating CVD while avoiding significant side effects, though no mechanism by which to modulate galactosylation was proposed. The present disclosure aims to provide one or more alternative and/or improved techniques, compounds and/or compositions for use in the treatment of CVD.

### SUMMARY OF THE INVENTION

The present disclosure provides an RNA editing oligonucleotide (EON) that can form a double-stranded complex with a region of a target RNA nucleic acid molecule in a human cell, wherein the double-stranded complex can recruit an endogenous ADAR enzyme naturally present in the cell, wherein the region comprises a target adenosine, wherein the nucleotide in the EON that is opposite the target adenosine is the orphan nucleotide, wherein the ADAR enzyme deaminates the target adenosine into an inosine, and wherein the target RNA nucleic acid molecule is a transcript molecule of the human β-1,4-galactosyl-transferase 1 (B4GALT1) gene. In a preferred aspect, the B4GALT1 transcript molecule is a pre-mRNA or an mRNA molecule. In another preferred aspect, the cell is a human liver cell, preferably a hepatocyte.

In one aspect, the target adenosine in the B4GALT1 transcript molecule is at a position where a guanosine would encode a B4GALT1 protein variant that has a reduced enzymatic turnover rate. In a preferred aspect, the target adenosine is at position c. 1055A in the B4GALT1 transcript, and wherein the deamination results in a change from asparagine (N; Asn; encoded by an AAU codon) to serine (S; Ser; encoded by an AGU codon) at position 352 in the human wildtype B4GALT1 amino acid sequence.

The disclosure provides an EON as disclosed herein, wherein at least one nucleotide comprises one or more non-naturally occurring chemical modifications in the ribose, linkage, or base moiety, with the proviso that the orphan nucleotide is not a cytidine comprising a 2′-OMe ribose substitution. In a preferred aspect, the one or more modifications in the linkage is each independently selected from a phosphorothioate (PS), a phosphonoacetate, phosphorodithioate, a methylphosphonate (MP), a sulfonylphosphoramidate, or a PNdmi internucleoside linkage. In a preferred aspect, the one or more modifications in the ribose moiety is a mono- or di-substitution at the 2′, 3′ and/or 5′ position of the ribose, each independently selected from the group consisting of: —OH; —F; substituted or unsubstituted, linear or branched lower (C₁-C₁₀) alkyl, alkenyl, alkynyl, alkaryl, allyl, or aralkyl, that may be interrupted by one or more heteroatoms; —O-, S-, or N-alkyl; —O-, S-, or N-alkenyl; —O-, S-, or N-alkynyl; —O-, S-, or N-allyl; —O-alkyl-O-alkyl; -methoxy; -aminopropoxy; -methoxyethoxy; -dimethylamino oxyethoxy; and -dimethylaminoethoxyethoxy.

Provided herein is an EON, wherein the EON comprises or consists of an EON selected from the group provided in SEQ ID NO:3 to 42, 59 to 1069, and 1078 to 1190, preferably from the group consisting of SEQ ID NO:23, 19, 31, 27, 35, 39, 69, 70, 71, 72, 73, 93, 94, 95, 1079, 1084, 1093, 1095, 1100, 1102, 1115, 1121, 1123, 1124, and 1139 to 1190. Importantly, provided herein is an EON that comprises a base nucleotide sequence according to these preferred EONs that are further modified as outlined herein, which means that these preferred EONs may even be further optimized to reach an even more efficient RNA editing effect, using the teaching provided herein. In a preferred aspect, the orphan nucleotide in the EON as disclosed herein is a deoxynucleotide carrying a 6-amino-5-nitro-3-yl-2 (1H)-pyridone nucleobase (Benner's base Z) or an iso-uracil nucleobase (isoU).

Provided herein is also a vector, preferably a viral vector, more preferably an adeno-associated virus (AAV) vector, comprising a nucleic acid molecule encoding an EON as disclosed herein. Provided herein is also a nanoparticle delivery vehicle formulation that comprises an EON as disclosed herein. In a preferred aspect, the nanoparticle delivery vehicle is a Lipid Nanoparticle (LNP). Provided herein is also a pharmaceutical composition comprising an EON, a vector, or a nanoparticle delivery vehicle formulation, as disclosed herein, and a pharmaceutically acceptable carrier.

In one aspect, the disclosure provides an EON, a vector, a nanoparticle delivery vehicle formulation, or a pharmaceutical composition, as disclosed herein, for use in the treatment of CVD.

In one aspect, the disclosure provides a use of an EON, a vector, a nanoparticle delivery vehicle formulation, or a pharmaceutical composition, as disclosed herein, in the manufacture of a medicament for the treatment of CVD.

In one aspect, the disclosure provides an in vitro, ex vivo, or in vivo method of editing a B4GALT1 transcript molecule, the method comprising contacting the B4GALT1 transcript molecule, or a part thereof, with an EON as disclosed herein, thereby allowing the formation of a double-stranded complex of the EON with the B4GALT1 transcript molecule, thereby enabling the recruitment of an ADAR1 or ADAR2 deamination enzyme that binds to the double-stranded complex, and therethrough allowing the specific editing of an adenosine in the B4GALT1 transcript molecule, by the deamination enzyme, into an inosine.

In one aspect, the disclosure provides a method of treating, slowing down, or ameliorating CVD in a patient in need thereof, the method comprising contacting a B4GALT1 transcript molecule in a cell of the subject with an EON as disclosed herein, thereby treating the patient.

In one aspect, the disclosure provides a method for the deamination of a target adenosine in an B4GALT1 transcript molecule in a cell, the method comprising the steps of: (i) providing the cell with an EON, a vector, or a nanoparticle delivery vehicle formulation, as disclosed herein; (ii) allowing uptake by the cell of the EON, the vector, or the nanoparticle delivery vehicle; (iii) allowing annealing of the EON to the B4GALT1 transcript molecule; (iv) allowing an endogenous ADAR enzyme that is naturally present in the cell to deaminate the target adenosine in the B4GALT1 transcript molecule to an inosine; and optionally (v) identifying the presence of the inosine in the target RNA molecule. In a preferred aspect, the cell is a human cell, preferably a liver cell, more preferably a hepatocyte, wherein the target adenosine is at position c. 1055A in the B4GALT1 transcript, and wherein the deamination results in a change from asparagine (N; Asn) to serine (S; Ser) at position 352 in the human wildtype B4GALT1 amino acid sequence. In a preferred aspect, step (v) of the method as disclosed herein comprises: a) sequencing the B4GALT1 pre-mRNA or mRNA molecule, or a cDNA derived thereof; b) assessing the presence of a 352Ser B4GALT1 protein variant; or c) using a functional read-out, preferably assessing a reduction rate of UDP-Gal, or assessing glycosylation levels of transferrin in serum.

### BRIEF DESCRIPTION OF THE DRAWINGS

One or aspects will now be described, by way of example only, with reference to the accompanying drawings:

FIG. 1A shows a nucleotide sequence of a wildtype human B4GALT1 RNA transcript from start codon to stop codon, showing the start codon and stop codon, wherein the target adenosine at nucleotide position 1055 (in the AAU codon encoding asparagine at amino acid position 352) is shown in bold font. FIG. 1B shows an amino acid sequence of a human wildtype B4GALT1 protein, wherein the asparagine at amino acid position 352 (N) is shown in bold font.

FIG. 2 shows sequences (5′ to 3′) of EONs designed for editing the B4GALT1 transcript, with their respective SEQ ID NO's as shown. RM4838/EON13 is also referred to as B4GALT1-13. RM4830/EON05 is also referred to as B4GALT1-05. The chemical modifications of the EONs are as follows: m5Ue is 2′-MOE modified 5-methyl-uridine (similar to a 2′-MOE modified thymidine); m5Ce is 2′-MOE modified 5-methyl-cytidine; Ae and Ge are 2′-MOE modified adenosine and guanosine, respectively; Gm, Am, Um, and Cm are 2′-OMe modified guanosine, adenosine, uridine, and cytidine, respectively; Af, Uf, Gf, and Cf are 2′-F modified adenosine, uridine, guanosine, and cytosine, respectively; Zd (at the orphan nucleotide position) is a cytidine analog that is also referred to as a nucleoside carrying a Benner's base (as further outlined herein), with a deoxy moiety (=DNA) at the 2′ ribose position; C2f (at the orphan nucleotide position) is a 2′,2′-difluoro modified cytidine; m5Ud (or simply referred to as Ud) is a deoxynucleotide with a 5-methyl-uridine base; Cd (at the orphan nucleotide position) is deoxycytidine; an asterisk * refers to a PS linkage; a “!” refers to a PNdmi linkage; and a “\hat{ }” refers to a MP linkage. All other linkages are phosphodiester linkages.

FIG. 3 shows the percentage editing of endogenous B4GALT1 transcripts in human HepG2 cells after treatment with the indicated EONs in two different concentrations (1 and 5 μM) and saponin (AG1856). Negative controls were AG1856 only and non-treated cells.

FIG. 4 shows the percentage exon 5 skip in endogenous B4GALT1 pre-mRNA in human HepG2 cells after treatment with the indicated EONs in two different concentrations (1 and 5 μM) and saponin (AG1856). Negative controls were AG1856 only and non-treated cells.

FIG. 5 shows (A) the percentage editing of endogenous B4GALT1 transcripts in liver spheroids generated from primary human hepatocytes, after treatment with the four indicated EONs, and (B) the percentage exon 5 skipping in these same samples. Negative controls were saponin alone (NT+AG) and non-treated cells.

FIG. 6 shows a set of EONs (SEQ ID NO:62 to 108, as indicated) designed with the addition of a GalNAc moiety attached to the 5′ terminus of the oligonucleotide. The EON of SEQ ID NO: 1069 (B4GALT1-134(−)) is the same as the EON of SEQ ID NO:72 (B4GALT1-134), but without the GalNAc moiety and the linker between the GalNAc and the oligonucleotide. The chemical modifications are as provided in FIG. 2. L001 is a tri-antennary GalNAc moiety (OP-042; Hongene Biotech). L103 is a TEG linker linking the GalNAc moiety to the first nucleotide on the 5′ terminus.

FIG. 7 (A) shows the editing percentage of the human B4GALT1 target transcript in primary human hepatocytes (PHH) after treatment with 5 μM EON in the presence of 1 μM saponin (AG1856), using the EONs of FIG. 6 together with EON01 and EON05 (see FIG. 2). A non-treated (NT) sample was taken as negative control. (B) shows the percentage of exon 5 skip observed in the same samples.

FIG. 8(A) shows the editing percentage of the human B4GALT1 target transcript in PHH after treatment with 5 μM EON in the absence of saponin, hence through gymnotic uptake, using the EONs of FIG. 6 together with EON01 and EON05 (see FIG. 2). A non-treated (NT) sample was taken as negative control. (B) shows the percentage of exon 5 skip observed in the same samples.

FIG. 9(A) shows the editing percentage of the B4GALT1 target transcript in primary mouse hepatocytes (PMH) after treatment with 5 μM EON in the absence of saponin, hence through gymnotic uptake, using the EONs of FIG. 6 together with EON01 and EON05 (see FIG. 2). A non-treated (NT) sample was taken as negative control. (B) shows the percentage of exon 5 skip observed in the same samples.

FIG. 10(A) shows the editing percentage of the B4GALT1 target transcript in PHH after treatment with EONs that were formulated in LNPs. The LNP formulations with the indicated EONs were administered to the cells in the concentration indicated on the right. The untreated sample was a single sample since no LNP was administered there. (B) shows the percentage of exon 5 skip observed in the same samples.

FIG. 11(A) shows the editing percentage of the B4GALT1 target transcript in vivo in mouse liver cells, after IV injection of LNP formulations comprising RNA editing oligonucleotides EON05, EON13, or EON134, as outlined in example 8, on day 2, 4, 7, and 30 after administration. PBS administration and an Actin B targeting oligonucleotide (RM3891) formulated in LNP served as a negative control for B4GALT1 editing and as a positive control for editing in general (on the Actin B target), which was around 40% on day 4 after administration (data not shown). (B) shows the percentage of exon 5 skip observed in the same samples.

FIG. 12 shows an additional set of 960 EONs with chemical modifications as given in FIG. 2. The respective SEQ ID NO's are given next to the RM number.

FIG. 13 shows an additional set of 22 EONs with chemical modifications as given in FIG. 2. The respective SEQ ID NO's are given next to the RM number. A “#” refers to a PNms linkage.

FIG. 14 shows the percentage editing of the B4GALT1 transcript in PHHs after treatment with the indicated EONs. B4GALT1-69=RM107689 and B4GALT1-84=RM107704 in FIG. 2.

FIG. 15(A) shows the schematic position of nine sequence sets (EONs) towards the exon 5-exon 6 boundary in the B4GALT1 transcript, with set #9 being most outside exon 5. Several EONs (details not shown) with a variety of modifications generally as outlined herein were designed for each set to test the effect on exon 5 skipping. (B) shows the results of these exon skipping experiments, with all generated EONs having separate bars, but with the nine separate sets of sequences indicated below the graph. RM105550 (=B4GALT-13 with GalNAc at the 5′ side, but without TEG linker) served as a positive control. A non-treated sample (NT) served as a negative control.

FIG. 16 shows a set of EONs with a variety of modifications related to 2′-F at different positions, with SEQ ID NO: 1101-1120 being related to the original design of SEQ ID NO:1100 (B4GALT1-13) and SEQ ID NO: 1122-1138 being related to the original design of SEQ ID NO: 1121 (B4GALT1-21). All modifications are as provided in FIGS. 2 and 6. No TEG linker is present between the L001 GalNAc moiety and the most terminal 5′ nucleotide.

FIG. 17 (A) shows the editing percentages (left y-axis) and the exon 5 skipping percentages (right y-axis) in PHHs treated with the indicated EONs. A non-treated (NT) sample served as a negative control and RM106564 (EON13) served as the control for reference. (B) shows the results of an identical experiment using a different set of EONs in which two non-treated samples served as negative controls and RM106566 (EON21) served as the reference control.

### DETAILED DESCRIPTION

It was realized that it is possible to target the B4GALT1 transcript to modulate the activity of the B4GALT1 enzyme, and thereby prevent, ameliorate, or treat CVD. This technology is generally referred to as “RNA editing”. Disclosed herein are oligonucleotides that can be used to specifically deaminate a specific target adenosine in the transcript of the (human) B4GALT1 transcript (pre-mRNA and/or mRNA) in vivo, preferably using endogenous deaminating enzymes, to produce a B4GALT1 enzyme variant with reduced galactosyltrasferase activity. A particularly preferred target adenosine is the one found in the codon for asparagine at amino acid position 352 (Asn352), wherein deamination leads to a codon that encodes serine (Ser352), but the RNA editing technology as disclosed herein is also applicable to other target adenosines within B4GALT1 that may be targeted to reduce galactosyltransferase turnover rate.

RNA editing is a natural process through which eukaryotic cells alter the sequence of their RNA molecules, often in a site-specific and precise way, thereby increasing the repertoire of genome encoded RNAs by several orders of magnitude. RNA editing enzymes have been described for eukaryotic species throughout the animal and plant kingdoms, and these processes play an important role in managing cellular homeostasis in metazoans from the simplest life forms (such as Caenorhabditis elegans) to humans. Examples of RNA editing are adenosine (A) to inosine (I) conversions and cytidine (C) to uridine (U) conversions, which occur through enzymes called Adenosine Deaminases acting on RNA (ADAR) and APOBEC/AID (cytidine deaminases that act on RNA), respectively.

ADAR is a multi-domain protein, comprising a catalytic domain, and two to three double-stranded RNA recognition domains, depending on the enzyme in question. Each recognition domain recognizes a specific double stranded RNA (dsRNA) sequence and/or conformation. The catalytic domain does also play a role in recognizing and binding a part of the dsRNA helix, although the key function of the catalytic domain is to convert an A into I in a nearby, predefined, position in the target RNA, by deamination of the nucleobase. Inosine is read as guanosine by the translational machinery of the cell, meaning that, if an edited adenosine is in a coding region of an mRNA or pre-mRNA, it can recode the protein sequence. A to I conversions may also occur in 5′ non-coding sequences of a target mRNA, creating new translational start sites upstream of the original start site, which gives rise to N-terminally extended proteins, or in the 3′ UTR or other non-coding parts of the transcript, which may affect the processing and/or stability of the RNA. In addition, A to I conversions may take place in splice elements in introns or exons in pre-mRNAs, thereby altering the pattern of splicing. As a result, exons may be included or skipped. The enzymes catalysing adenosine deamination are within an enzyme family of ADARs, which include human deaminases hADAR1 and hADAR2, as well as hADAR3. However, for hADAR3 no deaminase activity has been demonstrated.

The use of oligonucleotides to edit a target RNA applying adenosine deaminase has been described (e.g., Woolf et al. 1995. Proc Natl Acad Sci USA 92:8298-8302; Montiel-Gonzalez et al. 2013. Proc Natl Acad Sci USA 110 (45): 18285-18290; Vogel et al. 2014. Angewandte Chemie Int Ed 53:267-271). A disadvantage of the method described by Montiel-Gonzalez et al. (2013, supra) is the need for a fusion protein consisting of the boxB recognition domain of bacteriophage lambda N-protein, genetically fused to the adenosine deaminase domain of a truncated natural ADAR protein. It requires target cells to be either transduced with the fusion protein, which is a major hurdle, or that target cells are transfected with a nucleic acid construct encoding the engineered adenosine deaminase fusion protein for expression. The system described by Vogel et al. (2014, supra) suffers from similar drawbacks, in that it is not clear how to apply the system without having to genetically modify the ADAR first and subsequently transfect or transform the cells harboring the target RNA, to provide the cells with this genetically engineered protein. U.S. Pat. No. 9,650,627 describes a similar system. The oligonucleotides of Woolf et al. (1995, supra) that were 100% complementary to the target RNA sequences suffered from severe lack of specificity: nearly all adenosines in the target RNA strand that was complementary to the antisense oligonucleotide were edited.

It is known that ADAR may act on any dsRNA. Through a process sometimes referred to as ‘promiscuous editing’ the enzyme will edit multiple A's in the dsRNA. Hence, there was a need for methods and means that circumvent such promiscuous editing and only target specific adenosines in a target RNA molecule to become therapeutic applicable. Vogel et al. (2014, supra) showed that such off-target editing can be suppressed by using 2′-O-methyl(2′-OMe) modified nucleosides in the oligonucleotide at positions opposite to adenosines that should not be edited and used a non-modified nucleoside directly opposite to the specifically targeted adenosine on the target RNA. However, the specific editing effect at the target nucleotide has not been shown to take place without the use of recombinant ADAR enzymes having covalent bonds with the EON. Several publications have now shown that the recruitment of endogenous ADAR (hence without the need for an exogenous and/or recombinant source) is feasible while maintaining a specificity in which a single adenosine within a target RNA molecule can be targeted and deaminated to an inosine. WO2016/097212 discloses antisense oligonucleotides (AONs) for the targeted editing of RNA, wherein the AONs are characterized by a sequence that is complementary to a target RNA sequence (therein referred to as the ‘targeting portion’) and by the presence of a stem-loop/hairpin structure (therein referred to as the ‘recruitment portion’), which is preferably non-complementary to the target RNA. Such oligonucleotides are referred to as ‘self-looping AONs’. The recruitment portion acts in recruiting a natural ADAR enzyme present in the cell to the dsRNA formed by hybridization of the target sequence with the targeting portion. Due to the recruitment portion, there is no need for conjugated entities or presence of modified recombinant ADAR enzymes. WO2016/097212 describes the recruitment portion as being a stem-loop structure mimicking either a natural substrate (e.g., the GluB receptor) or a Z-DNA structure known to be recognized by the dsRNA binding domains, or Z-DNA binding domains, of ADAR enzymes. A stem-loop structure can be an intermolecular stem-loop structure, formed by two separate nucleic acid strands, or an intramolecular stem loop structure, formed within a single nucleic acid strand. The stem-loop structure of the recruitment portion as described is an intramolecular stem-loop structure, formed within the AON itself, and are thought to attract (endogenous) ADAR. Similar stem-loop structure-comprising systems for RNA editing have been described in WO2017/050306, WO2020/001793, WO2017/010556, WO2020/246560, and WO2022/078995.

WO2017/220751 and WO2018/041973 describe a next generation type of AONs that do not comprise such a stem-loop structure but that are (almost fully) complementary to the targeted area. In one embodiment, one or more mismatching nucleotides, wobbles, or bulges exist between the oligonucleotide and the target sequence. A sole mismatch may be at the site of the nucleoside opposite the target adenosine, but in other embodiments AONs (or RNA editing oligonucleotides, abbreviated to ‘EONs’) were described with multiple bulges and/or wobbles when attached to the target sequence area. It appeared possible to achieve in vitro, ex vivo and in vivo RNA editing with EONs lacking a stem-loop structure and with endogenous ADAR enzymes when the sequence of the EON was carefully selected such that it could attract/recruit ADAR. The ‘orphan nucleoside’, which is defined as the nucleoside in the EON that is positioned directly opposite the target adenosine in the target RNA molecule, did not carry a 2′-OMe modification. The orphan nucleoside can be a deoxyribonucleoside (DNA), wherein the remainder of the EON could still carry 2′-O-alkyl modifications at the sugar entity (such as 2′-OMe), or the nucleotides directly surrounding the orphan nucleoside contained chemical modifications (such as DNA in comparison to RNA) that further improved the RNA editing efficiency and/or increased the resistance against nucleases. Such effects could even be further improved by using sense oligonucleotides (SONs) that ‘protected’ the EONs against breakdown (described in WO2018/134301). The use of chemical modifications and particular structures in oligonucleotides that could be used in ADAR-mediated editing of specific adenosines in a target RNA have been the subject of numerous publications in the field, such as WO2019/111957, WO2019/158475, WO2020/165077, WO2020/201406, WO2020/211780, WO2021/008447, WO2021/020550, WO2021/060527, WO2021/117729, WO2021/136408, WO2021/182474, WO2021/216853, WO2021/242778, WO2021/242870, WO2021/242889, WO2022/007803, WO2022/018207, WO2022/026928, and WO2022/124345. The use of specific sugar moieties has been disclosed in for instance WO2020/154342, WO2020/154343, WO2020/154344, WO2022/103839, and WO2022/103852, whereas the use of stereo-defined linker moieties (in general for oligonucleotides that for instance can be used for exon skipping, in gapmers, in siRNA, or specifically for RNA-editing oligonucleotides, related to a wide variety of target sequences) has been described in WO2011/005761, WO2014/010250, WO2014/012081, WO2015/107425, WO2017/015575 (HTT), WO2017/062862, WO2017/160741, WO2017/192664, WO2017/192679 (DMD), WO2017/198775, WO2017/210647, WO2018/067973, WO2018/098264, WO2018/223056 (PNPLA3), WO2018/223073 (APOC3), WO2018/223081 (PNPLA3), WO2018/237194, WO2019/032607 (C9orf72), WO2019/055951, WO2019/075357 (SMA/ALS), WO2019/200185 (DM1), WO2019/217784 (DM1), WO2019/219581, WO2020/118246 (DM1), WO2020/191252, WO2020/196662, WO2020/219981 (USH2A), WO2020/219983 (RHO), WO2020/227691 (C9orf72), WO2021/071788 (C9orf72), WO2021/071858, WO2021/178237 (MAPT), WO2021/234459, WO2021/237223, and WO2022/099159. Next to these disclosures, an extensive number of publications relate to the targeting of specific RNA target molecules, or specific adenosines within such RNA target molecules, be it to repair a mutation that resulted in a premature stop codon, or other mutation causing disease. Examples of such disclosures in which adenosines are targeted within specified target RNA molecules are WO2020/157008 and WO2021/136404 (USH2A); WO2021/113270 (APP); WO2021/113390 (CMT1A); WO2021/209010 (IDUA, Hurler syndrome); WO2021/231673 and WO2021/242903 (LRRK2); WO2021/231675 (ASS1); WO2021/231679 (GJB2); WO2019/071274 and WO2021/231680 (MECP2); WO2021/231685 and WO2021/231692 (OTOF, autosomal recessive non-syndromic hearing loss); WO2021/231691 (XLRS); WO2021/231698 (argininosuccinate lyase deficiency); WO2021/130313 and WO2021/231830 (ABCA4); and WO2021/243023 (SERPINA1).

### EMBODIMENTS

According to one aspect, the disclosure provides an EON that can form a double-stranded complex with a region of an endogenous human B4GALT1 transcript molecule in a cell, wherein the region of the B4GALT1 transcript molecule, or a part thereof comprises a target adenosine, wherein the nucleotide in the EON that is opposite the target adenosine is the orphan nucleotide, and wherein the double-stranded complex can recruit an endogenous ADAR enzyme present in the cell to deaminate the target adenosine into an inosine, thereby editing the B4GALT1 transcript molecule.

According to one aspect, the disclosure provides an EON that can form a double-stranded complex with a region of an endogenous human B4GALT1 transcript molecule in a cell, wherein the region of the B4GALT1 transcript molecule, or a part thereof comprises a target adenosine, wherein the nucleotide in the EON that is opposite the target adenosine is the orphan nucleotide, and wherein the double-stranded complex can recruit an endogenous ADAR enzyme present in the cell to deaminate the target adenosine into an inosine, thereby editing the B4GALT1 transcript molecule, and wherein the EON causes a splice modulation event, preferably wherein exon 5 is skipped from the pre-mRNA, thereby removing the exon including the target adenosine (be it edited or not) from the transcript, leaving an mRNA that does not translate to a functional B4GALT1 protein. The splice modulation event may be exon 5 skipping, may be an aberrant exon skip, may be exon 4+exon 5 skipping, may be intron inclusion, and may be a (partial) skip of exon 6.

In one aspect, the B4GALT1 transcript molecule is a pre-mRNA or an mRNA molecule.

In one aspect, the cell is a human liver cell, preferably a hepatocyte.

In one aspect, the target adenosine is at a position in the B4GALT1 transcript where a guanosine results in a B4GALT1 protein variant that has a reduced enzymatic turnover rate.

In one aspect, the target adenosine is at position c. 1055A in the B4GALT1 transcript.

In one aspect, the region of the B4GALT1 transcript molecule, or a part thereof, comprises the sequence: 5′- . . . CCCAAUCCU . . . -3′, wherein A is the target adenosine.

In one aspect, the EON comprises or consists of an EON, individually selected from the group consisting of the EONs as provided in detail in SEQ ID NO:3 to 42, 59 to 1069, and 1078 to 1190, preferably from the group consisting of SEQ ID NO:23, 19, 31, 27, 35, 39, 69, 70, 71, 72, 73, 93, 94, 95, 1079, 1084, 1093, 1095, 1100, 1102, 1115, 1121, 1123, 1124, and 1139 to 1190.

In one aspect, the EON comprises or consists of the basic nucleotide sequence as provided in any one of SEQ ID NO: 1139 to 1190, wherein:
the EON may comprise a GalNAc moiety (such as the L001 disclosed herein), wherein the GalNAc moiety may be linked to the EON via a linker (such as the L103 TEG linker as disclosed herein);
the EON may comprise nucleotides that are modified in the sugar moiety as disclosed herein, preferably selected from the group consisting of LNA, 2′-MOE, 2′-OMe, 2′-F, and 2′-H (DNA);
the EON may comprise nucleotides that are modified in the nucleobase moiety as disclosed herein;
the EON may comprise nucleosides that are linked to each other with a linkage as disclosed herein, preferably selected from the group consisting of PS, PNdmi, MP, and PNms;
the EON comprises an orphan nucleotide that is preferably a deoxynucleotide that comprises a cytosine base, a uracil base, an iso-uracil base, or a cytosine analogue, more preferably a Benner's base;
the EON comprises a nucleotide at position-1 that is preferably a deoxynucleotide;
the EON comprises a nucleotide at position+1 that is preferably a deoxynucleotide or a 2′-MOE modified nucleotide;
the EON comprises a linkage at linkage position-2 that is preferably a MP linkage or a PNms linkage;
the EON comprises a linkage at linkage position-1 that is preferably a PS linkage;
the EON comprises at positions+1, 0, and −1 the sequence 5′-AXU-3′, wherein
   a) A is a nucleoside carrying an adenine base, preferably wherein A is a deoxyadenosine (Ad) or a 2′-MOE modified adenosine (Ae);
   b) X is the orphan nucleotide, which is a deoxynucleotide comprising a modification as disclosed herein, preferably comprising a Benner's base (Zd) or an iso-uracil (8d); and
   c) U is a nucleoside carrying a uracil base, preferably wherein U is a deoxyuridine (Ud, or m5Ud);
the EON may comprise a PNdmi linkage connecting the ultimate 5′ nucleoside to its neighbouring nucleoside; and/or
the EON may comprise a PNdmi linkage connecting the ultimate 3′ nucleoside to its neighbouring nucleoside.

SEQ ID NO: 1139 to 1151 are as follows (from 5′ to 3′), wherein X is the orphan nucleotide, which is preferably a deoxynucleotide carrying a Benner's base (Zd), and the sequence of positions +1, 0, and −1 (sometimes referred to as the “Central Triplet”) is underlined:

1139
CCUCUGAGGAXUGGGUUCAUUU
1140
CCUCUGAGGAXUGGGUUCAUUUUU
1141
AACCUCUGAGGAXUGGGUUCAUUU
1142
AACCUCUGAGGAXUGGGUUCAUUUU
1143
ACCUCUGAGGAXUGGGUUCAUUUUU
1144
AACCUCUGAGGAXUGGGUUCAUUUUU
1145
CACCUCUGAGGAXUGGGUUCAUUUUU
1146
GCACCUCUGAGGAXUGGGUUCAUUUUU
1147
CAAACCUCUGAGGAXUGGGUUCAUUUUU
1148
CGGUCAAACCUCUGAGGAXUGGGUUCAU
1149
GUCAAACCUCUGAGGAXUGGGUUCAUUUUU
1150
CGGUCAAACCUCUGAGGAXUGGGUUCAUUU
1151
CGGUCAAACCUCUGAGGAXUGGGUUCAUUUUU

SEQ ID NO:1152 to 1164 are as follows (from 5′ to 3′), wherein X is the orphan nucleotide, which is preferably a deoxycytidine, and the sequence of positions +1, 0, and −1 (sometimes referred to as the “Central Triplet”) is underlined:

1152
CCUCUGAGGAXUGGGUUCAUUU
1153
CCUCUGAGGAXUGGGUUCAUUUUU
1154
AACCUCUGAGGAXUGGGUUCAUUU
1155
AACCUCUGAGGAXUGGGUUCAUUUU
1156
ACCUCUGAGGAXUGGGUUCAUUUUU
1157
AACCUCUGAGGAXUGGGUUCAUUUUU
1158
CACCUCUGAGGAXUGGGUUCAUUUUU
1159
GCACCUCUGAGGAXUGGGUUCAUUUUU
1160
CAAACCUCUGAGGAXUGGGUUCAUUUUU
1161
CGGUCAAACCUCUGAGGAXUGGGUUCAU
1162
GUCAAACCUCUGAGGAXUGGGUUCAUUUUU
1163
CGGUCAAACCUCUGAGGAXUGGGUUCAUUU
1164
CGGUCAAACCUCUGAGGAXUGGGUUCAUUUUU

SEQ ID NO:1165 to 1177 are as follows (from 5′ to 3′), wherein X is the orphan nucleotide, which is preferably a deoxyuridine, and the sequence of positions +1, 0, and −1 (sometimes referred to as the “Central Triplet”) is underlined:

1165
CCUCUGAGGAXUGGGUUCAUUU
1166
CCUCUGAGGAXUGGGUUCAUUUUU
1167
AACCUCUGAGGAXUGGGUUCAUUU
1168
AACCUCUGAGGAXUGGGUUCAUUUU
1169
ACCUCUGAGGAXUGGGUUCAUUUUU
1170
AACCUCUGAGGAXUGGGUUCAUUUUU
1171
CACCUCUGAGGAXUGGGUUCAUUUUU
1172
GCACCUCUGAGGAXUGGGUUCAUUUUU
1173
CAAACCUCUGAGGAXUGGGUUCAUUUUU
1174
CGGUCAAACCUCUGAGGAXUGGGUUCAU
1175
GUCAAACCUCUGAGGAXUGGGUUCAUUUUU
1176
CGGUCAAACCUCUGAGGAXUGGGUUCAUUU
1177
CGGUCAAACCUCUGAGGAXUGGGUUCAUUUUU

SEQ ID NO: 1178 to 1190 are as follows (from 5′ to 3′), wherein X is the orphan nucleotide, which is preferably a deoxynucleotide carrying an iso-uracil base, and the sequence of positions +1, 0, and −1 (sometimes referred to as the “Central Triplet”) is underlined:

1178
CCUCUGAGGAXUGGGUUCAUUU
1179
CCUCUGAGGAXUGGGUUCAUUUUU
1180
AACCUCUGAGGAXUGGGUUCAUUU
1181
AACCUCUGAGGAXUGGGUUCAUUUU
1182
ACCUCUGAGGAXUGGGUUCAUUUUU
1183
AACCUCUGAGGAXUGGGUUCAUUUUU
1184
CACCUCUGAGGAXUGGGUUCAUUUUU
1185
GCACCUCUGAGGAXUGGGUUCAUUUUU
1186
CAAACCUCUGAGGAXUGGGUUCAUUUUU
1187
CGGUCAAACCUCUGAGGAXUGGGUUCAU
1188
GUCAAACCUCUGAGGAXUGGGUUCAUUUUU
1189
CGGUCAAACCUCUGAGGAXUGGGUUCAUUU
1190
CGGUCAAACCUCUGAGGAXUGGGUUCAUUUUU

In one aspect, at least one nucleotide of the EON as disclosed herein comprises one or more non-naturally occurring chemical modifications, or one or more additional non-naturally occurring chemical modifications, in the ribose, linkage, or base moiety, with the proviso that the orphan nucleotide is not a cytidine comprising a 2′-OMe ribose substitution.

In one aspect, the one or more additional modifications in the linkage moiety is each independently selected from a PS, phosphonoacetate, phosphorodithioate, MP, sulfonylphosphoramidate, or PNdmi internucleotide linkage.

In one aspect, the EON as disclosed herein comprises one or more nucleotides comprising a mono- or di-substitution at the 2′, 3′ and/or 5′ position of the ribose, each independently selected from the group consisting of: —OH; —F; substituted or unsubstituted, linear or branched lower (C₁-C₁₀) alkyl, alkenyl, alkynyl, alkaryl, allyl, or aralkyl, that may be interrupted by one or more heteroatoms; —O-, S-, or N-alkyl; —O-, S-, or N-alkenyl; —O-, S-, or N-alkynyl; —O—, S-, or N-allyl; —O-alkyl-O-alkyl; -methoxy; -aminopropoxy; -methoxyethoxy; -dimethylamino oxyethoxy; and -dimethylaminoethoxyethoxy.

According to one aspect, disclosed herein is a vector, preferably a viral vector, more preferably an AAV vector, comprising a nucleic acid molecule encoding an EON as disclosed herein.

According to one aspect, disclosed herein is a nanoparticle delivery vehicle formulation comprising an EON as disclosed herein. Preferably, the nanoparticle delivery vehicle formulation as disclosed herein is a LNP formulation.

According to one aspect, disclosed herein is a method of editing a B4GALT1 polynucleotide, the method comprising contacting the B4GALT1 polynucleotide with an EON as disclosed herein to cause an adenosine deaminase acting on RNA (ADAR)-mediated adenosine to inosine alteration of an adenosine associated with CVD, thereby editing the B4GALT1 polynucleotide. The polynucleotide is preferably a pre-mRNA or an mRNA molecule.

According to one aspect, disclosed herein is a method of treating CVD in a patient in need thereof, the method comprising contacting a B4GALT1 polynucleotide in a cell of the subject with an EON, a vector, a nanoparticle delivery vehicle formulation, or a pharmaceutical composition as disclosed herein to cause an ADAR-mediated adenosine to inosine alteration of an adenosine associated with CVD, thereby treating the patient.

According to one aspect, disclosed herein is a method of treating CVD, the method comprising administering to a patient in need thereof a therapeutically effective amount of an EON, a vector, a nanoparticle delivery vehicle formulation, or a pharmaceutical composition as disclosed herein, thereby treating the CVD.

According to one aspect, herein is provided a method for the deamination of a target adenosine in a human B4GALT1 pre-mRNA or mRNA molecule in a cell, the method comprising the steps of: (i) providing the cell with an EON as disclosed herein; (ii) allowing uptake by the cell of the EON; (iii) allowing annealing of the EON to the B4GALT1 pre-mRNA or mRNA molecule; (iv) allowing an endogenous ADAR enzyme to deaminate the target adenosine in the target RNA molecule to an inosine; and optionally (v) identifying the presence of the inosine in the target RNA molecule. In one aspect, the target adenosine is the c. 1055A position in the B4GALT1 pre-mRNA or mRNA molecule. In one aspect, step (v) comprises: a) determining the sequence of the B4GALT1 pre-mRNA or mRNA molecule; b) assessing the presence of a 352Ser B4GALT1 protein variant; or c) using a functional read-out, preferably assessing a reduction rate of UDP-Gal, or assessing glycosylation levels of transferrin in serum.

The present disclosure provides EONs that can produce RNA editing of a target adenosine in the human B4GALT1 transcript (pre-mRNA and/or mRNA) which produces a B4GALT1 enzyme variant that has a reduced turnover rate. In one aspect, the EON results in at least 1%, 2%, 3%, 4%, 5%, 6%, 7%, 8%, 9%, 10%, 15%, 20%, 25%, 30%, 35%, 40%, 45%, 50%, 60%, 70%, 80%, 90% or 100% of the B4GALT1 transcript molecules encoding variant B4GALT1. The target adenosine may be at any position whereby editing results in a reduced turnover rate. Any reduction in turnover rate with respect to the wild-type enzyme can be beneficial. In one embodiment, the reduction in turnover rate of the B4GALT1 variant is at least a 1%, 2%, 3%, 4%, 5%, 6%, 7%, 8%, 9%, 10%, 15%, 20%, 25%, 30%, 35%, 40%, 45%, 50%, 60%, 70%, 80%, 90% or 100% reduction in turnover rate of the variant with respect to the wild-type enzyme. In one embodiment, the reduction in turnover rate can be measured by determining the rate at which the B4GALT1 enzyme can transfer galactose from UDP-Gal to GlcNAc as an acceptor, as described in Montasser et al. (2021, supra). Editing of the target adenosine may reduce turnover rate of the B4GALT1 enzyme variant through different mechanisms, including, for example, mutation of an amino acid residue, generation of an early stop codon, or variation of pre-mRNA splice sites.

It is additionally noted that the reduction in turnover rate in the system as a whole (for example, in a cellular environment) can depend upon multiple factors, including the reduction in turnover rate of any given enzyme variant, and the ratio of enzyme variant to wild-type enzyme produced in that system, and these can be balanced to produce the desired effect. For instance, Montasser et al. (2021, supra) describe the use of a different editing technique, a gene editing technique that used CRISPR-Cas9 knockdown of the B4GALT1 gene (i.e., to encode fully inactivated B4GALT1). In this model, gene editing was not exhaustive, resulting in 20.7% of the transcripts being active wild-type B4GALT1. This also resulted in an appreciable reduction (50%) in LDL-C. One advantage of the RNA editing approach of the present disclosure is that RNA editing is transient, allowing for much simpler tailoring of the level of transcripts being edited. This can be particularly useful if the level of reduction should be re-evaluated and adjusted on an ongoing basis, depending, for example, on disease progression/regression and or changes in substrate levels in the system.

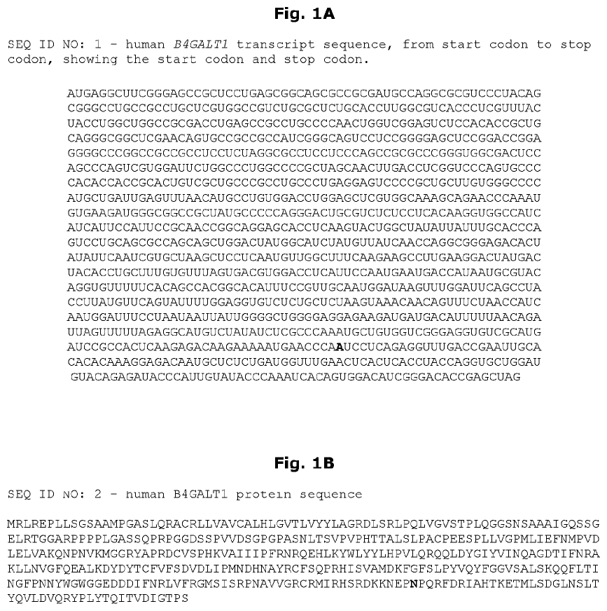

In one aspect, the reduction in turnover rate in the system as a whole is at least a 1%, 2%, 3%, 4%, 5%, 6%, 7%, 8%, 9%, 10%, 15%, 20%, 25%, 30%, 35%, 40%, 45% or 50% reduction in turnover rate of the system including the EON with respect to the system without the EON. The ratio of enzyme variant to wild-type enzyme produced in a system can be controlled by choice of reaction conditions (such as concentrations of reactants or choice of temperatures), though in a therapeutic setting this will often be dependent on the dose of EON provided. The skilled person can work with and optimise these factors accordingly to produce optimal reductions in turnover rate in the system as a whole. In one example, the B4GALT1 enzyme variant may be non-functional, but the dose of EON may be controlled so that the system as a whole may still contain a certain level of functional wild-type B4GALT1. This is an important advantage of RNA editing versus DNA editing because editing of the DNA to encode a non-functional B4GALT1 would cause all B4GALT1 enzyme molecules to be non-functional. In one aspect, the wild-type enzyme has the sequence shown in FIG. 1B.

In one aspect, the EON causes the deamination of the adenosine present at position 1055 of the human mRNA, thereby generating an inosine. In other words, the AAU codon encoding asparagine at amino acid position 352 is converted to an AIU codon, which is read as AGU that encodes serine. In another aspect, an EON as disclosed herein causes the deamination of another adenosine present in the B4GALT1 transcript, which may be any adenosine that, when deaminated into an inosine, results in a B4GALT1 enzyme with a decreased turnover rate. Other mutations may additionally be made in the B4GALT1 gene (and transcript), that may be brought about through RNA editing, to reduce the normal B4GALT1 function. It is to be understood that ‘causing’, ‘triggering’, or ‘producing’ deamination does not mean that the EON itself is editing the adenosine (or has enzymatic activity). The double-stranded complex of the EON with the target RNA molecule is bound to the ADAR enzyme, which acts as the deaminating entity. The ADAR is the enzyme that deaminates, but the EON is responsible for triggering this, at the wanted site.

In one aspect, the EON as disclosed herein is a single-stranded oligonucleotide comprising an orphan nucleotide that is positioned opposite the target adenosine, wherein the orphan nucleotide is chemically modified as disclosed herein, and wherein the remainder of the oligonucleotide is chemically modified to prevent it from nuclease breakdown also as disclosed herein. In one aspect, the disclosure relates to any kind of oligonucleotide or heteroduplex oligonucleotide complex, that may or may not be bound to hairpin structures (internally or at the terminal end(s)), that may be bound to ADAR or catalytic domains thereof, or wherein the oligonucleotide is expressed through a vector, such as an AAV, or wherein the oligonucleotide is in a circular format. It is to be understood that any kind of oligonucleotide-based RNA editing is encompassed if it relates to the deamination of a nucleotide in the B4GALT1 transcript, preferably to generate the enzyme variant p.Asn352Ser, and causes the reduction in B4GALT1 enzyme function. The protein mutation referred to as p.Asn352Ser may also be referred to as N352S, whereas the adenosine change to guanosine at position 1055 in the B4GALT1 transcript may also be referred to as c. 1055A>G. In one aspect, the EON as disclosed herein is a ‘naked’ oligonucleotide, comprising a variety of chemical modifications in the ribose sugar, the base, and/or the internucleoside linkage of one or more of the nucleotides within the sequence, that can hybridize to the B4GALT1 transcript or a part thereof that includes the target adenosine, and can recruit endogenous ADAR for the deamination of the target adenosine.

### Definitions

The term ‘nucleoside’ refers to the nucleobase linked to the (deoxy) ribosyl sugar, without phosphate groups. A ‘nucleotide’ is composed of a nucleoside and one or more phosphate groups. The term ‘nucleotide’ thus refers to the respective nucleobase-(deoxy) ribosyl-phospholinker, as well as any chemical modifications of the ribose moiety or the phospho group. Thus, the term would include a nucleotide including a locked ribosyl moiety (comprising a 2′-4′ bridge, comprising a methylene group or any other group), an unlocked nucleic acid (UNA), a threose nucleic acid (TNA), a nucleotide including a linker comprising a phosphodiester, phosphonoacetate, phosphotriester, PS, phosphoro(di)thioate, MP, methyl thiophosphonate, phosphoramidate linkages, and the like. Sometimes the terms adenosine and adenine, guanosine and guanine, cytidine and cytosine, uracil and uridine, thymine and thymidine/uridine, inosine, and hypoxanthine, are used interchangeably to refer to the corresponding nucleobase on the one hand, and the nucleoside or nucleotide on the other. Thymine (T) is also known as 5-methyluracil (m⁵U) and is a uracil (U) derivative; thymine, 5-methyluracil and uracil can be interchanged throughout the disclosure. Likewise, thymidine is also known as 5-methyl-uridine and is a uridine derivative; thymidine, 5-methyl-uridine and uridine can be interchanged throughout the disclosure. Sometimes the terms nucleobase, nucleoside and nucleotide are used interchangeably, unless the context clearly requires differently, for instance when a nucleoside is linked to a neighbouring nucleoside and the linkage between these nucleosides is modified. As stated herein, a nucleotide is a nucleoside plus one or more phosphate groups. The terms ‘ribonucleoside’ and ‘deoxyribonucleoside’, or ‘ribose’ and ‘deoxyribose’ are as used in the art.

Whenever reference is made to an oligonucleotide, oligo, ON, ASO, oligonucleotide composition, antisense oligonucleotide, AON, (RNA) editing oligonucleotide, EON, and RNA (antisense) oligonucleotide, both oligoribonucleotides and deoxyoligoribonucleotides are meant unless the context dictates otherwise. Potentially the oligonucleotide may completely lack RNA or DNA nucleotides (as they appear in nature) and may consist completely of modified nucleotides. Whenever reference is made to an ‘oligoribonucleotide’ it may comprise the bases A, G, C, U, or I. Whenever reference is made to a ‘deoxyoligoribonucleotide’ it may comprise the bases A, G, C, T, or I. However, an oligonucleotide as disclosed herein may comprise a mix of ribonucleosides and deoxyribonucleosides. When a deoxyribonucleotide is used, hence without a modification at the 2′ position of the sugar, the nucleotide is often abbreviated to Ad. Cd, Gd, Id, Ud, or T in which the ‘d’ represents the deoxy nature of the nucleoside, while a ribonucleoside that is either normal RNA or modified at the 2′ position is often abbreviated without the ‘d’, and often abbreviated with their respective modifications and as explained herein.

Whenever reference is made to nucleotides in the oligonucleotide, such as cytosine, 5-methylcytosine, 5-hydroxymethylcytosine, 5-formylcytosine, 5-acetylcytosine, 5-hydroxycytosine, and β-D-glucosyl-5-hydroxymethylcytosine are included. Whenever reference is made to adenine, N6-methyladenine, 8-oxo-adenine, 2,6-diaminopurine and 7-methyladenine are included. Whenever reference is made to uracil, dihydrouracil, iso-uracil, N3-glycosylated uracil, pseudouracil, 5-methyluracil, N1-methylpseudouracil, 4-thiouracil and 5-hydroxymethyluracil are included. Whenever reference is made to guanine, 1-methylguanine, 7-methylguanosine, N2,N2-dimethylguanosine, N2,N2,7-trimethylguanosine and N2,7-dimethylguanosine are included. Whenever reference is made to nucleosides or nucleotides, ribofuranose derivatives, such as 2′-deoxy, 2′-hydroxy, and 2′-O-substituted variants, such as 2′-OMe, are included, as well as other modifications, including 2′-4′ bridged variants. Whenever reference is made to oligonucleotides, linkages between two mononucleotides may be phosphodiester linkages as well as modifications thereof, including, phosphonoacetate, phosphotriester, PS, phosphoro(di)thioate, MP, phosphoramidate linkers, phosphoryl guanidine, thiophosphoryl guanidine, sulfono phosphoramidate and the like.

The term ‘comprising’ encompasses ‘including’ as well as ‘consisting of’, e.g., a composition ‘comprising X’ may consist exclusively of X or may include something additional, e.g., X+Y. The term ‘about’ in relation to a numerical value x is optional and means, e.g., x±10%.

The word ‘substantially’ does not exclude ‘completely’, e.g., a composition which is ‘substantially free from Y’ may be completely free from Y. Where relevant, the word ‘substantially’ may be omitted from the definition.

The term ‘complementary’ as used herein refers to the fact that the EON hybridizes under physiological conditions to a second nucleic acid strand (for instance when the oligonucleotide as a first nucleic acid strand (=guide oligonucleotide) forms a heteroduplex RNA editing oligonucleotide complex, or HEON, with another complementary nucleic acid strand), or when it forms a double stranded complex with the target RNA sequence. The term does not necessarily mean that each nucleotide in a nucleic acid strand has a perfect pairing with its opposite nucleotide in the opposite sequence. In other words, while an EON may be complementary to a target sequence, there may be mismatches, wobbles and/or bulges between the oligonucleotide and the target sequence, while under physiological conditions that EON still hybridizes to the target sequence such that the cellular RNA editing enzymes can edit the target adenosine. The term ‘substantially complementary’ therefore also means that despite the presence of the mismatches, wobbles, and/or bulges, the EON has enough matching nucleotides between the EON and target sequence that under physiological conditions the EON hybridizes to the target RNA. As shown herein, an EON may be complementary, but may also comprise one or more mismatches, wobbles and/or bulges with the target sequence, if under physiological conditions the EON is able to hybridize to its target.

The term ‘downstream’ in relation to a nucleic acid sequence means further along the sequence in the 3′ direction; the term ‘upstream’ means the converse. Thus, in any sequence encoding a polypeptide, the start codon is upstream of the stop codon in the sense strand but is downstream of the stop codon in the antisense strand.

References to ‘hybridisation’ typically refer to specific hybridisation and exclude non-specific hybridisation. Specific hybridisation can occur under experimental conditions chosen, using techniques well known in the art, to ensure that most stable interactions between probe and target are where the probe and target have at least 70%, preferably at least 80%, more preferably at least 90% sequence identity.

The term ‘mismatch’ is used herein to refer to opposing nucleotides in a double stranded RNA complex which do not form perfect base pairs according to the Watson-Crick base pairing rules. In the historical sense, mismatched nucleotides are G-A, C-A, U-C, A-A, G-G, C-C, U-U pairs. In some aspects, an EON as disclosed herein comprises fewer than four mismatches with the target sequence, for example 0, 1 or 2 mismatches. ‘Wobble’ base pairs are G-U, I-U, I-A, and I-C base pairs. Although a G: G pairing would be considered a mismatch, that does not necessarily mean that the interaction is unstable, which means that the term ‘mismatch’ may be somewhat outdated based on the prior art and the current disclosure where a Hoogsteen base-pairing may be seen as a mismatch based on the origin of the nucleotide but still be relatively stable. An isolated G: G pairing in duplex RNA can for instance be quite stable, but still be defined as a mismatch.

The term ‘splice mutation’ relates to a mutation in a gene that encodes for a pre-mRNA, wherein the splicing machinery is dysfunctional in the sense that splicing of introns from exons is disturbed and due to the aberrant splicing, the subsequent translation is out of frame resulting in premature termination of the encoded protein. Often such shortened proteins are degraded rapidly and do not have any functional activity.

An EON (and the complementary nucleic acid strand when two oligonucleotides form a HEON) as disclosed herein may be chemically modified almost in its entirety, for example by providing nucleotides with a ribose sugar moiety carrying a 2′-OMe substitution, a 2′-F substitution, or a 2′-O-methoxyethyl(2′-MOE) substitution. The orphan nucleotide in the EON is preferably a cytidine or analog thereof (such as a nucleotide carrying a Benner's base), or a uridine or analog thereof (such as iso-uridine), and/or in one aspect comprises a diF modification at the 2′ position of the sugar, in another aspect comprises a deoxyribose (2′-H, DNA), and in yet a further aspect, at least one and in another embodiment both the two neighbouring nucleotides flanking the orphan nucleotide do not comprise a 2′-OMe modification. Complete modification wherein all nucleotides of the oligonucleotide hold a 2′-OMe modification, with natural bases, results in a non-functional oligonucleotide as far as RNA editing goes (known in the art), presumably because it hinders the ADAR activity at the targeted position. In general, an adenosine in a target RNA can be protected from editing by providing an opposing nucleotide with a 2′-OMe group (at least when there are no other chemical substitutions or modifications within the nucleotide), or by providing a guanine or adenine as opposing base, as these two nucleobases are also able to reduce editing of the opposing adenosine.

Various chemistries and modifications are known in the field of oligonucleotides that can be readily used in accordance with the disclosure. The regular internucleosidic linkages between the nucleotides may be altered by mono- or di-thioation of the phosphodiester bonds to yield PS esters or phosphorodithioate esters, respectively. Other modifications of the internucleosidic linkages are possible, including amidation and peptide linkers.

In one aspect, the EON as disclosed herein comprises 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides.

It is known in the art that RNA editing entities (such as human ADAR enzymes) edit dsRNA structures with varying specificity, depending on several factors. One important factor is the degree of complementarity of the two strands making up the dsRNA sequence. Perfect complementarity of the two strands usually causes the catalytic domain of human ADAR to deaminate adenosines in a non-discriminative manner, reacting with any adenosine it encounters. The specificity of hADAR1 and 2 can be increased by introducing chemical modifications and/or ensuring several mismatches in the dsRNA, which presumably helps to position the dsRNA binding domains in a way that has not been clearly defined yet. Additionally, the deamination reaction itself can be enhanced by providing an oligonucleotide that comprises a mismatch opposite the adenosine to be edited. Following the instructions in the present disclosure, those of skill in the art will be capable of designing the complementary portion of the oligonucleotide according to their needs.

The RNA editing protein present in the cell that is of most interest to be used with an EON of the present disclosure is human ADAR1 and/or ADAR2. It will be understood by a person having ordinary skill in the art that the extent to which the editing entities inside the cell are redirected to other target sites may be regulated by varying the affinity of the EON for the recognition domain of the editing enzyme. The exact modification may be determined through some trial and error and/or through computational methods based on structural interactions between the EON and the recognition domain of the editing enzyme. In addition, or alternatively, the degree of recruiting and redirecting the editing enzyme resident in the cell may be regulated by the dosing and the dosing regimen of the EON. This is something to be determined by the experimenter (in vitro) or the clinician, usually in phase I and/or II clinical trials.

The disclosure provides the modification of target RNA sequences in eukaryotic, preferably metazoan, more preferably mammalian, most preferably human cells. The disclosure is particularly suitable for modifying RNA sequences in cells and tissues in which B4GALT1 is expressed and wherein that enzyme acts. B4GALT1 is an enzyme that plays a crucial role in the synthesis of complex carbohydrates called glycoconjugates. While it has been shown that a reduction in the enzymatic activity of B4GALT1 can have a protective effect against CVD, the exact mechanisms through which this occurs are not fully understood. B4GALT1 is involved in the construction of oligosaccharides with specific sequences and structures, particularly those that are part of peptidoglycans. These oligosaccharides are essential for the proper function and stability of proteins. A reduction in B4GALT1 activity can lead to a decrease in the level of complete oligosaccharides and downstream biological activity. One important class of oligosaccharides that B4GALT1 is involved in constructing is those terminated by sialyl groups, where sialylation relies on a preceding galactosylation step. Montasser et al. (2021, supra) found that a reduction in B4GALT1 activity is associated with decreased galactosylation and sialylation of apolipoprotein B100, fibrinogen, immunoglobulin G, and transferrin.

The target cell can be located in vitro, ex vivo or in vivo. One advantage of the material of the disclosure is that it can be used with cells in situ in a living organism, but it can also be used with cells in culture. In some aspects cells are treated ex vivo and are then introduced into a living organism (e.g., re-introduced into an organism from whom they were originally derived). The disclosure can also be used to edit target RNA sequences in cells from a transplant or within a so-called organoid, e.g., a liver tissue organoid or ‘spheroid’. Organoids can be thought of as three-dimensional in vitro-derived tissues but are driven using specific conditions to generate individual, isolated tissues. In a therapeutic setting they are useful because they can be derived in vitro from a patient's cells, and the organoids can then be re-introduced to the patient as autologous material which is less likely to be rejected than a normal transplant.

Without wishing to be bound by theory, the RNA editing through ADAR is thought to take place on primary transcripts in the nucleus, during transcription or splicing, or in the cytoplasm, where e.g., mature mRNA, miRNA or ncRNA can be edited.

It should be clear, that targeted editing according to the disclosure can be applied to any adenosine within the B4GALT1 transcript if the deamination of the adenosine results in a reduction of B4GALT1 function. As outlined herein, it is however preferred to target the adenosine at position 1055 in the wild-type B4GALT1 transcript product to yield a change from an AAU codon (encoding asparagine) to AIU (or AGU, encoding serine). Generally spoken, RNA editing may be used to create RNA sequences with different properties. Such properties may be coding properties (creating proteins with different sequences or length, leading to altered protein properties or functions), or binding properties (causing inhibition or over-expression of the RNA itself or a target or binding partner; entire expression pathways may be altered by recoding miRNAs or their cognate sequences on target RNAs). Protein function or localization may be changed at will, by functional domains or recognition motifs, including but not limited to signal sequences, targeting or localization signals, recognition sites for proteolytic cleavage or co- or post-translational modification, catalytic sites of enzymes, binding sites for binding partners, signals for degradation or activation and so on. As disclosed herein, an EON as disclosed herein may also cause splice effects, such as exon skipping (e.g., skipping of exon 5), which is not necessarily a bad thing, because the resulting mRNA may encode an inactive B4GALT1 protein, in a transient manner (because the original DNA encoding the protein remains untouched). These and other forms of RNA and protein “engineering”, whether to prevent, delay or treat disease or for any other purpose, in medicine or biotechnology, as diagnostic, prophylactic, therapeutic, research tool or otherwise, are encompassed by the present disclosure. Hence, any RNA editing of a target adenosine in the B4GALT1 transcript and that results in reduction of B4GALT1 turnover rate is encompassed by what is disclosed herein.

The present disclosure relates to a whole new field of treating CVD using genetic editing techniques. The genetic editing technique is not particularly limited. Suitable techniques include known gene therapy techniques, which include DNA editing techniques such as CRISPR/Cas, ZFNs, TALENs, and meganucleases, and preferably RNA editing techniques such as ADAR-mediated editing techniques, as further outlined in detail herein.

The amount of EON to be administered, the dosage and the dosing regimen can vary from cell type to cell type, the disease to be treated, the target population, the mode of administration (e.g., systemic versus local), the severity of disease and the acceptable level of side activity, but these can and should be assessed by trial and error during in vitro research, in pre-clinical and clinical trials. The trials are particularly straightforward when the modified sequence leads to an easily detected phenotypic change, or a change in (the level of, or activity of) a specified biomarker. It is possible that higher doses of EONs could compete for binding to an ADAR within a cell, thereby depleting the amount of the entity, which is free to take part in RNA editing, but routine dosing trials will reveal any such effects for a given EON and a given target.

One suitable trial technique involves delivering the EON to cell lines, or a test organism and then taking biopsy samples at various time points thereafter. The sequence of the target RNA can be assessed in the biopsy sample and the proportion of cells having the modification can easily be followed. After this trial has been performed once then the knowledge can be retained, and future delivery can be performed without needing to take biopsy samples. A method as disclosed herein can thus include a step of identifying the presence of the desired change in the cell's target RNA sequence, thereby verifying that the target RNA sequence has been modified. This step will typically involve sequencing of the relevant part of the target RNA, or a cDNA copy thereof (or a cDNA copy of a splicing product thereof, in case the target RNA is a pre-mRNA), as discussed above, and the sequence change can thus be easily verified. Alternatively, the change may be assessed on the function of the protein, for instance by measuring the reduction rate of UDP-Gal, or assessing glycosylation levels of transferrin in serum, before and after treatment, or any other potential marker, which measurements are preferably performed in vitro on samples obtained from the treated subject.

After RNA editing has occurred in a cell, the modified RNA can become diluted over time, for example due to cell division, limited half-life of the edited RNAs, etc. Thus, in practical therapeutic terms a method as disclosed herein may involve repeated delivery of an EON until enough target RNAs have been modified to provide a tangible benefit to the patient and/or to maintain the benefits over time.

EONs as disclosed herein are particularly suitable for therapeutic use, and so the disclosure also relates to a pharmaceutical composition comprising an EON as disclosed herein, or a vector or plasmid encoding the EON as disclosed herein, and a pharmaceutically acceptable carrier. In some aspects, the pharmaceutically acceptable carrier can simply be a saline solution. This can usefully be isotonic or hypotonic, particularly for pulmonary delivery. The disclosure also provides a delivery device (e.g., syringe, inhaler, nebuliser) which includes a pharmaceutical composition as disclosed herein.

The disclosure also provides an EON as disclosed herein for use in the treatment of CVD. This treatment can be achieved through making a change in a target B4GALT1 RNA sequence in a mammalian, such as a human liver cell, preferably a hepatocyte, because expression of B4GALT1 is high in liver, although it is found to be ubiquitously expressed throughout the human body. Similarly, the disclosure provides the use of an EON as disclosed herein in the manufacture of a medicament for making a change in a target B4GALT1 RNA sequence in a mammalian, preferably a human liver cell, more preferably a hepatocyte, as described herein, and thereby treating, preventing, or ameliorating CVD.

The disclosure also provides a method for the deamination of at least one specific target adenosine present in a target B4GALT1 RNA sequence in a cell, the method comprising the steps of: providing the cell with an EON as disclosed herein (either naked, or through vector delivery); allowing uptake by the cell of the EON (or the vector); allowing annealing of the EON to the target RNA molecule; allowing an endogenous mammalian ADAR enzyme to deaminate the target adenosine in the target RNA molecule (preferably the adenosine at position 1055 in the B4GALT1 transcript product) to an inosine; and optionally identifying the presence of the inosine in the RNA sequence.

The term CVD, includes conditions such as CAD, sometimes known as coronary heart disease, strokes and transient ischaemic attack (TIA; or mini stroke), peripheral arterial disease, and/or aortic disease. The EONs as disclosed herein may be used in the treatment, prevention, or amelioration of any or all these conditions. In a preferred embodiment, the CVD for treatment according to the disclosure is CAD.

The methods as disclosed herein can be applied to subjects where the target is an adenosine. For instance, where the target adenosine is at position 1055 of the B4GALT1 transcript, treatment will generally not be performed on a patient identified as already having, or having a likelihood of having, the c. 1055A>G variant.

The present disclosure also provides a method for the deamination of at least one specific target adenosine present in a target B4GALT1 RNA sequence in a cell, the method comprising the steps of: providing the cell with a vector or plasmid encoding the EON as disclosed herein; allowing uptake by the cell of the vector or plasmid; allowing annealing of the EON to the target RNA molecule; allowing an endogenous mammalian ADAR enzyme to deaminate the target adenosine in the target RNA molecule (preferably the adenosine at position 1055 in the B4GALT1 transcript product) to an inosine; and optionally identifying the presence of the inosine in the RNA sequence.

In a preferred aspect, depending on the ultimate effect of A to I conversion, the identification step comprises the following steps: sequencing the target RNA; sequencing cDNA derived from the target RNA; assessing the presence or absence of an A to G conversion in target RNA derived cDNA; assessing the presence or absence of a functional protein; assessing whether splicing of the pre-mRNA was altered by the deamination; or using a functional read-out, where the target RNA after the deamination should encode an enzyme with a reduced enzymatic turnover rate. Examples include assessing the reduction rate of UDP-Gal and/or assessing biomarkers in serum and/or plasma. For instance, a reduction in B4GALT1 enzymatic turnover can be detected through a reduction in fibrinogen in plasma, a decrease of LDL-C in serum, or through a decrease in serum of tetrasialylated transferrin levels with a corresponding increase in lower sialylation levels (such as an increase in trisialylated transferrin levels).

A very suitable manner to identify the presence of an inosine after deamination of the target adenosine is of course dPCR or even sequencing, using methods that are well-known to the person skilled in the art, and as outlined herein. However, the person skilled in the art of liver disease may apply tests to monitor certain biomarkers related to LDL-C and or fibrinogen levels, as discussed above.

Suitable functional assays to test for a reduction in B4GALT1 turnover are described in detail in Montasser et al. (2021, supra). In brief, one in vivo test is the carbohydrate-deficient transferrin (CDT) test. The CDT test is used clinically to diagnose patients with congenital glycosylation disorders. The CDT test assesses the level of sialylation of the protein transferrin. Under normal conditions, transferrin is mostly tetrasialylated. As B4GALT1 activity drops, the level of tetrasialylated transferrin drops and, correspondingly, the levels of lesser sialylated transferrin (such as trisialylated transferrin) increase. This test can be used to assay the effectiveness of EONs in an in vivo setting, analysing the combined effect of any reduction in B4GALT1 turnover rate along with the effect of the percentage of B4GALT1 that is the variant versus the wild type. A further test is to assess the reduction rate of UDP-Gal in a biochemical assay. Any acceptor can be used for this assay, though a convenient acceptor is the monosaccharide N-acetylglucosamine (NGlcNAc). Standard assay conditions are set out in Montasser et al. (2021, supra). This assay can find utility in examining kinetic parameters of a pure B4GALT1 mutant, or of cellular isolates after EON-mediated ADAR editing. A further test is to knock-in a B4GALT1 enzyme or variant, for example into a mouse model, and assess serum and/or plasma concentrations of biomarkers for B4GALT1 activity, such as LDL-C and/or fibrinogen.

The EON as disclosed herein is suitably administrated in aqueous solution, e.g. saline, or in suspension, optionally comprising additives, excipients and other ingredients, compatible with pharmaceutical use, at concentrations ranging from 1 ng/ml to 1 g/ml, preferably from 10 ng/ml to 500 mg/ml, more preferably from 100 ng/ml to 100 mg/ml. Dosage may suitably range from between about 1 μg/kg to about 100 mg/kg, preferably from about 10 μg/kg to about 10 mg/kg, more preferably from about 100 μg/kg to about 1 mg/kg. Administration may be by inhalation (e.g., through nebulization), intranasally, orally, by injection or infusion, intravenously, subcutaneously, intradermally, intramuscularly, intra-tracheally, intra-peritoneally, intrarectally, intrathecally, intra-cisterna magna, parenterally, and the like. Administration may be in solid form, in the form of a powder, a pill, a gel, a solution, a slow-release formulation, or in any other form compatible with pharmaceutical use in humans.

In one aspect, a method as disclosed herein comprises the steps of administering to the subject an EON or pharmaceutical composition as disclosed herein, allowing the formation of a double stranded nucleic acid complex of the EON with its specific complementary target nucleic acid molecule in a cell in the subject; allowing the engagement of an endogenous present adenosine deaminating enzyme, such as ADAR1 and/or ADAR2; and allowing the enzyme to deaminate the target adenosine in the target nucleic target molecule to an inosine, thereby alleviating, preventing or ameliorating CVD.

RNA editing molecules present in the cell will usually be proteinaceous in nature, such as the ADAR enzymes found in metazoans, including mammals. Preferably, the cellular editing entity is an enzyme, more preferably an adenosine deaminase or a cytidine deaminase, still more preferably an adenosine deaminase. These are enzymes with ADAR activity. The ones of most interest are the human ADARs, hADAR1 and hADAR2, including any isoforms thereof. RNA editing enzymes known in the art, for which oligonucleotide constructs as disclosed herein may conveniently be designed, include the adenosine deaminases acting on RNA (ADARs), such as hADAR1 and hADAR2 in humans or human cells and cytidine deaminases. It is known that hADAR1 exists in two isoforms; a long 150 kDa interferon inducible version and a shorter, 110 kDa version, that is produced through alternative splicing from a common pre-mRNA. Consequently, the level of the 150 kDa isoform available in the cell may be influenced by interferon, particularly interferon-gamma (IFN-γ). hADAR1 is also inducible by TNF-α. This provides an opportunity to develop combination therapy, whereby IFN-γ or TNF-α and EONs as disclosed herein are administered to a patient either as a combination product, or as separate products, either simultaneously or subsequently, in any order. Certain disease conditions may already coincide with increased IFN-γ or TNF-α levels in certain tissues of a patient, creating further opportunities to make editing more specific for diseased tissues. It will be understood by a person having ordinary skill in the art that the extent to which the editing entities inside the cell are redirected to other target sites may be regulated by varying the affinity of the first nucleic acid strand for the recognition domain of the editing molecule.

### Chemical Modifications

All chemical modifications listed below that may be used in the EON as disclosed herein may also be used for a sense strand that is complementary to the EON, when the EON and the complementary strand form a so-called heteroduplex RNA editing oligonucleotide (HEON) complex, as described in PCT/EP2023/079290 (unpublished), except that the opposite sense strand does not have an orphan nucleotide. Hence, the modification related to the orphan nucleotide relate only to the EON as disclosed herein, but all other modifications relate to the EON as disclosed herein and any (protecting) sense oligonucleotide that may be used together with the EON in a pharmaceutical product. This includes the use of hydrophobic moieties (such as tocopherol and cholesterol) and cell-specific ligands (such as GalNAc moieties), that have also been described herein, and in detail in PCT/EP2023/079290 (unpublished), which may either be bound to the EON or its opposite strand, or both.

The internucleoside linkages in the oligonucleotides as disclosed herein may comprise one or more naturally occurring internucleoside linkages and/or modified internucleoside linkages. Without limitations, at least one, at least two, or at least three internucleoside linkages from a 5′ and/or 3′ end of the EON is preferably a modified internucleoside linkage. A preferred modified internucleoside linkage is a PS linkage. In one aspect, all internucleoside linkages of the EON are modified internucleoside linkages. In one aspect, the EON comprises a PNdmi linkage linking the most terminal nucleoside at the 5′ and/or 3′ end, and the one before last nucleoside at each of these ends, respectively. A PNdmi linkage as preferably used in the EONs as disclosed herein has the structure of the following formula (I):

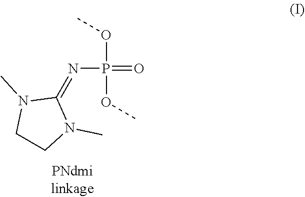

A common limiting factor in oligonucleotide-based therapies are the oligonucleotide's ability to be taken up by the cell (when delivered per se, or ‘naked’ without applying a delivery vehicle), its biodistribution and its resistance to nuclease-mediated breakdown. The skilled person is aware, and it has been described in detail in the art, that a variety of chemical modifications can assist in overcoming such limitations. Examples of such now commonly used chemical modifications are the 2′-O-methyl (often abbreviated to 2′-OMe or 2′-O-Me), 2′-F and 2′-O-methoxyethyl (often also referred to as 2′-methoxyethoxy, or 2′-MOE) modifications of the sugar and the use of PS linkages between nucleosides. WO2020/201406 discloses the use of MP linkage modifications at certain positions surrounding the orphan nucleotide in the first nucleic acid strand. The ribose 2′ groups in all nucleotides of the EON, except for the ribose sugar moiety of the orphan nucleotide that has certain limitations in respect of compatibility with RNA editing, can be independently selected from 2′-H (i.e., DNA), 2′-OH (i.e., RNA), 2′-OMe, 2′-MOE, 2′-F, or 2′-4′-linked (for instance a locked nucleic acid (LNA)), or other ribosyl 1′-substitutions, 2′ substitutions, 3′ substitutions, 4′ substitutions or 5′ substitutions. The orphan nucleotide in the EON that comprises no other chemical modifications to the ribose sugar, the base, or the linkage preferably does not carry a 2′-OMe or 2′-MOE substitution but may carry a 2′-F, a 2′,2′-difluoro (diF), or 2′-ara-F (FANA) substitution or may be DNA. PCT/EP2023/069609 (unpublished) describes the modification of the 2′ position of the ribose sugar moiety of the orphan nucleotide by a 2′,2′-disubstituted substitution such as diF, which is also applicable. The 2′-4′ linkage can be selected from many linkers known in the art, such as a methylene linker, amide linker, or constrained ethyl linker (cEt).

The disclosure provides an EON for use in the deamination of a target nucleotide (preferably adenosine) in a target RNA, wherein the EON is complementary to a stretch of nucleotides in the target RNA that includes the target adenosine, wherein the nucleotide in the first nucleic acid strand that is directly opposite the target nucleotide is the orphan nucleotide, and when the target nucleotide is an adenosine the orphan nucleotide comprises preferably a base or modified base or base analogue with a NH moiety at the position similar to the ring nitrogen (e.g., Benner's base Z). The nucleotide numbering in the EON is such that the orphan nucleotide is number 0 and the nucleotide 5′ from the orphan nucleotide is number +1. Counting is further positively (+) incremented towards the 5′ end and negatively (−) incremented towards the 3′ end, wherein the first nucleotide 3′ from the orphan nucleotide is number −1. The internucleoside linkage numbering in the EON is such that linkage number 0 is the linkage 5′ from the orphan nucleotide, and the linkage positions in the oligonucleotide are positively (+) incremented towards the 5′ end and negatively (−) incremented towards the 3′ end.

Preferably, the EON comprises one or more (chirally pure or chirally mixed) PS linkages. In one aspect, the PS linkages connect the terminal 3, 4, 5, 6, 7, or 8 nucleotides on each end of the first nucleic acid strand. In one aspect, the EON comprises one of more phosphoramidate (PN) linkages. In one aspect, a PN linkage connects the terminal two nucleotides on each end of the EON as disclosed herein.

A nucleoside in the EON may be a natural nucleoside (deoxyribonucleoside or ribonucleoside) or a non-natural nucleoside. It is noted that for RNA editing, in which double-stranded RNA is generally the substrate for enzymes with deamination activity (such as ADARs), ribonucleosides are considered ‘natural’, while deoxyribonucleosides may then be, for the sake of argument, considered as non-natural, or modified, simply because DNA is not present in the RNA-RNA double stranded substrate configurations. The skilled person appreciates that when the nucleotide has a natural ribose moiety, it may still be non-naturally modified in the base and/or the linkage.

In addition to the specific preferred chemical modifications at certain positions in compounds as disclosed herein, compounds as disclosed herein may comprise one or more (additional) modifications to the nucleobase, scaffold and/or backbone linkage, which may or may not be present in the same monomer, for instance at the 3′ and/or 5′ position. A scaffold modification indicates the presence of a modified version of the ribosyl moiety as naturally occurring in RNA (i.e., the pentose moiety), such as bicyclic sugars, tetrahydropyrans, hexoses, morpholinos, 2′-modified sugars, 4′-modified sugar, 5′-modified sugars and 4′-substituted sugars. Examples of suitable modifications include, but are not limited to 2′-O-modified RNA monomers, such as 2′-O-alkyl or 2′-O-(substituted)alkyl such as 2′-OMe, 2′-O-(2-cyanoethyl), 2′-MOE, 2′-O-(2-thiomethyl)ethyl, 2′-O-butyryl, 2′-O-propargyl, 2′-O-allyl, 2′-O-(2-aminopropyl), 2′-O-(2-(dimethylamino) propyl), 2′-O-(2-amino)ethyl, 2′-O-(2-(dimethylamino)ethyl); 2′-deoxy (DNA); 2′-O-(haloalkyl)methyl such as 2′-O-(2-chloroethoxy)methyl (MCEM), 2′-O-(2,2-dichloroethoxy)methyl (DCEM); 2′-O-alkoxycarbonyl such as 2′-O-[2-(methoxycarbonyl)ethyl] (MOCE), 2′-O-[2-N-methylcarbamoyl)ethyl] (MCE), 2′-O-[2-(N,N-dimethylcarbamoyl)ethyl] (DCME); 2′-halo e.g. 2′-F, FANA; 2′-O-[2-(methylamino)-2-oxoethyl] (NMA); a bicyclic or bridged nucleic acid (BNA) scaffold modification such as a conformationally restricted nucleotide (CRN) monomer, a locked nucleic acid (LNA) monomer, a xylo-LNA monomer, an α-LNA monomer, an α-I-LNA monomer, a β-d-LNA monomer, a 2′-amino-LNA monomer, a 2′-(alkylamino)-LNA monomer, a 2′-(acylamino)-LNA monomer, a 2′-N-substituted 2′-amino-LNA monomer, a 2′-thio-LNA monomer, a (2′-0,4′-C) constrained ethyl (cEt) BNA monomer, a (2′-0,4′-C) constrained methoxyethyl (cMOE) BNA monomer, a 2′,4′-BNA^{NC}(NH) monomer, a 2′,4′-BNA^{NC}(NMe) monomer, a 2′,4′-BNA^{NC}(NBn) monomer, an ethylene-bridged nucleic acid (ENA) monomer, a carba-LNA (cLNA) monomer, a 3,4-dihydro-2H-pyran nucleic acid (DpNA) monomer, a 2′-C-bridged bicyclic nucleotide (CBBN) monomer, an oxo-CBBN monomer, a heterocyclic-bridged BNA monomer (such as triazolyl or tetrazolyl-linked), an amido-bridged BNA monomer (such as AmNA), an urea-bridged BNA monomer, a sulfonamide-bridged BNA monomer, a bicyclic carbocyclic nucleotide monomer, a TriNA monomer, an α-I-TriNA monomer, a bicyclo DNA (bcDNA) monomer, an F-bcDNA monomer, a tricyclo DNA (tcDNA) monomer, an F-tcDNA monomer, an alpha anomeric bicyclo DNA (abcDNA) monomer, an oxetane nucleotide monomer, a locked PMO monomer derived from 2′-amino LNA, a guanidine-bridged nucleic acid (GuNA) monomer, a spirocyclopropylene-bridged nucleic acid (scpBNA) monomer, and derivatives thereof; cyclohexenyl nucleic acid (CeNA) monomer, altriol nucleic acid (ANA) monomer, hexitol nucleic acid (HNA) monomer, fluorinated HNA (F-HNA) monomer, pyranosyl-RNA (p-RNA) monomer, 3′-deoxypyranosyl DNA (p-DNA), unlocked nucleic acid UNA); an inverted version of any of the monomers above. All these modifications are known to the person skilled in the art.

The base sequence of the EON disclosed herein is complementary to part of the base sequence of a target B4GALT1 transcription product that includes at least a target adenosine (preferably the adenosine at position 1055) that is to be deaminated to an inosine, and therefore can anneal (or hybridize) to the target transcription product. The complementarity of a base sequence can be determined by using a BLAST program or the like. Those skilled in the art can easily determine the conditions (temperature, salt concentration, and the like) under which two strands can be hybridized, taking into consideration the complementarity between the strands.

The EON as disclosed herein, in contrast to what has been described for gapmers and their relation towards RNase breakdown and the use of such gapmers in double-stranded complexes (see for instance EP 3954395 A1), does not comprise a stretch of DNA nucleotides which would make a target sequence (or a sense nucleic acid strand) a target for RNase-mediated breakdown. In one aspect, the EON as disclosed herein does not comprise four or more consecutive DNA nucleotides anywhere within its sequence. In one aspect, the EON as disclosed herein is composed of as much (chemically) modified nucleotides as possible to enhance the resistance towards RNase-mediated breakdown, while at the same time being as efficient as possible in producing an RNA editing effect. This means that the orphan nucleotide and several other nucleotides within the EON may be DNA, but also that there is no stretch of four or more consecutive DNA nucleotides within the EON. Hence, the EON as disclosed herein is not a gapmer. A gapmer is in principle a single-stranded nucleic acid consisting of a central region (DNA gap region with at least four consecutive deoxyribonucleotides) and wing regions positioned directly at the 5′ end (5′ wing region) and the 3′ end (3′ wing region) thereof. In contrast, the EON as disclosed herein may be any oligonucleotide that produces, causes, triggers, allows an RNA editing effect in which a target adenosine in a target RNA molecule is deaminated to an inosine, and accordingly is resistant to RNase-mediated breakdown as much as possible to yield this effect.

In one aspect, the EON as disclosed herein, or the sense strand to which it may be annealed before entering a target cell, is bound to a hydrophobic moiety, such as palmityl or an analog thereof, cholesterol or analog thereof, or tocopherol or analog thereof. It is preferably bound to the 5′ terminus. In case a hydrophobic moiety is bound to the 5′ terminus as well as to the 3′ terminus, such hydrophobic moieties may the same or different. The hydrophobic moiety bound to the oligonucleotide may be bound directly, or indirectly mediated by another substance. When the hydrophobic moiety is bound directly, it is sufficient if the moiety is bound via a covalent bond, an ionic bond, a hydrogen bond, or the like. When the hydrophobic moiety is bound indirectly, it may be bound via a linking group (a linker). The linker may be a cleavable or an uncleavable linker. A cleavable linker refers to a linker that can be cleaved under physiological conditions, for example, in a cell or an animal body (e.g., a human body). A cleavable linker is selectively cleaved by an endogenous enzyme such as a nuclease, or by physiological circumstances specific to parts of the body or cell, such as pH or reducing environment (such as glutathione concentrations). Examples of a cleavable linker comprise, but is not limited to, an amide, an ester, one or both esters of a phosphodiester, a phosphoester, a carbamate, and a disulfide bond, as well as a natural DNA linker. Cleavable linkers also include self-immolative linkers. An uncleavable linker refers to a linker that is not cleaved under physiological conditions, or very slowly compared to a cleavable linker, for example, in a PS linkage, modified or unmodified deoxyribonucleosides linked by a PS linkage, a spacer connected through a PS bond and a linker consisting of modified or unmodified ribonucleosides. There is no restriction on the chain length, when a linker is a nucleic acid such as DNA, or an oligonucleotide. However, it may be usually from 2 to 20 bases in length, from 3 to 10 bases in length, or from 4 to 6 bases in length. There is no restriction on the length or composition of a spacer that is connects the ligand and the oligonucleotide, and may include for example ethylene glycol, TEG, HEG, alkyl chains, propyl, 6-aminohexyl, or dodecyl.

The disclosure also provides a pharmaceutical composition comprising the EON as disclosed herein, and further comprising a pharmaceutically acceptable carrier and/or other additive and may be dissolved in a pharmaceutically acceptable organic solvent, or the like. Dosage forms in which the EON or the pharmaceutical composition are administered may depend on the disorder to be treated and the tissue that needs to be targeted and can be selected according to common procedures in the art. The pharmaceutical compositions may be administered by a single-dose administration or by multiple dose administration. It may be administered daily or at appropriate time intervals, which may be determined using common general knowledge in the field and may be adjusted based on the disorder and the efficacy of the active ingredient.

In one aspect, the EON as disclosed herein comprises at least one nucleotide with a sugar moiety that comprises a 2′-OMe modification. In one aspect, the EON as disclosed herein comprises at least one nucleotide with a sugar moiety that comprises a 2′-MOE modification. In one aspect, the EON as disclosed herein comprises at least one nucleotide with a sugar moiety that comprises a 2′-F modification. In one aspect, the orphan nucleotide carries a 2′-H in the sugar moiety and is therefore referred to as a DNA nucleotide, even though additional modifications may exist in its base and/or linkage to its neighbouring nucleosides. In one aspect, the orphan nucleotide carries a 2′-F in the sugar moiety. In one aspect, the orphan nucleotide carries a diF substitution in the sugar moiety. In one aspect, the orphan nucleotide carries a 2′-F and a 2′-C-methyl in the sugar moiety. In one aspect, the orphan nucleotide comprises a 2′-F in the arabinose configuration (FANA) in the sugar moiety. In one aspect, the EON is an antisense oligonucleotide that can form a double stranded nucleic acid complex with a target RNA molecule, wherein the double stranded nucleic acid complex can recruit an adenosine deaminating enzyme for deamination of a target adenosine in the target B4GALT1 RNA molecule, wherein the nucleotide in the EON that is opposite the target adenosine is the orphan nucleotide, and wherein the orphan nucleotide has the following formula (II):

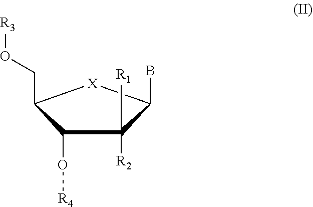

wherein: X is O, NH, OCH₂, CH₂, Se, or S; B is a nitrogenous base selected from the group consisting of: cytosine, uracil, isouracil, N3-glycosylated uracil, pseudoisocytosine, 8-oxo-adenine, and 6-amino-5-nitro-3-yl-2 (1H)-pyridone; R₁ and R₂ are both selected, independently, from H, OH, F or CH₃; R₃ is the part of the EON that is 5′ of the orphan nucleotide, consisting of 7 to 30 nucleotides; and R₄ is the part of the EON that is 3′ of the orphan nucleotide, consisting of 4 to 25 nucleotides. The nucleotide 3′ and/or 5′ from the orphan nucleotide may be DNA, more preferably the nucleotide at the 3′ (position-1).

In one aspect, the EON as disclosed herein comprises at least one MP internucleoside linkage according to the following formula (III):

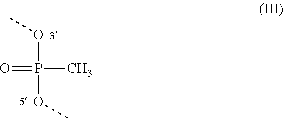

A preferred position for an MP linkage in an EON as disclosed herein is linkage-2, thereby connecting the nucleoside at position-1 with the nucleoside at position-2, although other positions for MP linkages are not explicitly excluded.

An EON as disclosed herein may also comprise one or more linkage modifications according to the structure of the following formula (IV):

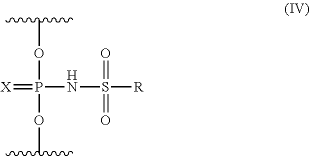

wherein:
X=O or S; and
R=an aryl, a substituted aryl, a heterocycle, a substituted heterocycle, an aromatic heterocycle, a substituted aromatic heterocycle, a C₁-C₆ alkoxy, a substituted C₁-C₆ alkoxy, a C₁-C₂₀ alkyl, a substituted C₁-C₂₀ alkyl, a C₁-C₆ alkenyl, a C₁-C₆ substituted alkenyl, a C₁-C₆ alkynyl, a substituted C₁-C₆ alkynyl, or a conjugate group. In a preferred embodiment, X=O and R=methyl and the linkage modification is referred to as mesyl phosphoramidate, MsPA or PNms. In a preferred aspect, the PN mesyl (or PNms) linkage is at linkage position-2 in the EON as disclosed herein (where it is then present instead of a PS, PO, or MP linkage), such as in EON B4GALT1-65 (SEQ ID NO: 1079).

In a preferred aspect, the EON as disclosed herein comprises an internucleoside linkage of the structure of formula (IV), wherein X=O and R=CH₃, which linkage is generally referred to herein as a PNms linkage (mesyl phosphoramidate). In other preferred aspects, R equals one of the following structures (a), (b), (c), (d), (e), (f), (g), (h), or (i):

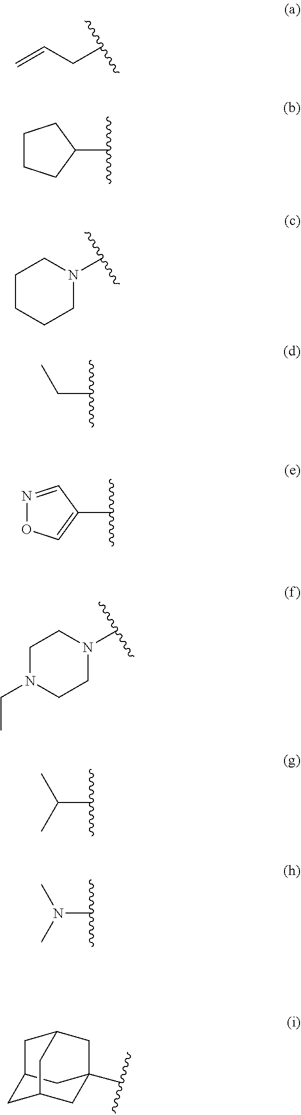

Disclosed herein is also an EON that is able to mediate adenosine deamination by recruitment of a deaminating enzyme in a cell after the EON has formed a double-stranded complex with a region of a target RNA nucleic acid molecule in a cell, wherein the region comprises a target adenosine, wherein the deaminating enzyme can deaminate the target adenosine into an inosine, and wherein the EON comprises a moiety with a structure according to formula (V):

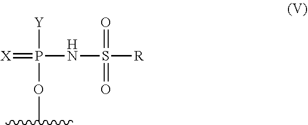

wherein: X=O or S;
Y=O⁻ or S⁻; and
R=an aryl, a substituted aryl, a heterocycle, a substituted heterocycle, an aromatic heterocycle, a substituted aromatic heterocycle, a C₁-C₆ alkoxy, a substituted C₁-C₆ alkoxy, a C₁-C₂₀ alkyl, a substituted C₁-C₂₀ alkyl, a C₁-C₆ alkenyl, a C₁-C₆ substituted alkenyl, a C₁-C₆ alkynyl, a substituted C₁-C₆ alkynyl, or a conjugate group. In a preferred embodiment, X=O and R=methyl.

An EON as disclosed herein may comprise a substitution of one of the non-bridging oxygens in the phosphodiester linkage. This modification slightly destabilizes base pairing but adds significant resistance to nuclease degradation. A preferred nucleotide analogue or equivalent comprises PS, phosphonoacetate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate. Particularly preferred are internucleoside linkages that are modified to contain a PS. Particularly preferred are internucleoside linkages that are modified to contain an MP. Particularly preferred are internucleoside linkages that are modified to contain a PNms. Particularly preferred are internucleoside linkages that are modified to contain a PNdmi. The regular internucleosidic linkages between the nucleotides may be altered by mono- or di-thioation of the phosphodiester bonds to yield PS esters or phosphorodithioate esters, respectively. Other modifications of the internucleosidic linkages are possible, including amidation and peptide linkers. The skilled person can determine for what target RNA nucleic acid molecule the EON comprises a certain linkage modification at each linkage position of the EON as disclosed herein to generate the most effective and most stable oligonucleotide compound.

In one aspect, the EON as disclosed herein comprises at least one nucleotide with a sugar moiety that comprises a 2′-fluoro (2′-F) modification. A preferred position for the nucleotide that carries a 2′-F modification is position-3 in the EON, which may be present together with an identical 2′ modification in the orphan nucleotide as discussed above.

In one aspect, the EON as disclosed herein comprises at least one phosphonoacetate or phosphonoacetamide internucleoside linkage.

In one aspect, the EON as disclosed herein comprises at least one nucleotide comprising an LNA ribose modification, or a UNA ribose modification. In one aspect, the EON as disclosed herein comprises at least one nucleotide comprising a TNA ribose modification.

The skilled person knows that an oligonucleotide, such as an EON as outlined herein, generally consists of repeating monomers. Such a monomer is most often a nucleotide or a chemically modified nucleotide. The most common naturally occurring nucleotides in RNA are adenosine monophosphate (A), cytidine monophosphate (C), guanosine monophosphate (G), and uridine monophosphate (U). These consist of a pentose sugar, a ribose, a 5′-linked phosphate group which is linked via a phosphate ester, and a 1′-linked base. The sugar connects the base and the phosphate and is therefore often referred to as the “scaffold” of the nucleotide.

A modification in the pentose sugar is therefore often referred to as a ‘scaffold modification’. The original pentose sugar may be replaced in its entirety by another moiety that similarly connects the base and the phosphate. It is therefore understood that while a pentose sugar is often a scaffold, a scaffold is not necessarily a pentose sugar. Examples of scaffold modifications that may be applied in the monomers of the EON as disclosed herein are disclosed in WO2020/154342, WO2020/154343, and WO2020/154344.

In one aspect, the EON as disclosed herein may comprise one or more nucleotides carrying a 2′-MOE ribose modification. Also, in one aspect, the EON as disclosed herein comprises one or more nucleotides not carrying a 2′-MOE ribose modification, and wherein the 2′-MOE ribose modifications are at positions that do not prevent the enzyme with adenosine deaminase activity from deaminating the target adenosine. In another aspect, the EON as disclosed herein comprises 2′-OMe ribose modifications at the positions that do not comprise a 2′-MOE ribose modification, and/or wherein the EON comprises deoxynucleotides at positions that do not comprise a 2′-MOE ribose modification. In one aspect the EON as disclosed herein comprises one or more nucleotides comprising a 2′ position comprising a 2′-MOE, 2′-OMe, 2′-OH, 2′-deoxy, TNA, 2′-fluoro (2′-F), a 2′,2′-disubstituted modification (such as a 2′,2′-difluoro (diF) modification, a 2′-fluoro-2′-C-methyl modification, or others such as those indicated in e.g., Grosse et al. (2022. ACS Med Chem Lett DOI: 10.1021/acsmedchemlett.2c00372) including 2′-spirocyclic ones) or a 2′-4′-linkage (i.e., a bridged nucleic acid such as a LNA or examples mentioned in e.g., WO2018/007475)). In another aspect, other nucleic acid monomer that are applied are arabinonucleic acids and 2′-deoxy-2′-fluoroarabinonucleic acid (FANA), for instance for improved affinity purposes. The 2′-4′ linkage can be selected from linkers known in the art, such as a methylene linker or constrained ethyl linker. A wide variety of 2′ modifications are known in the art. Further examples are disclosed in further detail in WO2016/097212, WO2017/220751, WO2018/041973, WO2018/134301, WO2019/219581, WO2019/158475, and WO2022/099159 for instance. In all cases, the modifications should be compatible with editing such that the EON fulfils its role as an editing triggering oligonucleotide that can form a double stranded complex with the target RNA and recruit a deaminating enzyme, which enzyme can subsequently deaminate the target adenosine. Where a monomer comprises a UNA ribose modification, that monomer can have a 2′ position comprising the same modifications discussed above, such as a 2′-MOE, a 2′-OMe, a 2′-OH, a 2′-deoxy, a 2′-F, a 2′,2′-diF, a 2′-fluoro-2′-C-methyl, an arabinonucleic acid, a FANA, or a 2′-4′-linkage (i.e., a bridged nucleic acids such as an LNA).

A base, sometimes called a nucleobase, is generally adenine, cytosine, guanine, thymine or uracil, or a derivative thereof. A base, sometimes called a nucleobase, is defined as a moiety that can bond to another nucleobase through H-bonds, polarized bonds (such as through CF moieties) or aromatic electronic interactions. Cytosine, thymine, and uracil are pyrimidine bases, and are generally linked to the scaffold through their 1-nitrogen. Adenine and guanine are purine bases and are generally linked to the scaffold through their 9-nitrogen. The terms ‘adenine’, ‘guanine’, ‘cytosine’, ‘thymine’, ‘uracil’ and ‘hypoxanthine’ as used herein refer to the nucleobases as such. The terms ‘adenosine’, ‘guanosine’, ‘cytidine’, ‘thymidine’, ‘uridine’ and ‘inosine’ refer to the nucleobases linked to the (deoxy) ribosyl sugar.

The nucleobases in an EON as disclosed herein can be adenine, cytosine, guanine, thymine, inosine, or uracil or any other moiety able to interact with another nucleobase through H-bonds, polarized bonds (such as CF) or aromatic electronic interactions. The nucleobases at any position in the nucleic acid strand can be a modified form of adenine, cytosine, guanine, or uracil, such as hypoxanthine (the nucleobase in inosine), pseudouracil, pseudocytosine, iso-uracil, N3-glycosylated uracil, 1-methylpseudouracil, orotic acid, agmatidine, lysidine, 2-thiouracil, 2-thiothymine, 5-substituted pyrimidine (e.g., 5-halouracil, 5-halomethyluracil, 5-trifluoromethyluracil, 5-propynyluracil, 5-propynylcytosine, 5-aminomethyluracil, 5-hydroxymethyluracil, 5-formyluracil, 5-aminomethylcytosine, 5-formylcytosine), 5-hydroxymethylcytosine, 7-deazaguanine, 7-deazaadenine, 7-deaza-2,6-diaminopurine, 8-aza-7-deazaguanine, 8-aza-7-deazaadenine, 8-aza-7-deaza-2,6-diaminopurine, 8-oxo-adenine, 3-deazapurine (such as a 3-deaza-adenosine), pseudoisocytosine, N4-ethylcytosine, N2-cyclopentylguanine, N2-cyclopentyl-2-aminopurine, N2-propyl-2-aminopurine, 2,6-diaminopurine, 2-aminopurine, G-clamp and its derivatives, Super A, Super T, Super G, amino-modified nucleobases or derivatives thereof; and degenerate or universal bases, like 2,6-difluorotoluene, or absent like abasic sites (e.g., 1-deoxyribose, 1,2-dideoxyribose, 1-deoxy-2-O-methylribose, and azaribose).

In one aspect, the nucleotide analog is an analog of a nucleic acid nucleotide. In one aspect, the nucleotide analog is an analog of adenosine, guanosine, cytidine, thymidine, uridine, deoxyadenosine, deoxyguanosine, deoxycytidine, deoxythymidine, or deoxyuridine. In one aspect, the nucleotide analog is not guanosine or deoxyguanosine. In one aspect, the nucleotide analog is not a nucleic acid nucleotide. In one aspect, the nucleotide analog is not adenosine, guanosine, cytidine, thymidine, uridine, deoxyadenosine, deoxyguanosine, deoxycytidine, deoxythymidine, or deoxyuridine.

A nucleotide is generally connected to neighboring nucleotides through condensation of its 5′-phosphate moiety to the 3′-hydroxyl moiety of the neighboring nucleotide monomer. Similarly, its 3′-hydroxyl moiety is generally connected to the 5′-phosphate of a neighboring nucleotide monomer. This forms phosphodiester bonds. The phosphodiesters and the scaffold form an alternating copolymer. The bases are grafted on this copolymer, namely to the scaffold moieties. Because of this characteristic, the alternating copolymer formed by linked scaffolds of an oligonucleotide is often called the ‘backbone’ of the oligonucleotide. Because phosphodiester bonds connect neighboring monomers together, they are often referred to as ‘backbone linkages’. It is understood that when a phosphate group is modified so that it is instead an analogous moiety such as a PS, such a moiety is still referred to as the backbone linkage of the monomer. This is referred to as a ‘backbone linkage modification’. In general terms, the backbone of an oligonucleotide comprises alternating scaffolds and backbone linkages.

EONs as disclosed herein can comprise linkage modifications. A linkage modification can be, but not limited to, a modified version of the phosphodiester present in RNA, such as PS, chirally pure PS, (R)-PS, (S)-PS, MP, chirally pure MP, (R)-methyl phosphonate, (S)-methyl phosphonate, phosphoryl guanidine (such as PNdmi), chirally pure phosphoryl guanidine, (R)-phosphoryl guanidine, (S)-phosphoryl guanidine, phosphorodithioate (PS2), phosphonacetate (PACE), phosphonoacetamide (PACA), thiophosphonoacetate, thiophosphonoacetamide, methyl phosphorohioate, methyl thiophosphonate, PS prodrug, alkylated PS, H-phosphonate, ethyl phosphate, ethyl PS, boranophosphate, borano PS, methyl boranophosphate, methyl borano PS, methyl boranophosphonate, methyl boranophosphothioate, phosphate, phosphotriester, aminoalkylphosphotriester, and their derivatives. Another modification includes phosphoramidite, phosphoramidate, N3′→P5′ phosphoramidate, phosphorodiamidate, phosphorothiodiamidate, sulfamate, diethylenesulfoxide, amide, sulfonate, siloxane, sulfide, sulfone, formacetyl, alkenyl, methylenehydrazino, sulfonamide, triazole, oxalyl, carbamate, methyleneimino (MMI), and thioacetamide nucleic acid (TANA); and their derivatives. Various salts, mixed salts and free acid forms are also included, as well as 3′→3′ and 2′→5′ linkages.

In one aspect, an EON as disclosed herein comprises a substitution of one of the non-bridging oxygens in the phosphodiester linkage. This modification slightly destabilizes base-pairing but adds significant resistance to nuclease degradation. A preferred nucleotide analogue or equivalent comprises PS, phosphonoacetate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate. Particularly preferred are internucleoside linkages that are modified to contain a PS. Many of these non-naturally occurring modifications of the linkage, such as PS are chiral, which means that there are Rp and Sp configurations, known to the person skilled in the art. In one aspect, the chirality of the PS linkages is controlled, which means that each of the linkages is either in the Rp or in the Sp configuration, whichever is preferred. The choice of an Rp or Sp configuration at a specified linkage position may depend on the target sequence, the EON sequence, and the efficiency of binding and induction of providing RNA editing. However, if such is not specifically desired, a composition may comprise EONs as active compounds with both Rp and Sp configurations at a certain specified linkage position. Mixtures of such EONs are also feasible, wherein certain positions have preferably either one of the configurations, while for other positions such does not matter.

Again, in all cases, the modifications should be compatible with editing such that the EON fulfils its role as an editing producing oligonucleotide that can, when attached to its target sequence recruit an adenosine deaminase enzyme because of the dsRNA nature that arises. In all aspects, the enzyme with adenosine deaminase activity is preferably ADAR1 or ADAR2. In a highly preferred aspect, the EON is an RNA editing oligonucleotide that targets a pre-mRNA or an mRNA, wherein the target nucleotide is an adenosine in the target RNA, wherein the adenosine is deaminated to an inosine, which is being read as a guanosine by the translation machinery. The disclosure also provides a pharmaceutical composition comprising the EON as characterized herein, and a pharmaceutically acceptable carrier.

Other chemical modifications of the EON as disclosed herein include the substitution of one or more than one of any of the hydrogen atoms with deuterium or tritium, examples of which can be found in e.g., WO2014/022566 or WO2015/011694.

The disclosure also provides an EON as disclosed herein, or a pharmaceutical composition comprising an EON as disclosed herein, for use in the treatment, prevention, or amelioration of CVD, such as CAD. In one aspect, the disclosure provides an EON, or a pharmaceutical composition comprising an EON as disclosed herein, for use in the treatment, prevention, or amelioration of a disease wherein B4GALT1 functions in a wildtype manner. In one aspect, the disclosure provides an EON, or a pharmaceutical composition comprising an EON as disclosed herein, for use in the treatment, prevention or amelioration of a disease related to high LDL-C and/or fibrinogen levels. In one aspect, the disclosure provides an EON, or a pharmaceutical composition comprising an EON as disclosed herein, for use in the treatment or prevention of CVD, such as CAD.

EONs as disclosed herein preferably do not include a 5′-terminal 06-benzylguanosine or a 5′-terminal amino modification and preferably are not covalently linked to a SNAP-tag domain (an engineered 06-alkylguanosine-DNA-alkyl transferase). EONs as disclosed herein preferably do not comprise a boxB RNA hairpin sequence. In one aspect, an EON as disclosed herein comprises 0, 1, 2 or 3 wobble base pairs with the target sequence, and/or 0, 1, 2, 3, 4, 5, 6, 7, or 8 mismatching base pairs with the target RNA sequence. No mismatch may exist when the orphan nucleotide is uridine. One alternative for uridine is positioning an iso-uridine opposite the target adenosine, which does not pair like U pairs with A, and is therefore considered as a mismatch. Preferably, the target adenosine in the target sequence forms a mismatch base pair with the nucleoside in the EON that is directly opposite the target adenosine.

It should be noted that when an EON is delivered through a vector, for instance an AAV vector, chemical modifications are not present in the EON that acts on the target RNA molecule. Although it is preferred to use ‘naked’ EONs that have chemical modifications as outlined herein, EONs that are delivered through other means, for instance through AAV vector expression, or editing molecules that are circular, or have hairpin structures (recruiting portions, e.g., as disclosed in WO2016/097212, WO2017/050306, WO2020/001793, WO2017/010556, WO2020/246560, and WO2022/078995) are also encompassed by the present disclosure because these can also be applied to edit adenosines in the target B4GALT1 RNA molecule to generate a B4GALT1 protein with reduced function.

Notably, when the EON comprises chemical modifications, as detailed herein, it may still be delivered through the means of a delivery vehicle. Suitable delivery vehicles are nanoparticle delivery vehicles such as polymeric nanoparticles, dendrimers, inorganic nanoparticles and nanocrystals, organic nanocrystals, and liposomes. Preferred nanoparticles are Lipid Nanoparticles (LNP's) that are nano-sized lipid vesicles that carry the EON of the present disclosure and aid to the delivery of target cells. In the event that an LNP is applied or any other similar type of carrier, the EON is still considered naked because it is not transcribed from an encoding polynucleotide (such as in the case of a plasmid or a vector, in which the EON is not regarded as ‘naked’). So, even though a chemically modified EON is encapsulated by a carrier, preferably an LNP, it is still seen as naked, as it has been manufactured as such in a laboratory setting and encapsulated thereafter in the carrier using methods known to the person skilled in the art. The disclosure also relates to a delivery vehicle, preferably an LNP, which comprises a ‘naked’ and chemically modified EON as disclosed herein, even more preferably as disclosed in any one of SEQ ID NO:3 to 42, 59 to 1069, and 1078 to 1190, preferably from the group consisting of SEQ ID NO:23, 19, 31, 27, 35, 39, 69, 70, 71, 72, 73, 93, 94, 95, 1079, 1084, 1093, 1095, 1100, 1102, 1115, 1121, 1123, 1124, and 1139 to 1190. The person skilled in the art understands that when a delivery moiety, or attachment to the EON is used (such a GalNAc moiety to target hepatocytes in the liver) that the EON is still seen as naked as well, also when a GalNAc-(linker)-EON is encapsulated in a delivery vehicle such as an LNP.

An EON as disclosed herein can utilise endogenous cellular pathways and naturally available ADAR enzymes (=endogenous) to specifically edit a target adenosine in the target RNA sequence. An EON as disclosed herein can recruit endogenous ADAR and complex with it when present in a double-stranded complex with the target RNA molecule, and then facilitates the deamination of a (single) specific target adenosine nucleotide in a target RNA sequence. Ideally, only one adenosine is deaminated. An EON as disclosed herein, when complexed to ADAR, preferably brings about the deamination of a single target adenosine.

Analysis of natural targets of ADAR enzymes has indicated that these generally include mismatches between the two strands that form the RNA helix edited by ADAR1 or ADAR2. It has been suggested that these mismatches enhance the specificity of the editing reaction (Stefl et al. 2006. Structure 14 (2): 345-355; Tian et al. 2011. Nucleic Acid Res 39 (13): 5669-5681). Characterization of optimal patterns of paired/mismatched nucleotides between the EONs and the target RNA also appears important to the development of efficient ADAR-based EON therapy.

As outlined above, an EON as disclosed herein makes use of specific nucleotide modifications at predefined spots to ensure stability as well as proper ADAR binding and activity. These changes may vary and may include modifications in the backbone of the EON, in the sugar moiety of the nucleotides as well as in the nucleobases or the phosphodiester linkages, as outlined in detail herein. They may also be variably distributed throughout the sequence of the EON. Specific modifications may be needed to support interactions of different amino acid residues within the RNA-binding domains of ADAR enzymes, as well as those in the deaminase domain. For example, PS linkages between nucleotides or 2′-OMe or 2′-MOE modifications may be tolerated in some parts of the EON, while in other parts they should be avoided so as not to disrupt crucial interactions of the enzyme with the phosphate and 2′-OH groups. Specific nucleotide modifications may also be necessary to enhance the editing activity on substrate RNAs where the target sequence is not optimal for ADAR editing. Previous work has established that certain sequence contexts are more amenable to editing. For example, a target sequence 5′-UAG-3′ (with the target A in the middle) contains the most preferred nearest-neighbor nucleotides for ADAR2, whereas a 5′-CAA-3′ target sequence is disfavored (Schneider et al. 2014. Nucleic Acids Res 42 (10): e87). The structural analysis of ADAR2 deaminase domain hints at the possibility of enhancing editing by careful selection of the nucleotides that are opposite to the target trinucleotide. For example, the 5′-CAA-3′ target sequence, paired to a 3′-GCU-5′ sequence on the opposing strand (with the A-C mismatch formed in the middle), is disfavored because the guanosine base sterically clashes with an amino acid side chain of ADAR2. Although other adenosines in the B4GALT1 transcript may be targeted to impair the protein function, in a preferred aspect, the adenosine at position 1055 is deaminated. The disclosure also provides RNA editing oligonucleotides, generally referred to as EONs herein, that can bring about deamination of an adenosine in the B4GALT1 transcript, with a resulting B4GALT1 enzyme that has a reduced turnover rate. This means that it is not strictly limited to deamination of the adenosine at position 1055, but that other (single or multiple) adenosines may be targeted, which may also result in decreased B4GALT1 enzyme function. Other adenosines may be identified, for instance by genetic screening in the population, or in silico, that are also important for B4GALT1 function, and that also may be targeted through RNA editing, following the teaching of the present disclosure. All such RNA events and oligonucleotides that can be used for such targeting are encompassed by the present disclosure, no matter what the exact nucleic molecule, or EON, looks like.

Mutagenesis studies of human ADAR2 revealed that a single mutation at residue 488 from glutamate to glutamine (E488Q), gave an increase in the rate constant of deamination by 60-fold when compared to the wild-type enzyme (Kuttan & Bass. 2012. Proc Natl Acad Sci USA. 109 (48): 3295-3304). During the deamination reaction, ADAR flips the edited base out of its RNA duplex, and into the enzyme active site (Matthews et al. 2016. Nat Struct Mol Biol. 23 (5): 426-433). When ADAR2 edits adenosines in the preferred context (an A: C mismatch) the nucleotide opposite the target adenosine is referred to as the orphan cytidine. The crystal structure of ADAR2 E488Q bound to dsRNA revealed that the glutamine (Gln) side chain at position 488 can donate an H-bond to the N3 position of the orphan cytidine, which leads to the increased catalytic rate of ADAR2 E488Q. In the wild-type enzyme, wherein a glutamate (Glu) is present at position 488 instead of a glutamine (Gln) the amide group of the glutamine is absent and is instead a carboxylic acid. To obtain the same contact of the orphan cytidine with the E488Q mutant would then, for the wild-type situation, require protonation for this contact to occur. To make use of endogenously expressed ADAR2 to correct disease relevant mutations, it is essential to maximize the editing efficiency of the wild type ADAR2 enzyme present in the cell. WO2020/252376 discloses the use of EONs with modified RNA bases, especially at the position of the orphan cytidine to mimic the hydrogen-bonding pattern observed by the E488Q ADAR2 mutant. By replacing the nucleotide opposite the target adenosine in the EON with cytidine analogs that serve as H-bond donors at N3, it was envisioned that it would be possible to stabilize the same contact that is believed to provide the increase in catalytic rate for the mutant enzyme. Two cytidine analogs were of particular interest: pseudoisocytidine (also referred to as ‘piC’; Lu et al. 2009. J Org Chem. 74 (21): 8021-8030; Burchenal et al. 1976. Cancer Res 36:1520-1523) and Benner's base Z (also referred to as ‘Zd’; Yang et al. 2006. Nucl Acid Res. 34 (21): 6095-6101) that were initially selected because they offer hydrogen-bond donation at N3 with minimal perturbation to the shape of the nucleobase. Benner's base is also referred to as 6-amino-5-nitro-3-yl-2 (1H)-pyridone. The presence of the cytidine analog in the EON may exist in addition to modifications to the ribose 2′ group. The ribose 2′ groups in the EON can be independently selected from 2′-H (i.e., DNA), 2′-OH (i.e., RNA), 2′-OMe, 2′-MOE, 2′-F, or 2′-4′-linked (i.e., a bridged nucleic acid such as an LNA), or other 2′ substitutions. The 2′-4′ linkage can be selected from linkers known in the art, such as a methylene linker or constrained ethyl linker.

In one aspect, an EON comprises one or more sugar moieties that are mono- or di-substituted at the 2′, 3′ and/or 5′ position such as: —OH; -H; -F; substituted or unsubstituted, linear or branched lower (C₁-C₁₀) alkyl, alkenyl, alkynyl, alkaryl, allyl, or aralkyl, that may be interrupted by one or more heteroatoms; —O-, S-, or N-alkyl; —O-, S-, or N-alkenyl; —O-, S-, or N-alkynyl; —O—, S-, or N-allyl; —O-alkyl-O-alkyl; -methoxy; -aminopropoxy; -methoxyethoxy; -dimethylamino oxyethoxy; and -dimethylaminoethoxyethoxy.

In one aspect, a nucleotide analogue or equivalent within the EON comprises one or more base modifications or substitutions. Modified bases comprise synthetic and natural bases such as inosine, xanthine, hypoxanthine and other-aza, deaza, -hydroxy, -halo, -thio, thiol, -alkyl, -alkenyl, -alkynyl, thioalkyl derivatives of pyrimidine and purine bases that are or will be known in the art. Purine nucleobases and/or pyrimidine nucleobases may be modified to alter their properties, for example by amination or deamination of the heterocyclic rings. The exact chemistries and formats may vary from oligonucleotide construct to oligonucleotide construct and from application to application, and may be worked out in accordance with the wishes and preferences of those of skill in the art.

An EON as disclosed herein is normally longer than 10 nucleotides, preferably more than 11, 12, 13, 14, 15, 16, still more preferably more than 17 nucleotides. In one aspect the EON as disclosed herein is longer than 20 nucleotides. The EON as disclosed herein is preferably shorter than 100 nucleotides, still more preferably shorter than 60 nucleotides, still more preferably shorter than 50 nucleotides. In a preferred aspect, the EON as disclosed herein comprises 18 to 70 nucleotides, more preferably comprises 18 to 60 nucleotides, and even more preferably comprises 18 to 50 nucleotides. Hence, in a particularly preferred aspect, the EON as disclosed herein comprises 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides. In one embodiment, the EON is 27, 28, 29, or 30 nucleotides in length.

In one aspect, at either end or both termini of an EON as disclosed herein, inverted deoxyT or dideoxyT nucleotides are incorporated.

### EXAMPLES

### Example 1. Editing of a Target Adenosine in a Human B4GALT1 Target RNA Molecule Using An In Vitro Biochemical Editing Assay

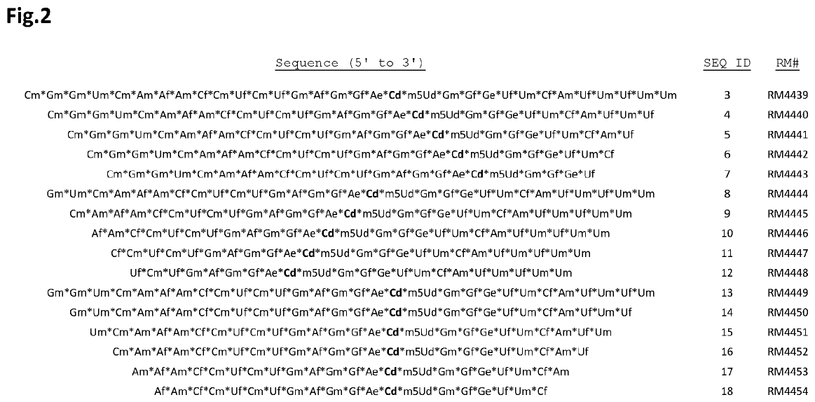

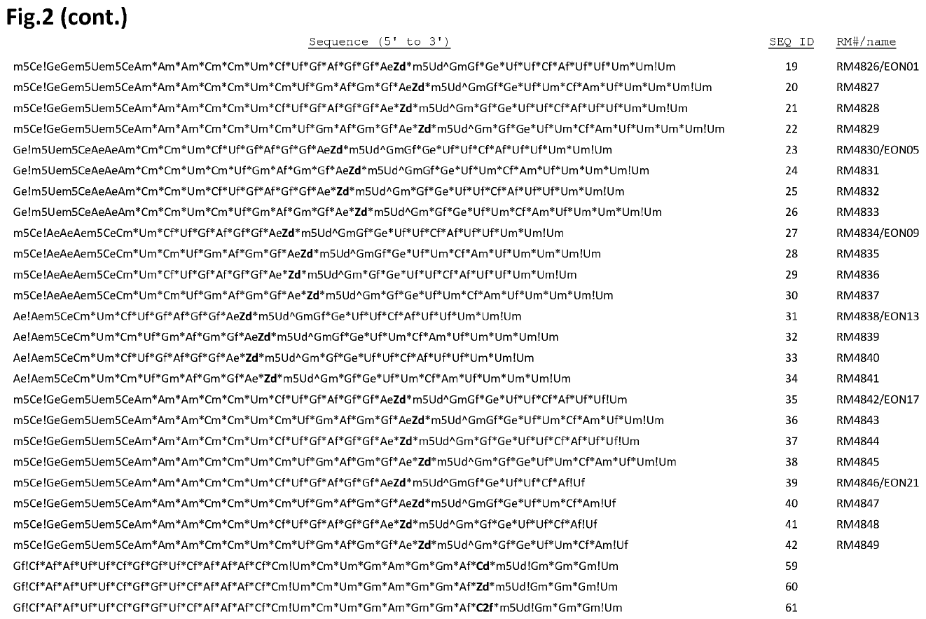

First, an initial set of the B4GALT1-targeting EONs (shown in FIG. 2) are tested to address editing of human B4GALT1 target (pre-) mRNA in an in vitro biochemical editing assay. To obtain the B4GALT1 target RNA a PCR is performed using a B4GALT1 G-block (IDT) which contains the sequence for the T7 promotor and (a part of) the sequence of HFE as template using forward primer 5′-CTC GAC GCA AGC CAT AAC AC-3′ (SEQ ID NO:43) and reverse primer 5′-TGG ACC GAC TGG AAA CGT AG-3′ (SEQ ID NO:44). The 5′ to 3′ G-block sequence (SEQ ID NO: 45) is as follows, in which the target adenosine is underlined and in bold, and in which the primer sequences are underlined:

TCTGGCTCGACGCAAGCCATAACACTAATACGACTCACTATAGGGTTAGTTTTTAGAGGCATGTCTATATCTCGCCC
AAATGCTGTGGTCGGGAGGTGTCGCATGATCCGCCACTCAAGAGACAAGAAAAATGAACCCAATCCTCAGAGGTTTG
ACCGAATTGCACACACAAAGGAGACAATGCTCTCTGATGGTTTGAACTCACTCACCTACCAGGTGCTGGATTACGTT
TCCAGTCGGTCCACGTTTG

The PCR product is then used as template for the in vitro transcription. The MEGAscript T7 transcription kit is used for this reaction. The RNA is purified on a urea gel and then extracted in 50 mM Tris-CI pH 7.4, 10 mM EDTA, 0.1% SDS, 0.3 M NaCl buffer and subsequently phenol-chloroform purified. The purified RNA is used as target in the biochemical editing assay.

Initially, EONs RM4439-RM4454 and RM4826-RM4849 are each annealed to the B4GALT1 target RNA, which is done in a buffer (5 mM Tris-CI pH 7.4, 0.5 mM EDTA and 10 mM NaCl) at the ratio 1:3 of target RNA to oligonucleotide (600 nM oligonucleotide and 200 nM target). The samples are heated at 95° C. for 3 min and then slowly cooled down to RT. Next, the editing reaction is carried out. The annealed oligonucleotide/target RNA is mixed with protease inhibitor (cOmplete™, Mini, EDTA-free Protease I, Sigma-Aldrich), RNase inhibitor (RNasin, Promega), poly A (Qiagen), tRNA (Invitrogen) and editing reaction buffer (15 mM Tris-CI pH 7.4, 1.5 mM EDTA, 3% glycerol, 60 mM KCl, 0.003% NP-40, 3 mM MgCl₂ and 0.5 mM DTT) such that their final concentration is 6 nM oligonucleotide and 2 nM target RNA. The reaction is started by adding purified ADAR2 (GenScript) to a final concentration of 6 nM into the mix and incubated for predetermined time points at 37° C. Each reaction is stopped by adding 95 μl of 95° C. 3 mM EDTA solution. A 6 μl aliquot of the stopped reaction mixture is then used as template for cDNA synthesis using Maxima reverse transcriptase kit (Thermo Fisher) with random hexamer primer (ThermoFisher Scientific). Initial denaturation of RNA is performed in the presence of the primer and dNTPs at 95° C. for 5 min, followed by slow cooling to 10° C., after which first strand synthesis is carried out according to the manufacturer's instructions in a total volume of 20 μl, using an extension temperature of 62° C. Products are amplified for pyrosequencing analysis by PCR, using the Amplitaq gold 360 DNA Polymerase kit (Applied Biosystems) according to the manufacturer's instructions, with 1 μl of the cDNA as template. Then PCR is performed using the following thermal cycling protocol: Initial denaturation at 95° C. for 5 min, followed by 40 cycles of 95° C. for 30 sec, 58° C. for 30 sec and 72° C. for 30 sec, and a final extension of 72° C. for 7 min.

Because inosines base-pair with cytidines during the cDNA synthesis in the reverse transcription reaction, the nucleotides incorporated in the edited positions during PCR will be guanosines. The percentage of guanosine (edited) versus adenosine (unedited) is defined by pyrosequencing. Pyrosequencing of the PCR products and data analysis is performed by the PyroMark Q48 Autoprep instrument (QIAGEN) following the manufacturer's instructions with 10 μl input of the PCR product and 4 μM sequencing primer: The analysis performed by the instrument provides the results for the selected nucleotide as a percentage of adenosine and guanosine detected in that position, and the extent of A-to-I editing at a chosen position is therefore measured by the percentage of guanosine in that position.

### Example 2. Editing of Human B4GALT1 Transcripts in HepG2 Cells

Editing of the endogenous human B4GALT1 transcript was investigated in human cells. For this, human HepG2 hepatocellular carcinoma cells were cultured in EMEM+10% FBS+1% P/S. Cells were kept at 37° C. in a 5% CO₂ atmosphere. In an initial experiment the EONs named RM4826 to RM4849 (see FIG. 2) were tested for editing efficiency. A total of 0.75×10⁵ HepG2 cells were seeded and treated with 1 or 5 μM EON, and 1 μM AG1856 (a saponin, also referred to as a triterpene glycoside; see WO2021/122998) was added per well. This mixture was kept on the cells for 72 hrs. Then, cells were collected, and total RNA was isolated from the transfected cells using the ReliaPrep™ RNA Miniprep kit. After removal of the culture medium, cells were washed once with PBS. After complete aspiration of the PBS, 100 μL lysis buffer was added to the wells to lyse the cells and collect the intracellular material. After addition of 35 μL isopropanol the mixtures were loaded in a column and subjected to several wash steps and DNase I treatment. After elution in a total volume of 15 μL DNase/RNase-free water, the RNA yield was determined using spectrophotometric analysis (NanoDrop) and stored at −80° C.

Maxima reverse transcriptase (RT, Thermo Fisher) was used to generate complementary DNA (cDNA). Typically, 500 ng total RNA was used in reaction mixture containing 4 μL 5×RT buffer, 2 μL dNTP mix (10 mM each), 0.5 μL Oligo (dT), 0.5 μL random hexamer and 0.5 μL Maxima reverse transcriptase (all Thermo Fisher) supplemented with DNase and RNase free water to a total volume of 20 μL. Samples were loaded in a T100 thermocycler (Bio-Rad) and initially incubated at 10 min at 25° C., followed by a cDNA reaction temperature of 30 min at 50° C. and a termination step of 5 min at 85° C. Samples were cooled down to 4° C. prior storing at −20° C.

To determine the editing efficiency, cDNA samples were used in two multiplex digital PCR (dPCR) assays. HepG2 cDNA samples were diluted 5 times before dPCR measurements. The first dPCR is designed to distinguish between cDNA species containing the original adenosine or the edited inosine, which is converted into a guanidine during cDNA synthesis. The first dPCR also quantifies the amount of B4GALT1 specific cDNA molecules in the mixture using a primer/probe set targeting exons 1 and 2. The second dPCR is designed to measure B4GALT1 exon 5 skip and housekeeping gene HPRT1. The primer and probe sequences are listed in Table 1.

TABLE 1
Primer and probe names and sequences (+ refers to an LNA nucleotide at the 3′ side)

| Name | Sequence 5′ to 3′ |
|---|---|
| hB4GALT1_e05_fw | TCCGCCACTCAAGAGACAAG (SEQ ID NO: 46) |
| hB4GALT1_e06_rv1 | GCACCTGGTAGGTGAGTGAG (SEQ ID NO: 47) |
| hB4GALT1_e01_fw | CCCGCTAGCAACTTGACCTC (SEQ ID NO: 48) |
| hB4GALT1_e02_rv | CCAGGTCCACAGGCATGTTA (SEQ ID NO: 49) |
| hB4GALT1_e04_fw | ATTATTGGGGCTGGGGAGGA (SEQ ID NO: 50) |
| hB4GALT1_e06_rv2 | AGCACCTGGTAGGTGAGTGA (SEQ ID NO: 51) |
| hHPRT1 e01-02 Fw 01 | CCTGGCGTCGTGATTAGTGA (SEQ ID NO: 52) |
| hHPRT1 e02-03 Rv 01 | CGAGCAAGACGTTCAGTCCT (SEQ ID NO: 53) |
| hB4GALT1_e05_edit_FAM | /56-FAM/GAACCC+A+G+TCCTCAG+AG/3IABkFQ/ |
| | (SEQ ID NO: 54) |
| hB4GALT1_e05_ori_HEX | /5HEX/A+TGA+ACCC+A+A+TCCTCAG+AG/3IABkFQ/ |
| | (SEQ ID NO: 55) |
| hB4GALT1_e01-02_TEX | /5TEX615/AGGAGTCCCCGCTGCTTGTG/3IAbRQSp/ |
| | (SEQ ID NO: 56) |
| hB4GALT1_e04-06_Cy5 | /5Cy5/GA+TGACAT+TTTTA+ACAGGTT+TG+ACC/3IAbRQSp/ |
| | (SEQ ID NO: 57) |
| hHPRT1_e02_TEX_01 | /5TEX615/GC+TGAGGATTTGGAAAGGGTGT (SEQ ID NO: 58) |

Digital PCR was performed using the QIAcuity 4, 5-plex, a QIAcuity PCR kit and 96-well 8.5K Nanoplates (Qiagen). In total 1.2 μL of the diluted cDNA mix was used in a dPCR mixture containing 3 μL 4×QIAcuity Mastermix, 0.6 μL per primer (10 UM stock concentration) and 0.3 μL per probe (10 μM stock concentration) supplemented with DNase and RNase free water to a total volume of 12 μL. The dPCR mixture was prepared in a pre-plate and then transferred into a 96-well 8.5 K Nanoplate and sealed with a Nanoplate seal. The plate was then transferred to the QIAcuity Four machine. First a priming and rolling step was performed to generate and isolate chamber partitions, followed by an amplification step using the following cycling protocol: 95° C. for 2 min for enzyme activation, 95° C. for 15 sec for denaturation, and 60° C. for 30 sec for annealing/extension for 40 cycles. The amplification step was followed by an image acquisition step of all wells. Data was analysed using the QIAcuity Suite Software (Qiagen).

The total amount of copies/ng RNA were determined by taking the sum of A-containing partitions, G-containing partitions, and exon 5 skip containing partitions per ng RNA. Percentage of A-to-I editing was determined by dividing the number of G-containing partitions by the total (G-plus A-containing partitions) per ng RNA multiplied by 100. Percentage of exon 5 skip was determined by dividing the number of exon 5 skip-containing partitions by the total copies/ng RNA.

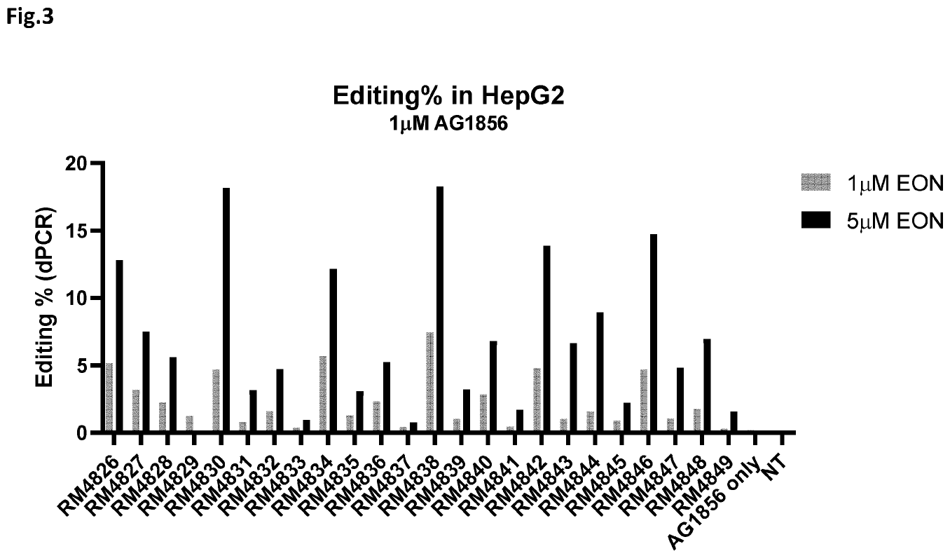

Results of the RNA editing of the endogenous B4GALT1 transcript are provided in FIG. 3, which shows that the efficiency varied significantly between EONs, but that an increase from 1 μm EON to 5 μM EON provided higher editing levels. No editing was observed in the negative controls (AG1856 only, and non-treated samples (NT)). RM4826, RM4830, RM4834, RM4838, RM4842, and RM4846 appeared to give the highest RNA editing levels.

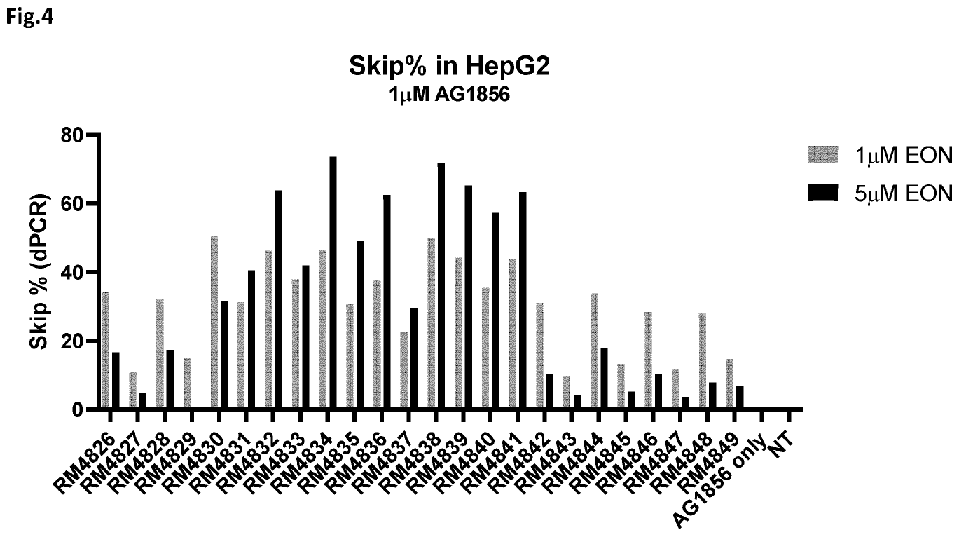

Since the binding of the EON to the target transcript may also interfere with splicing, and may or may not induce exon skipping events, it was investigated whether exon 5 of the B4GALT1 pre-mRNA was skipped during splicing. The c. 1055A target adenosine is in exon 5 of the B4GALT1 gene. The results are shown in FIG. 4 and indicate that some EONs gave a very high level of exon skipping (in some cases more than 70%), but the levels appear to be unrelated to the level of editing observed with the best 6 performing editing EONs mentioned above. The highest exon skipping percentages were observed with RM4832, RM4834, RM4836, RM4838, RM4839, RM4840, and RM4841. Low (er) levels of exon skipping were observed with RM4826, RM4827, RM4828, RM4829 (the 5 μM sample was lost in the process), RM4842, RM4843, RM4844, RM4845, RM4846, RM4847, RM4848, and RM4849. Interestingly, for the EONs that gave a lower percentage of skipping, these lower levels of exon skipping were observed in the samples with the higher concentration of EON (5 μM), instead of the lower concentration (1 μM). It remains to be determined whether exon skipping, induced by the treatment with the RNA editing oligonucleotides is an unwanted effect, or in contrast, a bonus effect, as outlined herein. The predominant reason to target c. 1055A in the B4GALT1 transcript is to lower the functionality of the B4GALT1 protein. Skipping exon 5 of the transcript (which is an in-frame event) may also result in a (shorter) protein with a lowered activity, which may be an additional beneficiary effect of the EON treatment.

### Example 3. Editing of Human B4GALT1 Transcripts in Liver Spheroids

Next, it was investigated whether RNA editing could also be achieved on endogenous B4GALT1 transcripts in liver spheroids grown from primary human hepatocytes. To generate spheroids, female primary human hepatocytes (PHH) cells (BioIVT) were used. As per the supplier's protocol, 1,500 cells/well of PHH cell suspension (15,000 cells/mL) were plated in a Nuclon Sphera low attachment U-bottom 96 well plates using INVITROGRO Spheroid Plating Medium in combination with Spheroid Medium Supplement A, TORPEDO Antibiotic Mix, and INVITROGRO Spheroid Spin Medium (all from BioIVT). The plates were then incubated for 5 days at 37° C. in a 5% CO₂ atmosphere.

After the incubation, the spheroids were transferred and pooled into Flat-bottom 96 well plates. The pooling resulted in 8 spheroids per well, with a total medium volume of 100 μL/well. To maintain the spheroid cultures, 100 μL of maintenance medium was added to each well and incubated for another 48 hrs at 37° C. in a 5% CO₂ atmosphere. The maintenance medium consisted out of INVITROGRO Spheroid Maintenance Medium (BioIVT), combined with Spheroid Medium Supplement A, which was also used during the treatment of the spheroids. Then, 100 μL of medium was removed from each well (containing the 7-day-old spheroids) and 100 μL of the treatment condition was added. Each well was treated with 1 or 5 μM EON and 1 μM AG1856 saponin (see above) for 72 hrs. For this initial spheroid experiment, four of the best performing EONs in cells (see above) were selected: RM4834, RM4838, RM4842, and RM4846. Negative controls were saponin only and NT samples.

After EON/saponin exposure, spheroids were collected, washed once with PBS and then 300 μL lysis buffer was added. RNA isolation, RNA yield determination, cDNA generation, editing efficiency using dPCR and exon 5 skipping effect assessments were performed as described in Example 2.

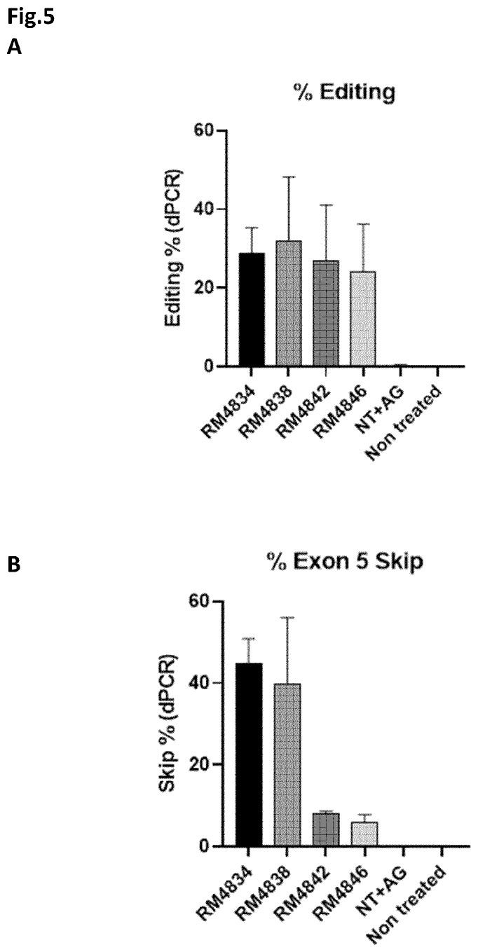

Results are shown in FIG. 5. In line with the results observed in the HepG2 cells, all four EONs could mediate relatively high levels of RNA editing of the endogenous B4GALT1 transcript in the PHH grown spheroids, with levels up to more than 30% (FIG. 5A). All four EONs performed in a similar range. Like what has been shown in Example 2, also exon 5 skipping was assessed in these spheroids, and the results are given in FIG. 5B. Interestingly, treatment with RM4834 and RM4838 resulted in relatively high levels of exon 5 skip (more than 40%), whereas RM4842 and RM4846 only caused a skipping percentage around 8%. No editing or exon skipping could be determined in the samples treated with saponin only (NT+AG in FIG. 5) alone or in the non-treated samples. This shows that RNA editing to significant levels could be achieved with a variety of antisense oligonucleotides, which unexpectedly differed significantly in their ability to influence splicing of the human B4GALT1 pre-mRNA.

### Example 4. Effect of Fibrinogen Levels after Editing of Human B4GALT1 Transcripts In Vitro

To study the effect of RNA editing of the c. 1055A target adenosine in human B4GALT1 transcripts on the secretion of fibrinogen, it is tested whether such RNA editing gives lower levels of fibrinogen in the supernatant of human hepatocellular carcinoma cell lines HepG2 and Huh-7 cells after treatment with EONs. For this, HepG2 cells are cultured in EMEM+10% FBS+1% P/S and Huh-7 cells are cultured in RPMI+10% FBS+1% P/S. Cells are kept at 37° C. in a 5% CO₂ atmosphere. A total of 0.75×10⁵ HepG2 cells and separately 0.5×10⁵ Huh-7 cells are seeded in 24-wells plates and treated with 5 μM EON and 1 μM AG1856 (see above) per well for 72 hrs. Then, cell culture medium supernatant is collected. These samples are centrifuged at 2000 g for 10 min. Subsequently, the supernatant is transferred to a new tube. These samples are diluted 1:500 and the fibrinogen levels are measured using a high sensitivity fibrinogen ELISA (ab241383, Abcam), as follows. 50 μL sample or standard is added to each well. Subsequently, 50 μL of antibody cocktail is added to the wells. After 1 hr of incubation at RT, the wells are washed 3 times with wash buffer. Then, 100 μL of TMB Development Solution is added to each well and the plate is incubated for 10 min in the dark. Lastly, 100 μL of Stop Solution is added to each well. The plate is measured using a plate reader (SpectraMax M5) at 450 nm. The standard curve is generated using the 4PL method in GraphPad (version 9.0.1). Raw values are corrected for the blank and the average is calculated from duplicate measurements. The fibrinogen levels are determined via interpolation using the standard curve. The fibrinogen levels are normalized to the RNA yield of NT+medium condition.

### Example 5. Editing of Human B4GALT1 Transcripts in Primary Human Hepatocytes

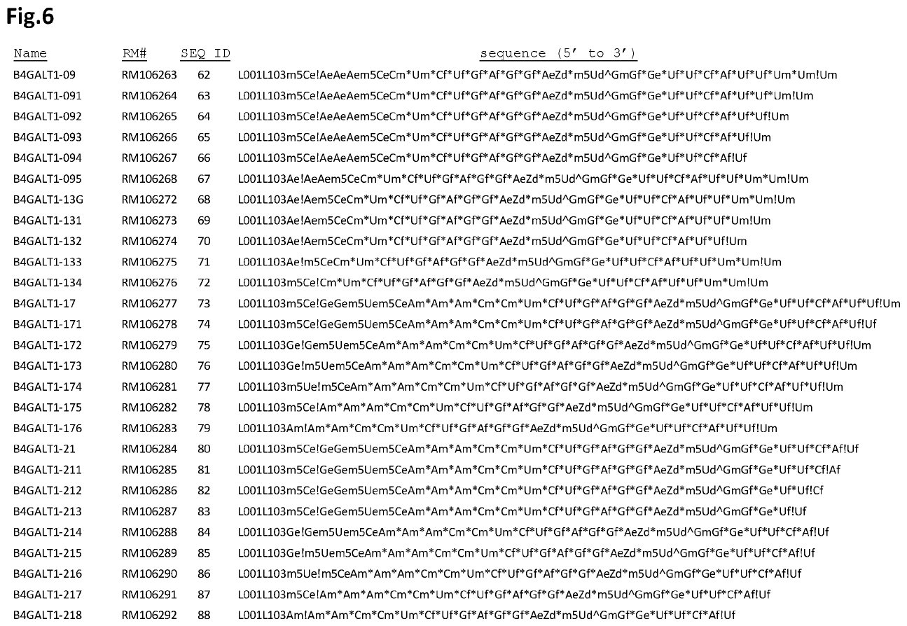

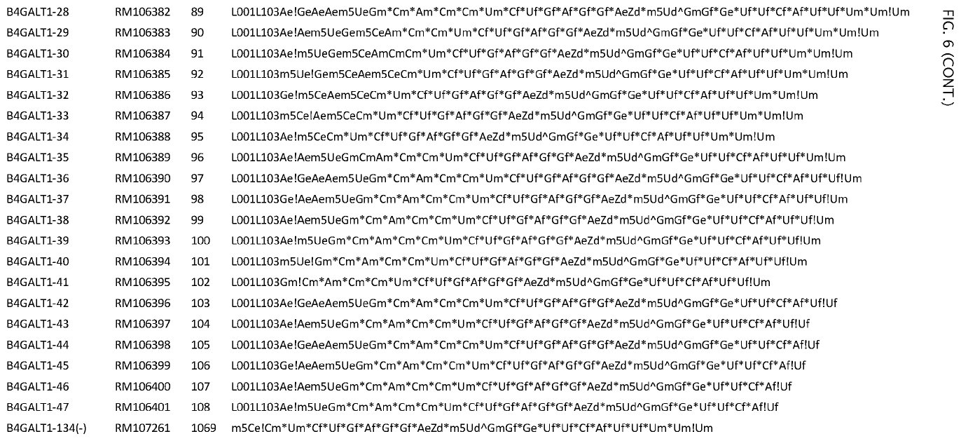

Next, a new set of EONs was designed, based on the best performing editors from the first editing screens (EON01 (RM4826), EON05 (RM4830), EON09 (RM4834), EON13 (RM4838), EON17 (RM4842), and EON21 (RM4846)). This new set of EONs with their respective chemical modifications is given in FIG. 6. Some of these EONs differ in the 5′ terminal part, since some EONs are complementary to exon 6 (hence, after splicing of intron 5 from the pre-mRNA), whereas some are complementary on the 5′ terminal part with intron 5 (hence, before splicing), see for instance the difference in the 5′ terminal parts of B4GALT1-32 (RM106386) and B4GALT1-218 (RM106292). Each one of the EONs in FIG. 6 (except for B4GALT1-134 (−)) comprises a tri-antennary GalNAc modification (L001=OP-042; Hongene Biotech) on the 5′ terminus, linked to the most terminal 5′ nucleotide through a TEG linker (L103) to stimulate entry into liver cells. The EONs and their attachments were manufactured according to standard protocols known to the person skilled in the art.

Using the new set of EONs, together with EON01 (RM4826) and EON05 (RM4830), editing of the endogenous human B4GALT1 transcript was investigated in primary human hepatocytes (PHH). A non-treated (NT) sample was taken along as a negative control. For this, a total of 0.5×10⁵ PHH cells (BioIVT) were plated using INVITGRO CP medium supplemented with TORPEDO Antibiotic Mix. Cells were kept at 37° C. in a 5% CO₂ atmosphere. Four hours after plating, the medium was refreshed with cultured in INVITROGRO HI medium supplemented with TORPEDO Antibiotic mix. The next day, cells were treated with 5 μM EON+1 UM AG1856 per well. This mixture was kept on the cells for 72 hrs. Then, the cells were washed once with PBS and 100 μL lysis buffer was added. Total RNA was isolated using the Direct-zol™ RNA Microprep kit (Zymo Research). After the addition of 100 UL ethanol (95-100%), the mixtures were loaded onto a column and subjected to several wash steps and DNase I treatment. After elution in a total volume of 15 μL DNase/RNase-free water, the RNA yield was determined using spectrophotometric analysis (NanoDrop) and stored at −80° C. Subsequently, RT reactions and dPCRs were performed as outlined in Example 2, with the indicated primers and probes.

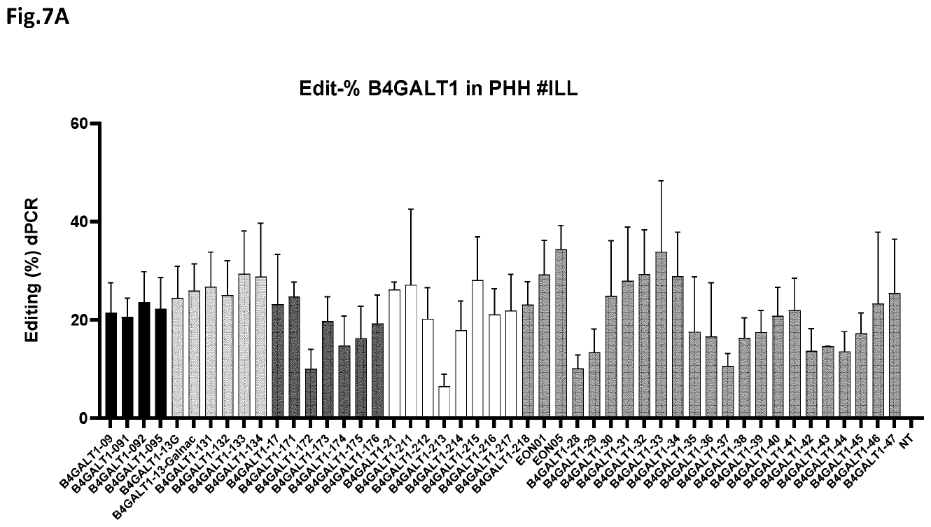

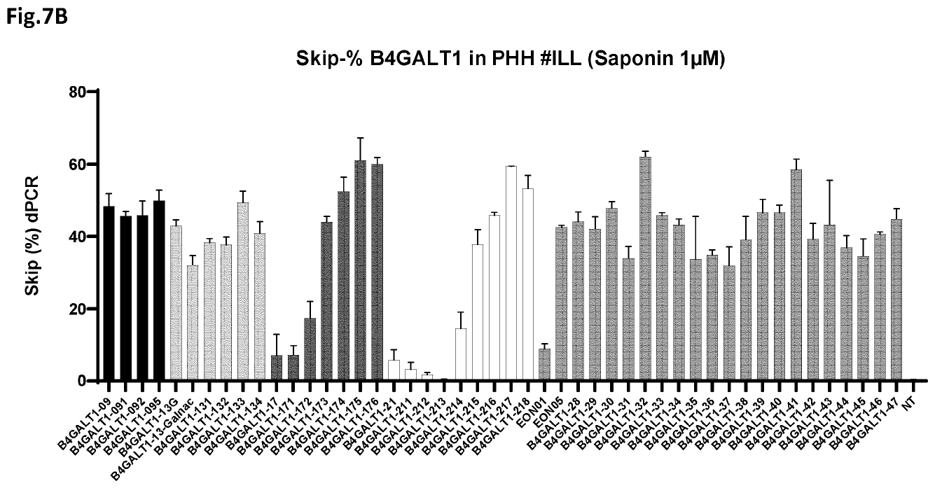

Results are shown in FIG. 7, wherein in (A) the percentage editing is depicted obtained in these PHHs, clearly showing that EON05 (without a GalNAc moiety) was still one of the best performing EONs. This can be explained by the possibility that the GalNAc interaction with its respective receptor is less important to boost entry in vitro, whereas it may add significantly in in vivo settings. In any case, some of the new EONs clearly showed a beneficial effect over some of the others, with for instance B4GALT1-33 as one of the best performers. FIG. 7(B) shows the percentage skip of exon 5 in the same samples, clearly demonstrating a strong difference between some EON treatments. For instance, exon skip was close to zero after incubation with B4GALT1-212 and -213, whereas B4GALT1-175 and -176 EONs caused a relatively high percentage of skip. Strikingly, while EON01 and EON05 performed similarly in editing, there was a strong difference in causing exon 5 skip between these two (non-GalNAc containing) EONs.

Then, in a next experiment, it was investigated whether RNA editing could also be achieved in PHHs without adding the saponin, hence by using a simple co-incubation of the oligonucleotides in the medium of the cells (=gymnotic uptake, or ‘gymnosis’). The entire experimental setup was identical to the above setup with the exception that no saponin was added together with the EONs. All downstream RNA purification and dPCR steps were as mentioned above.

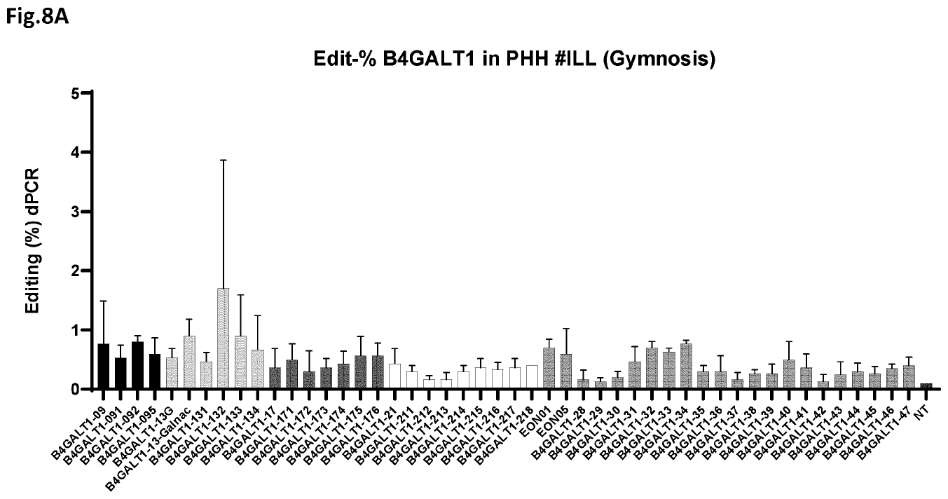

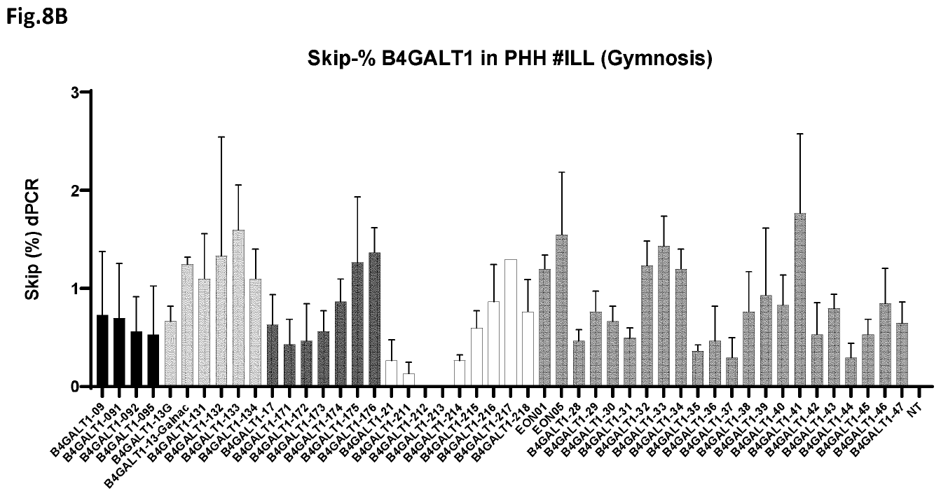

The results of this experiment are shown in FIG. 8, wherein (A) shows the percentage RNA editing after gymnotic uptake of the indicated EONs, which is clearly (and expectedly) lower than in FIG. 7 (where saponin was used as a ‘transfection agent’), but still significantly over background. It should be noted that low levels of editing do not necessarily mean that a downstream effect on the protein and the functionality thereof is also low, as described herein above. Editing percentages caused by the EONs was in the range of 1%, except for B4GALT1-132 that showed levels as high as 4%. The percentage of exon skipping (exon 5) was also determined in these samples. In line with the lower editing levels, also lower levels of exon skipping was found, see FIG. 8(B), with a similar pattern as was found in the experiment wherein saponin was used for improved entry of the oligonucleotides into the cells.

### Example 6. Editing of B4GALT1 Transcripts in Primary Mouse Hepatocytes

A similar experiment as outlined in example 5, in which no saponin was applied, was performed using primary mouse hepatocytes (PMH). Primary mouse hepatocytes were isolated from mouse liver using the Liver perfusion kit (Miltenyi) and the GentleMACS Octo Dissociator with Heaters (Miltenyi) according to manufacturer's instruction. In more detail, livers were dissected from mice, washed with PBS, and transferred to the GentleMACS Octo Dissociator with Heaters. An automated program 37C_m_LIPK_1 was run, consisting of different steps including priming, initial perfusion, washing, equilibration and enzymatic perfusion. After the perfusion process was completed, the perfused liver lobe and used enzyme solution were transferred to a gentIMACS C tube (Miltenyi). Subsequently, the gentIMACS C tube was transferred back into the Octo Dissociator and the program LIPK_HR_1 was run to further release hepatocytes from the perfused liver. Finally, the cell solution was passed through a MACS 100 μM SmartStrainer (Miltenyi) to enrich hepatocytes. A debris removal kit (Miltenyi) was used to remove debris from the cell solution. The cell pellet was resuspended in 5 mL DMEM+5% FBS+1% P/S. Cells were counted and a total of 0.75×10⁵ cells were seeded. Cells were treated with 5 μM EON using Williams E medium+2 mM Glutamine+1% P/S and this mixture was kept on the cells for 72 hrs. RNA isolation, RNA yield determination, cDNA generation, editing efficiency using dPCR and exon 5 skipping effect assessments were performed as described in Example 2. However, only one dPCR mix was used that combines assays for editing, exon 5 skip and B4GALT1 expression in exon 1-2 using primers and probes specifically for mouse, as given in Table 2.

TABLE 1
Primer and probe names and sequences (+ refers to an LNA nucleotide at the 3′ side)

| Name | Sequence 5′ to 3′ |
|---|---|
| mB4GALT1_e04_fw | ATTATTGGGGTTGGGGAGGA (SEQ ID NO: 1070) |
| mB4GALT1_e06_rv1 | CCAACACCTTGTAGGTAAGTGA (SEQ ID NO: 1071) |
| mB4GALT1_e05_edit_FAM | /56-FAM/TGAGCCC+A+G+TCCTCAG+AG/3IABkFQ/ |
| | (SEQ ID NO: 1072) |
| mB4GALT1_e05_ori_HEX | /5HEX/A+TG+AGCCC+A+A+TCCTCAG+AG/3IABkFQ/ |
| | (SEQ ID NO: 1073) |
| mB4GALT1_e01_fw | GGCTGTATTCCCCTCCA (SEQ ID NO: 1074) |
| mB4GALT1_e02_rv | GTTCAATGGGGTACTTCAGG (SEQ ID NO: 1075) |
| mB4GALT1_e01_e02_Ref_TEX | /5TEX615/AGGAGTCCCCGCTGCTCGTTG/3IAbRQSp/ |
| | (SEQ ID NO: 1076) |
| mB4GALT1_e04-06_Cy5 | /5Cy5/ACAT+TTT+TAA+CAGGT+TTGACCGGA+T/3IAbRQSp/ |
| | (SEQ ID NO: 1077) |

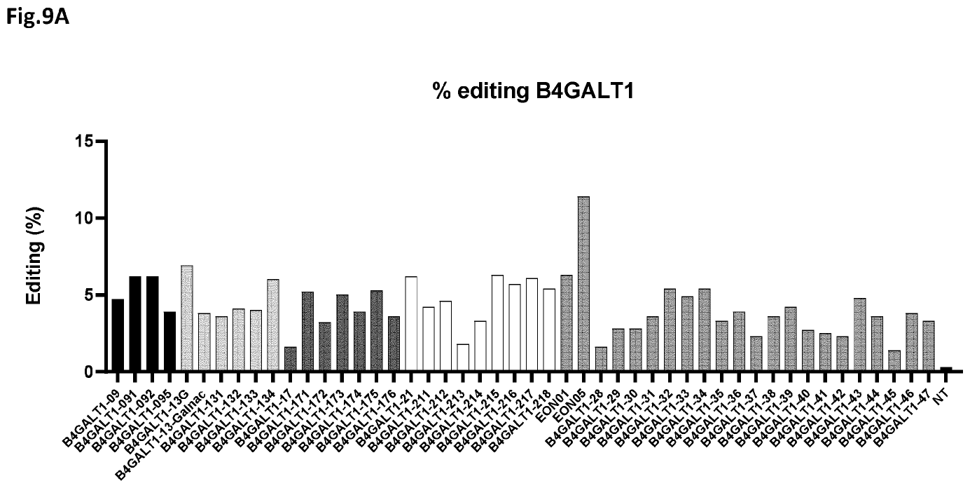

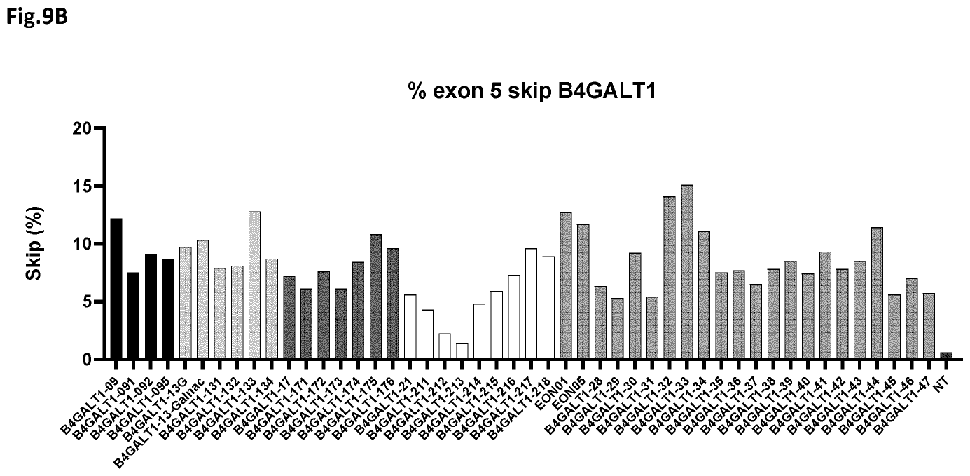

The results of these experiments are shown in FIG. 9, wherein (A) displays the percentage editing using the same EONs. These EONs are—except for the orphan nucleotide opposite the target adenosine—100% complementary to the target sequence in the human B4GALT1 (pre-) mRNA transcript. The target sequence in Mus musculus comprises a G five nucleotides from the target A in the 5′ direction, which means that the nucleotide at position-5 in the EONs of FIG. 6 (generally a uridine) forms a G: U base pair with the mouse target transcript sequence. As was shown in WO2017/220751, this is not necessarily unfavourable. Additional mismatches, bulges and/or wobbles on top of the mismatch at the orphan position may contribute to a better recognition of the ADAR enzymes in the cell of the double-stranded RNA complex that is formed and seen as a more suitable target configuration. If this is indeed the case, the data shown in FIG. 9(A) supports this, because higher levels of editing were observed in these PMH's than in PHH's (see FIG. 8(A)) under these circumstances (no saponin added). EON05 (RM4830; SEQ ID NO:23) performed best with editing levels reaching more than 10%. In line with the results in the PHHs, it remains to be seen how much the GalNAc moiety contributes to cell entry in in vitro setups like this. Nevertheless, RNA editing levels of the best performing EONs were in the range of 5%. Exon 5 skipping percentages, shown in FIG. 9(B), were along a similar pattern as observed with PHHs, described above.

### Example 7. Editing of Human B4GALT1 Transcripts in Primary Human Hepatocytes Using EONs Captured in Lipid Nanoparticles as a Delivery Vehicle

In a next experiment, it was investigated whether a different type of delivery method, in which the EONs are encompassed within a so-called Lipid Nanoparticle (LNP) would contribute to the delivery of the oligonucleotides to the target cells. For this, EON05 (RM4830; SEQ ID NO: 23; see FIG. 2), EON13 (RM4838; SEQ ID NO:31; see FIG. 2), and B4GALT1-134 (−) (RM107261; SEQ ID NO: 1069), which is identical to B4GALT1-134 but without a GalNAc moiety and TEG linker, and herein also referred to as EON134; see FIG. 6) were selected.

LNP formulations containing the EONs were manufactured as disclosed in WO2015/048020. Lipid stock was first prepared in ethanol. Then, these lipids were mixed in a molar ratio of DLin-MC3-DMA (50), Cholesterol (38.5), DSPCV (10) and PEG-200-DMG (1.5). The formulation process can be summarized as follows. The respective EONs were mixed with lipids in a ratio of 1 to 3.6 in a microfluidic device. Formulated LNPs were further concentrated in Tangential Flow Filtration (TFF) system, undergoing 12 hr dialysis for buffer exchange, and then sterilized by sterile filtration. The final solution concentration was 1 mg/mL. Storage of the LNP formulations containing EONs was in glass vials at 2-8° C. until further use.

PHHs were obtained and plated as described in Example 5. A total of 0.5×10⁵ cells per well were seeded 24 hr before incubation with the LNP formulations. A concentration range of 0.01 to 10 UM EON05-LNP, EON13-LNP, and EON134-LNP in INVITROGRO HI medium+TORPEDO Antibiotic mix+10% FBS was used in per well for 72 hr. Harvesting, RNA isolation, cDNA preparation, and dPCR procedures were performed as described in Example 2.

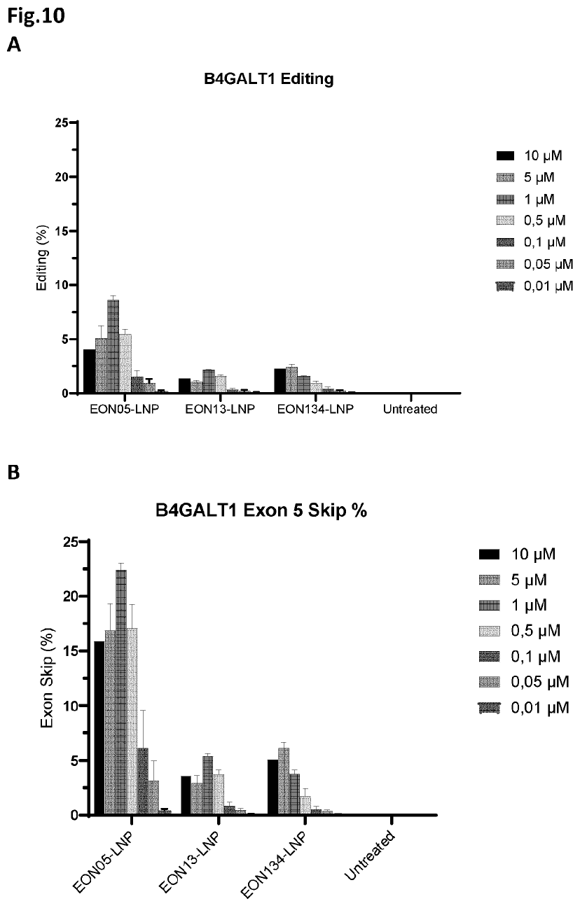

FIG. 10(A) shows the editing percentages that were obtained in the PHHs using the three LNP formulations as indicated using the different indicated concentrations and using an untreated sample as a negative control. This shows that at the time point that the cells were harvested, a concentration of 1 μM worked best under these conditions, and that (without the aid of saponin) editing levels up to 10% could be reached by using the EON05-LNP formulation. Like the experiments described above, the percentage exon 5 skip was also determined in these samples, which gave the predicted results based on the percentage editing that was determined, see FIG. 10(B).

### Example 8. Editing of B4GALT1 Transcripts in Mouse Liver Cells

The three LNP preparations described in Example 7 (EON05-LNP, EON13-LNP, and B4GALT1-134(−) (~EON134-LNP)) were also used in an in vivo experiment in which mice were injected intravenously (IV) with the LNP formulations or controls. The setup of the in vivo experiment was as follows, in which the dose level of the oligonucleotide was 3 mg/kg body weight for all mice, except that the repeated dosing in group 6 and the initial dosing of the mice for day 2 and day 4 in group 5 was 1.5 mg/kg body weight:

| | | | Necropsy | Group | |
|---|---|---|---|---|---|
| Group | Test particle | Dosing days | days | size | Total |
| 1 | vehicle | 0 | 2, 4, 7 | 8 | 24 |
| 2 | vehicle | 0, 7, 14, 21, 28 | 30 | 8 | 8 |
| 3 | Actb-LNP | 0 | 2, 4, 7 | 6 | 18 |
| 4 | B4GALT1-05-LNP | 0 | 2, 4, 7 | 6 | 18 |
| 5 | B4GALT1-13-LNP | 0 | 2, 4, 7 | 6 | 18 |
| 6 | B4GALT1-13-LNP | 0, 7, 14, 21, 28 | 30 | 8 | 8 |
| 7 | B4GALT1-134-LNP | 0 | 2, 4, 7 | 6 | 18 |

The negative control vehicle was an LNP formulation without any oligonucleotide, whereas the Actb-LNP was an LNP formulation with an oligonucleotide (RM3891) against a human Actin B target sequence, which formulation also served as a negative control in this experiment.

At the indicated necropsy days, the mice were sacrificed according to standard procedures and then blood samples were collected and organs, including the liver, were dissected. RNA was isolated from liver tissue. 1 mL of Trizol (Thermo Fisher) was added to the liver tissue in 2 mL tubes with 1.4 mm ceramic beads (Thermo Fisher) and samples were homogenized using a Beadmill 24 for 25 sec at 6 m/s. Tissue homogenate was transferred to a 1.5 mL Eppendorf tube and 200 μL chloroform (VWR) was added per 1 mL homogenate. After centrifuging at 12.000 g for 15 min at 4° C., 300 μL of the aqueous layer was transferred to a new 1.5 mL Eppendorf tube. Then 300 μL isopropanol was added and the RNA isolation was proceeded using the ReliaPrep™ RNA Miniprep kit. The mixtures were loaded in a column and subjected to several wash steps and DNase I treatment. Elution was done using total volume of 50 μL DNase/RNase-free water. RNA yield determination, cDNA generation, editing efficiency using dPCR and exon 5 skipping effect assessments were performed as described in Example 2.

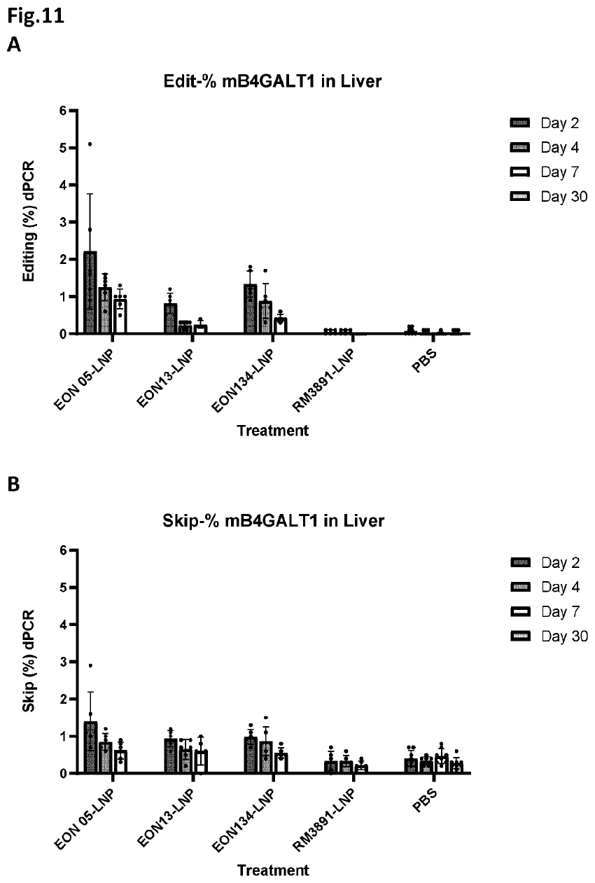

FIG. 11(A) shows the editing percentage in the liver of the mice in each of the treated groups at day 2, 4, 7, and 30, as indicated, in comparison to the Actb-LNP control (RM3891-LNP) and PBS. These results show that it was possible to reach editing levels as high as 2% two days after administration, which was significantly higher than what was observed in the livers of mice that were treated with the negative control. The editing percentages reduce over time, as seen in the samples of day 4, day 7 and day 30. FIG. 11(B) shows the percentage of exon 5 skip in the same samples, again confirming the results observed in the cells and liver spheroids above.

### Example 9. Editing of Human B4GALT1 Transcripts in Primary Human Hepatocytes

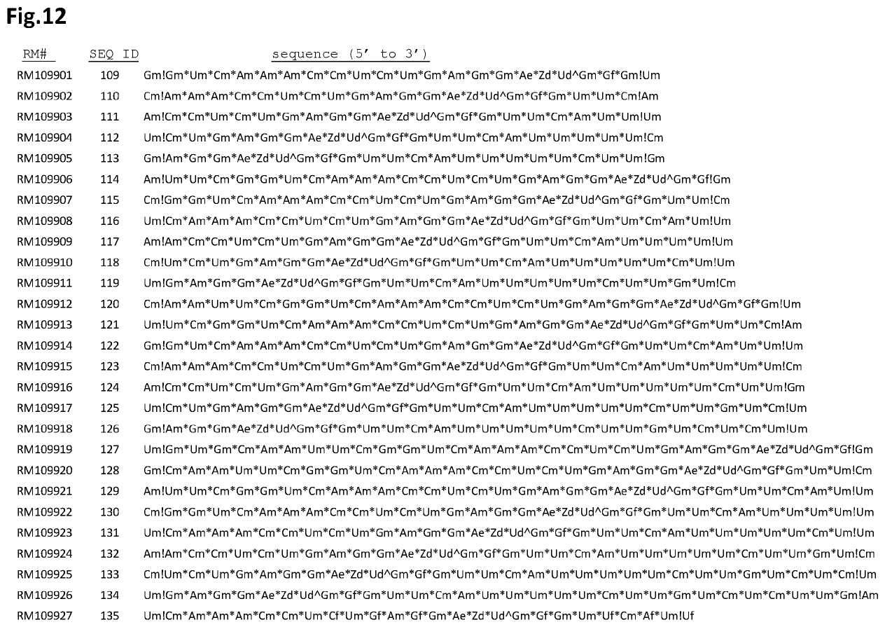

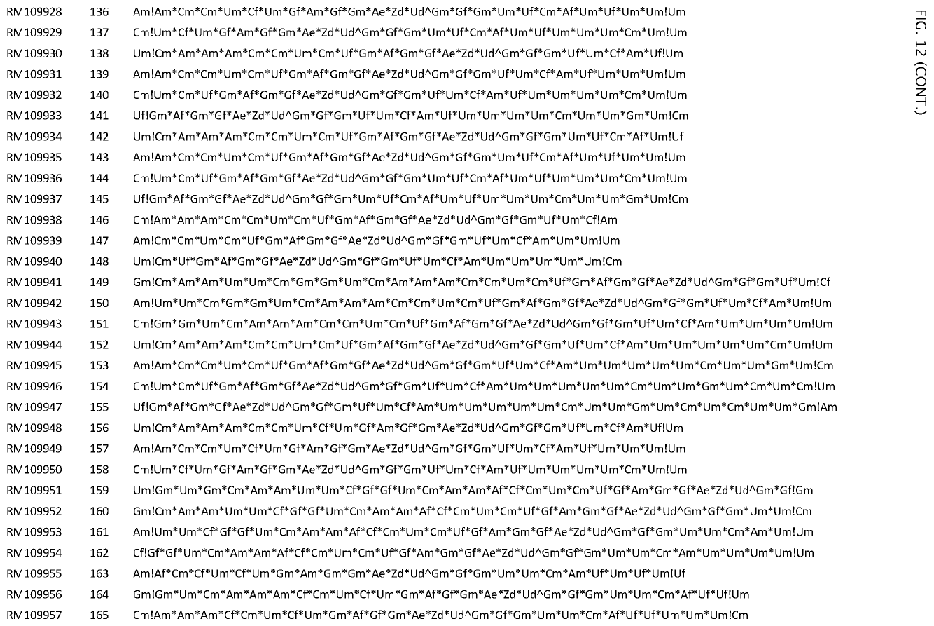

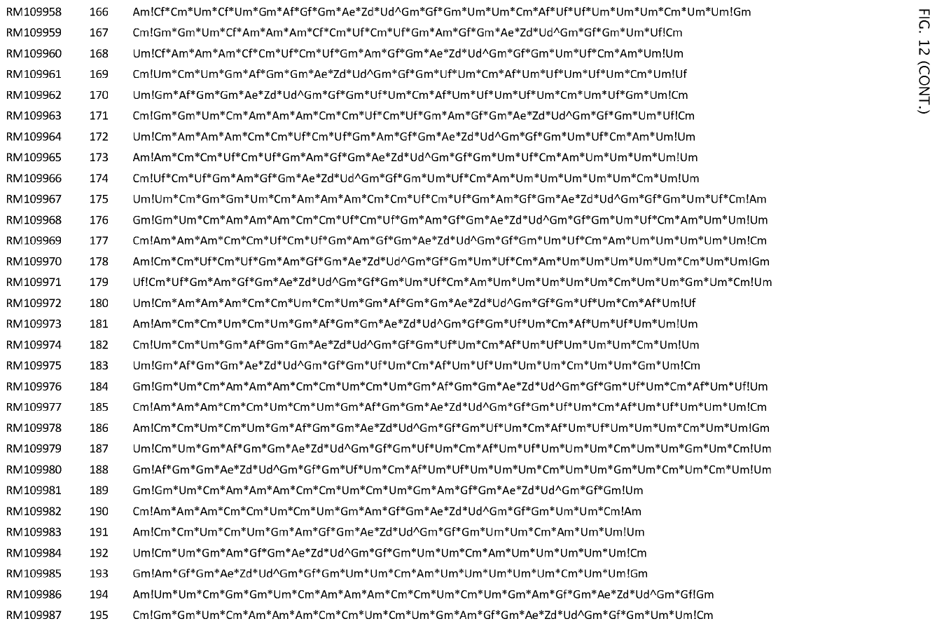

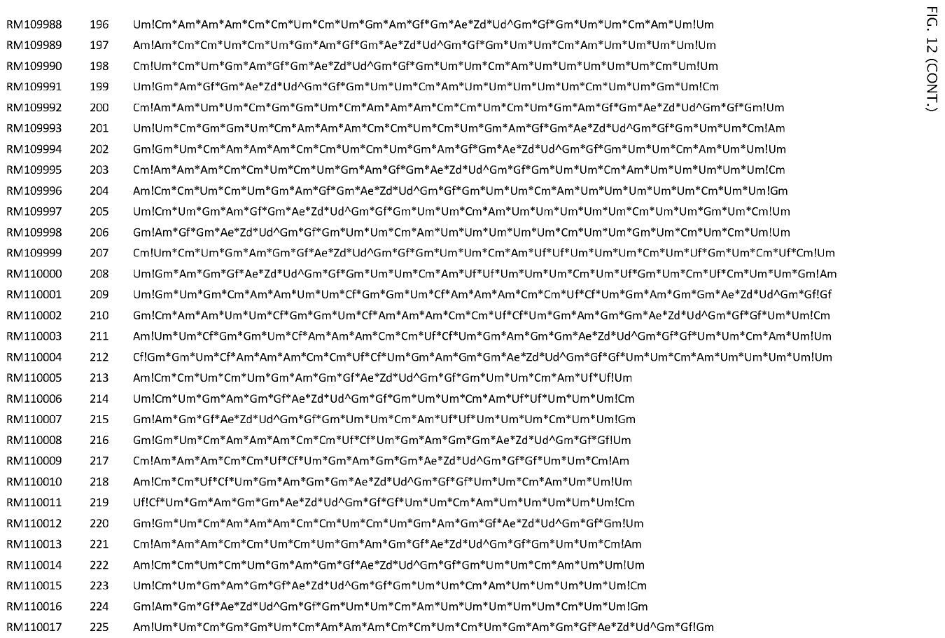

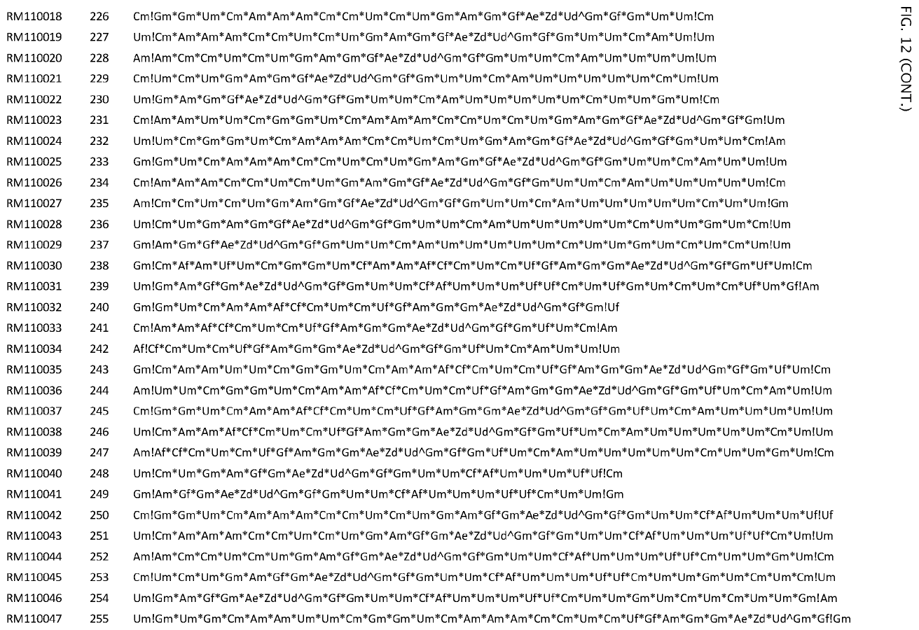

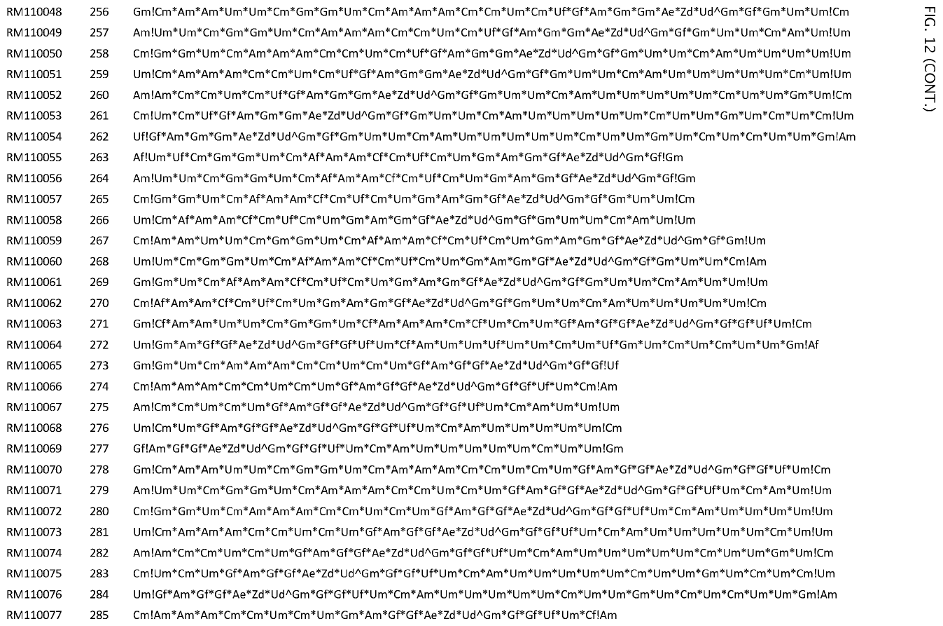

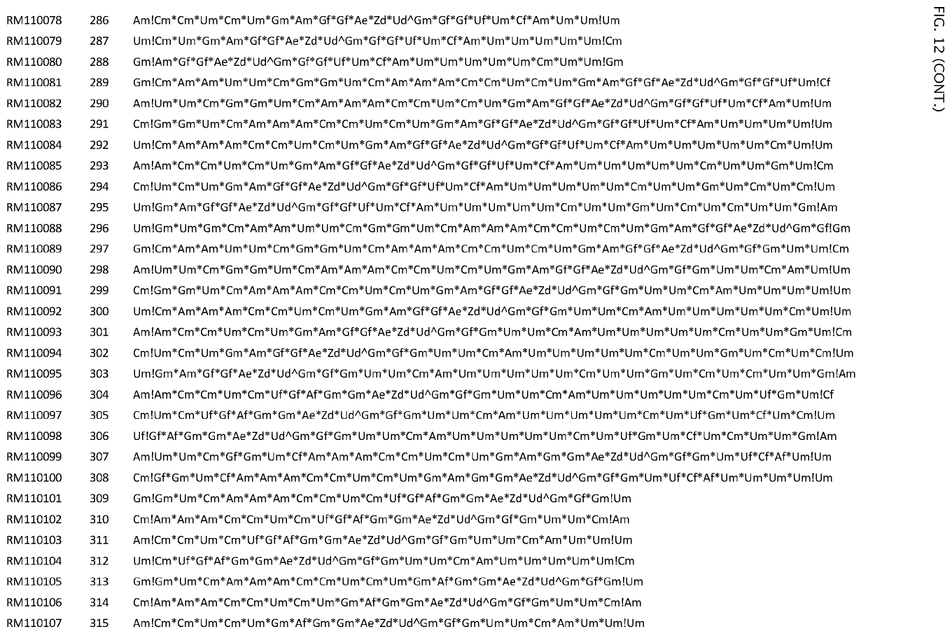

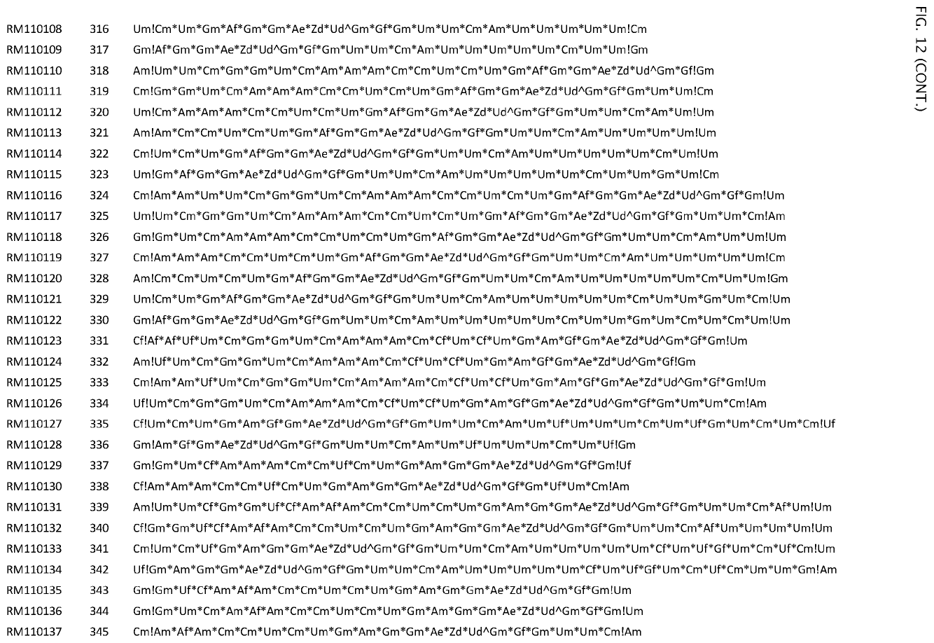

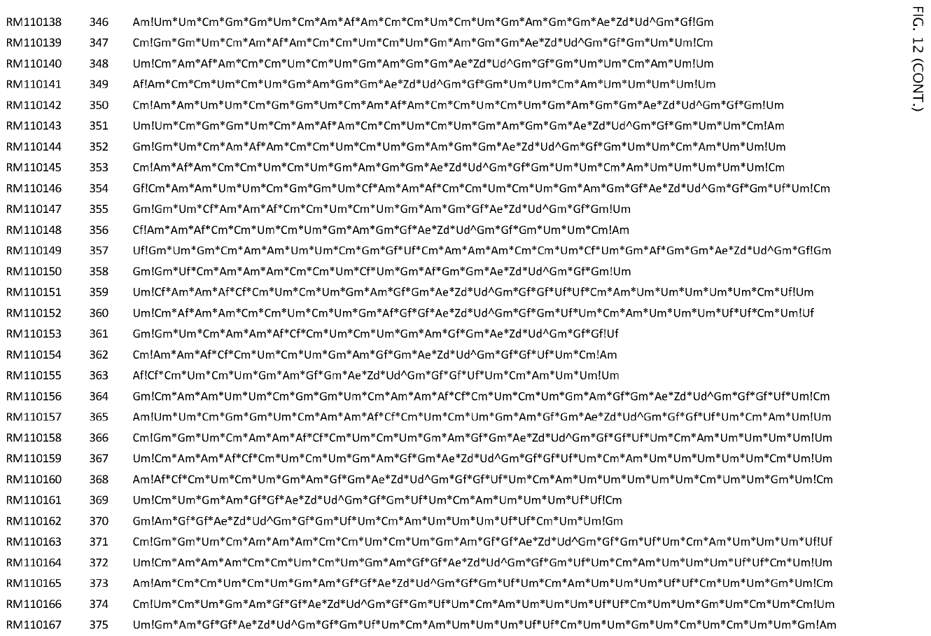

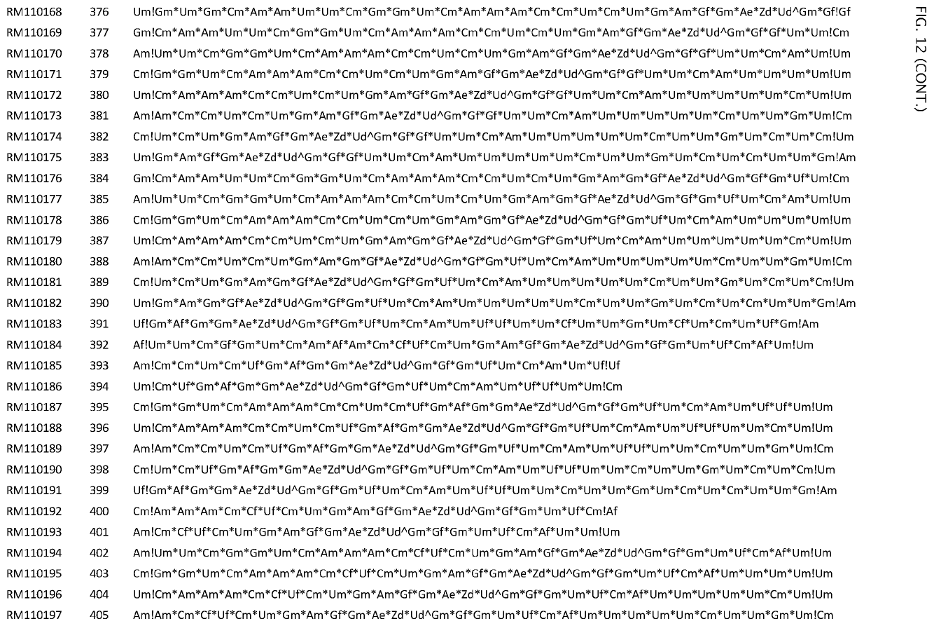

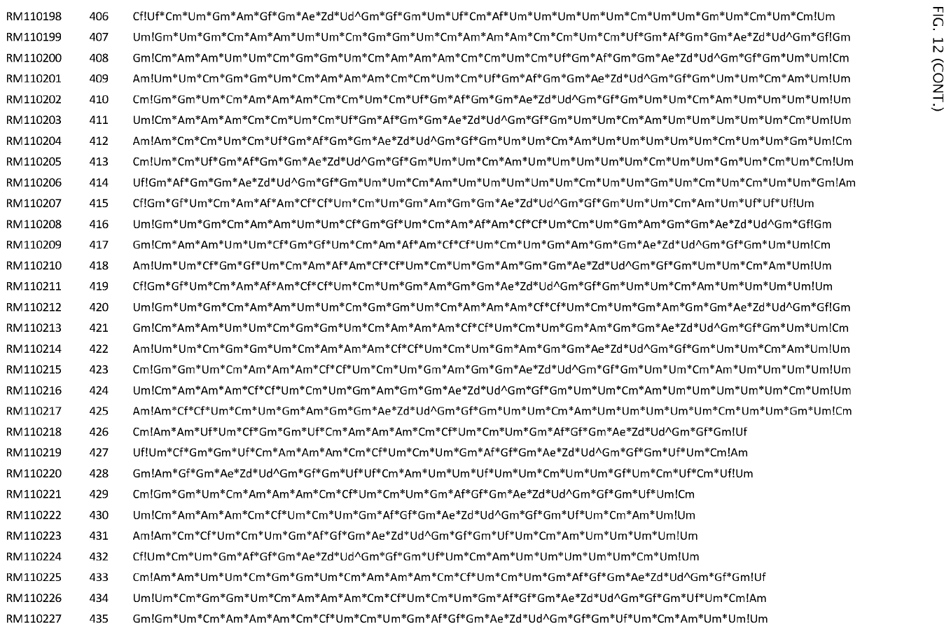

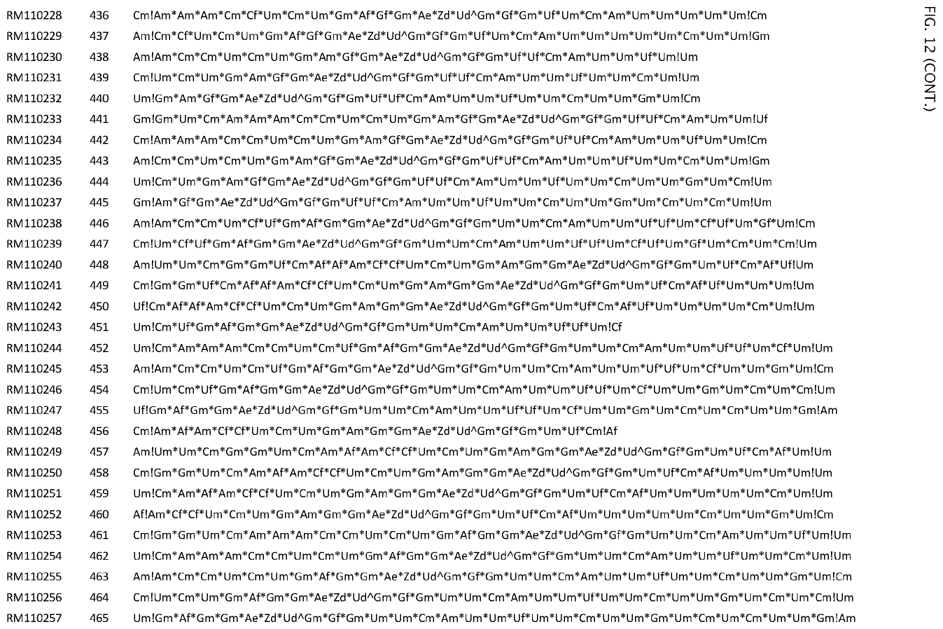

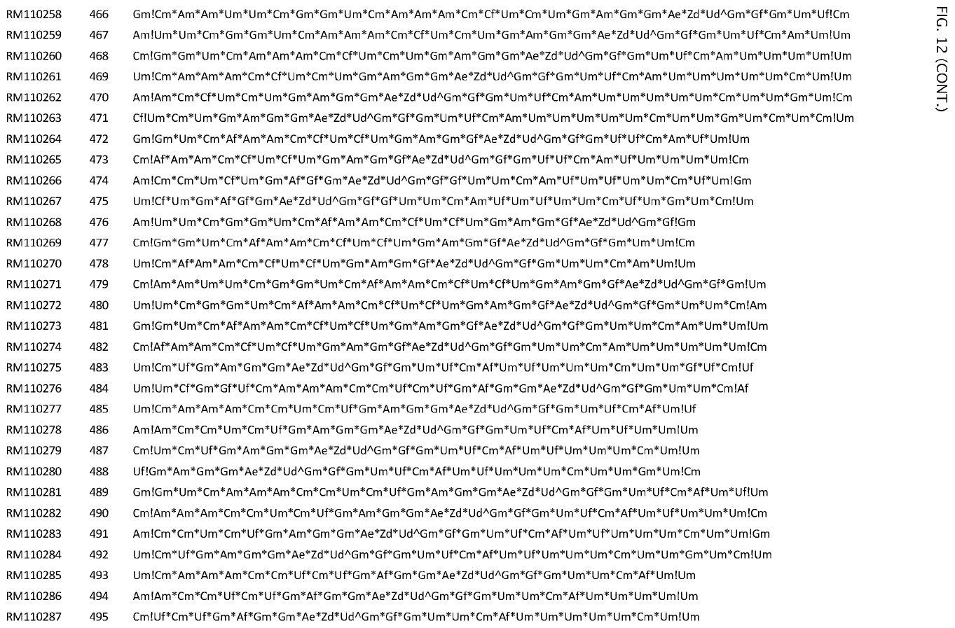

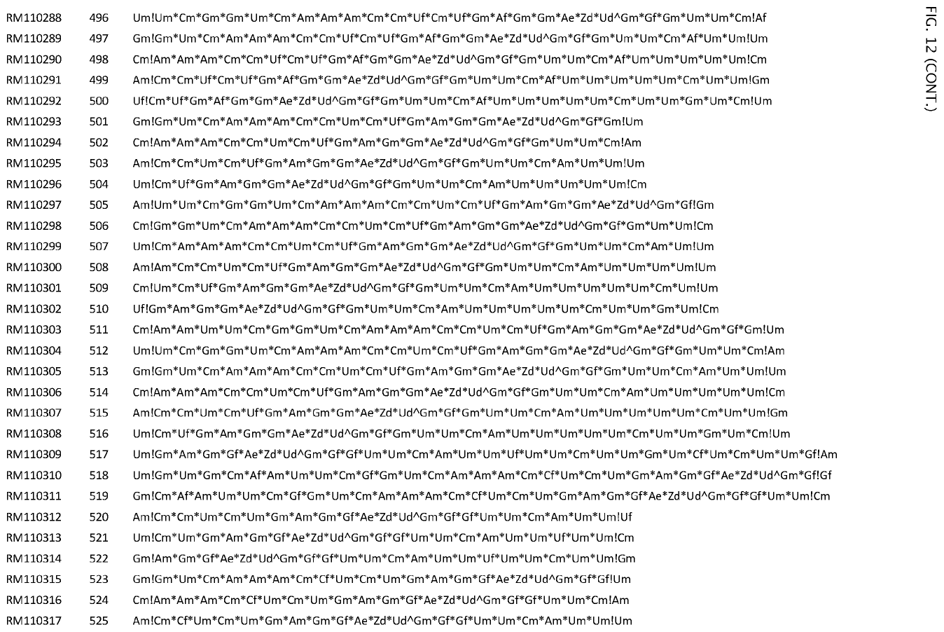

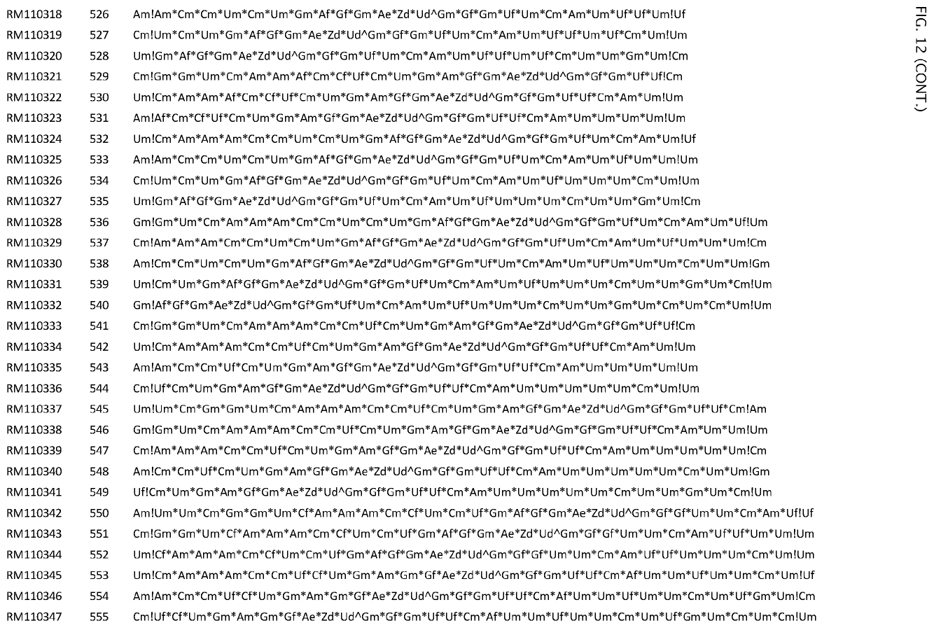

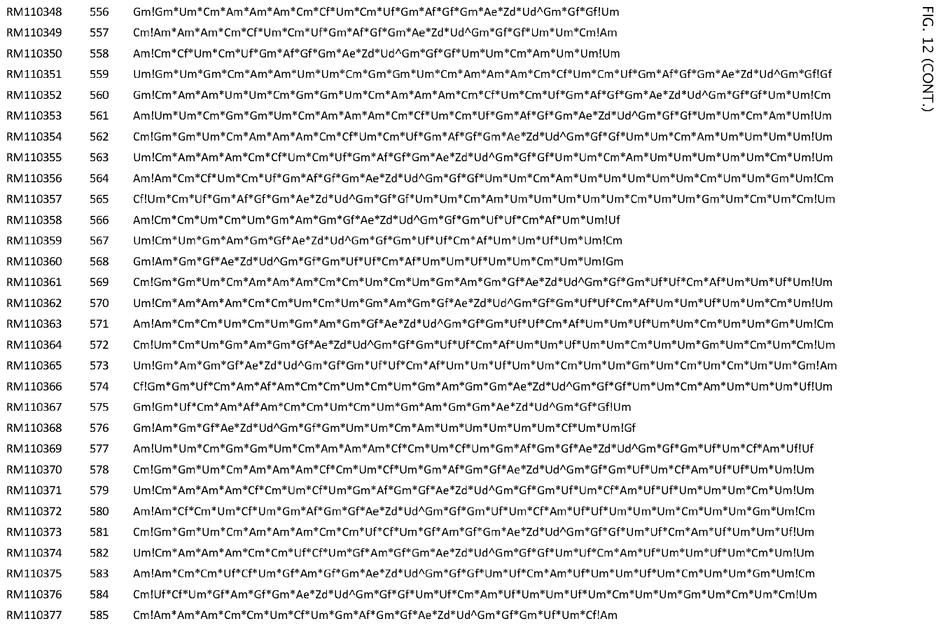

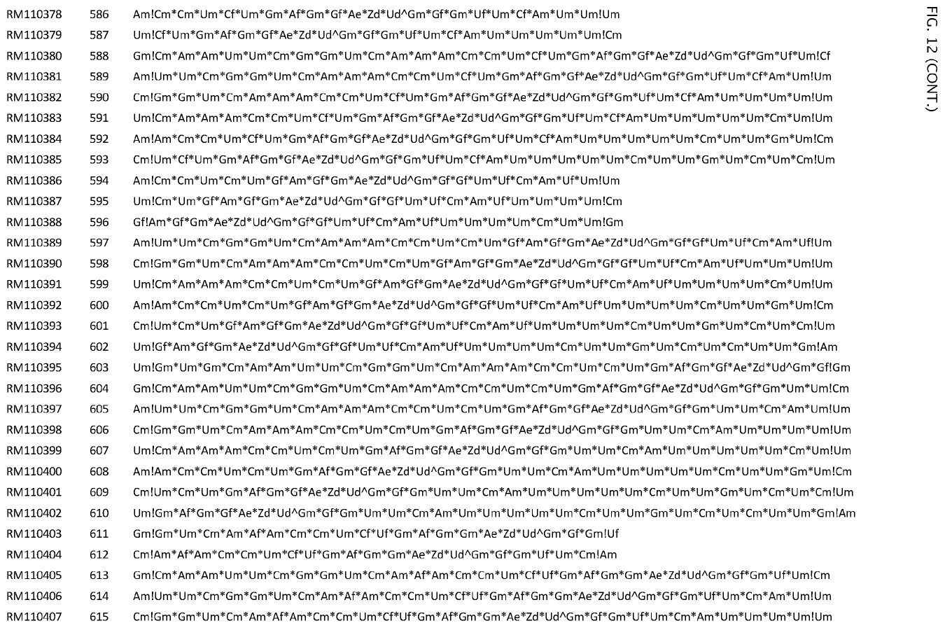

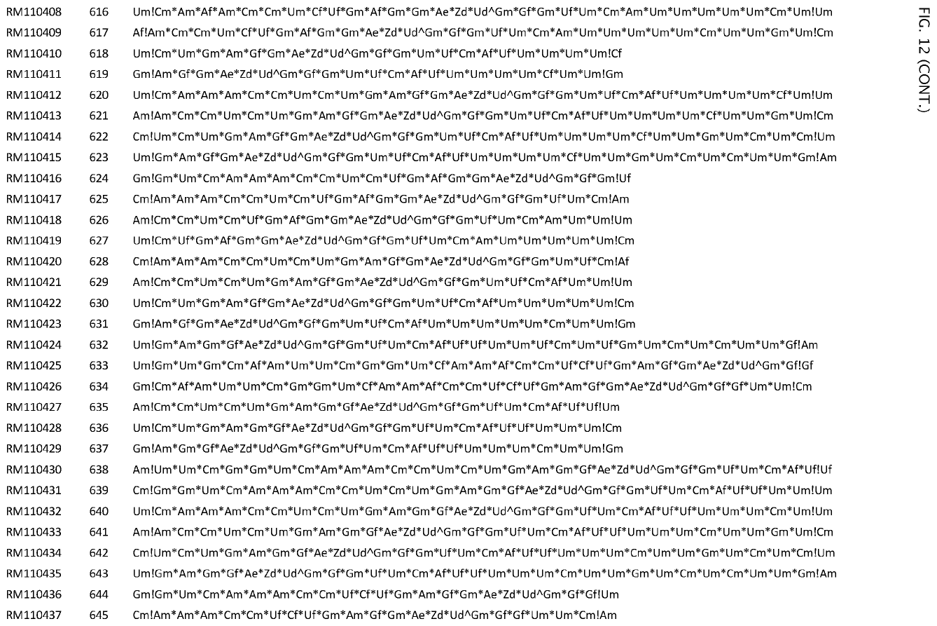

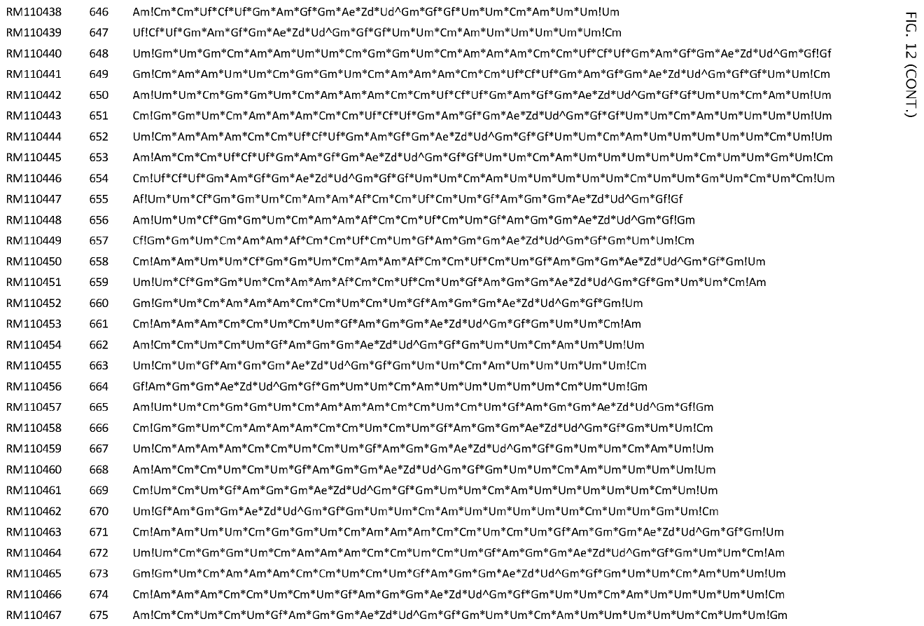

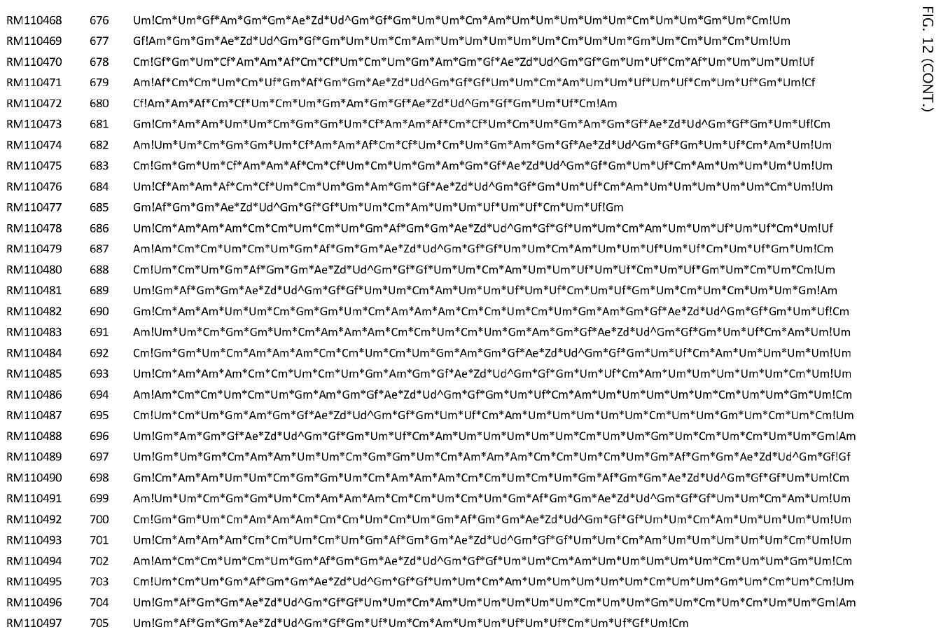

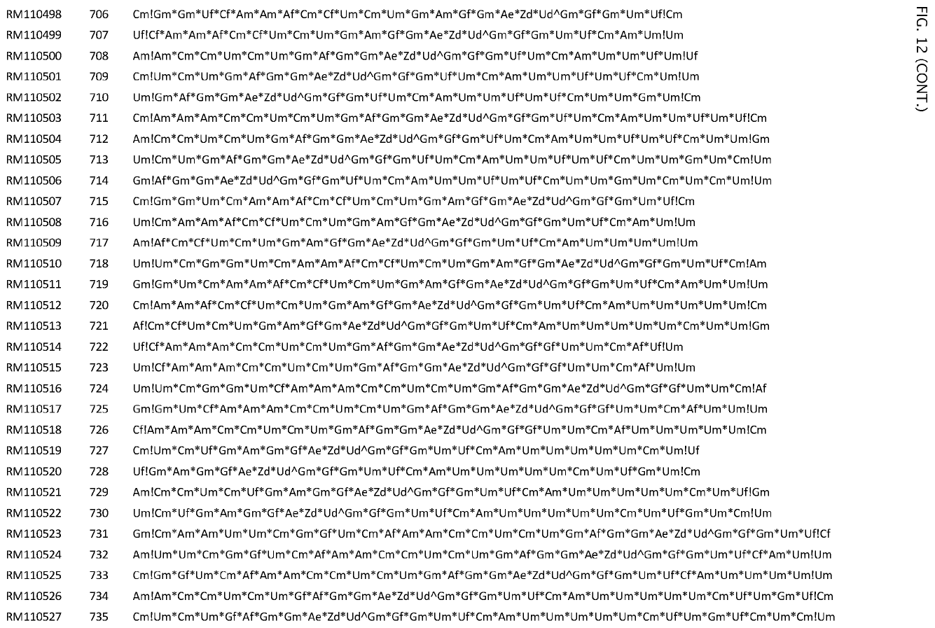

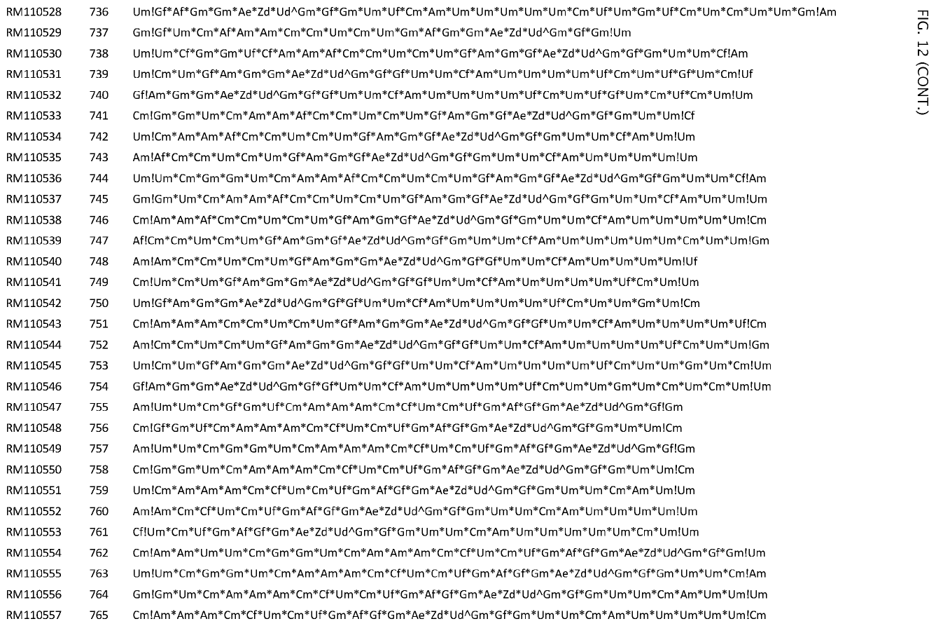

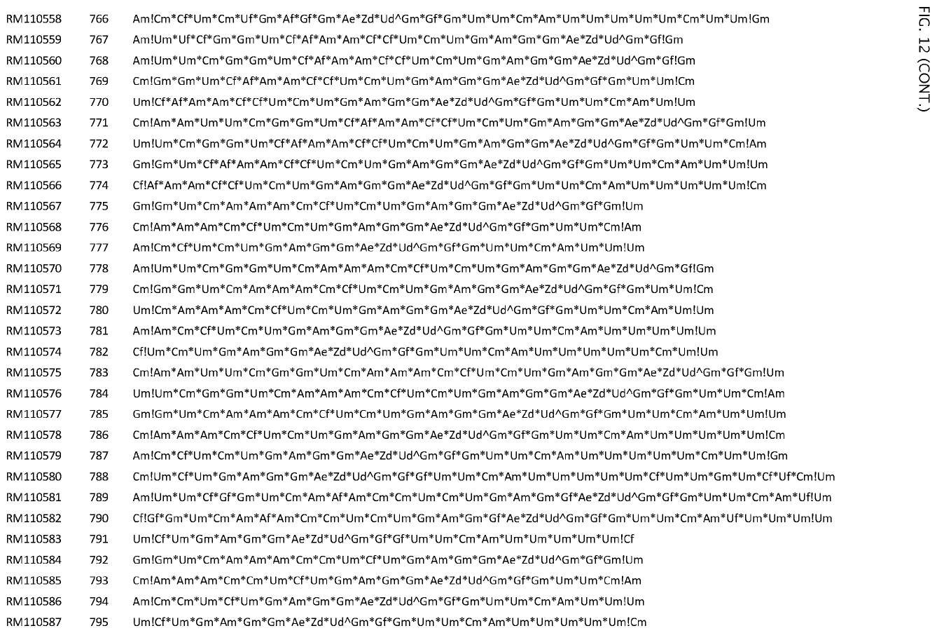

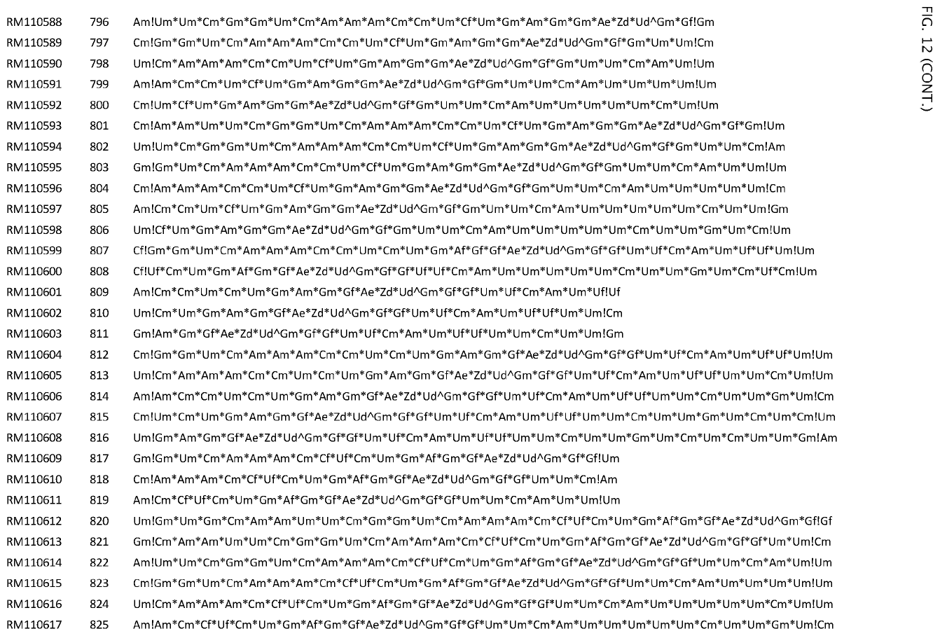

To investigate the potential of what has been disclosed herein and further varying the chemical modifications and lengths of the oligonucleotides to ultimately improve on the editing of B4GALT1 transcripts, an additional set of 960 EONs was designed that each or independently are tested in PHHs, PMHs, liver spheroids, and/or in vivo as outlined in the previous examples. These 960 EONs with their respective RM numbers and chemical modifications are provided in FIG. 12. From this set, as well as from the other designed EONs outlined intra, the best performing candidates are used either with or without a GalNAc moiety (and TEG linker) and formulated in an LNP formulation if needed. These best performing candidates are taken into further (pre-) clinical development to ultimately treat CVD in patients in need thereof.

### Example 10. Editing of Human B4GALT1 Transcripts in Primary Human Hepatocytes

Yet, another set of 22 EONs was designed that were tested together with some of the better performing editing-causing EONs from earlier screens:

$$B 4 GALT 1 - 05 = EON 05 = RM 4830 = SEQ ID NO : 23 ;$$

$$B 4 GALT 1 - 13 = EON 13 = RM 4838 = SEQ ID NO : 31 ;$$

$$B 4 GALT 1 - 69 = RM 107689 = SEQ ID NO : 1064 ; and$$

$$B 4 GALT 1 - 84 = RM 107704 = SEQ ID NO : 1068.$$

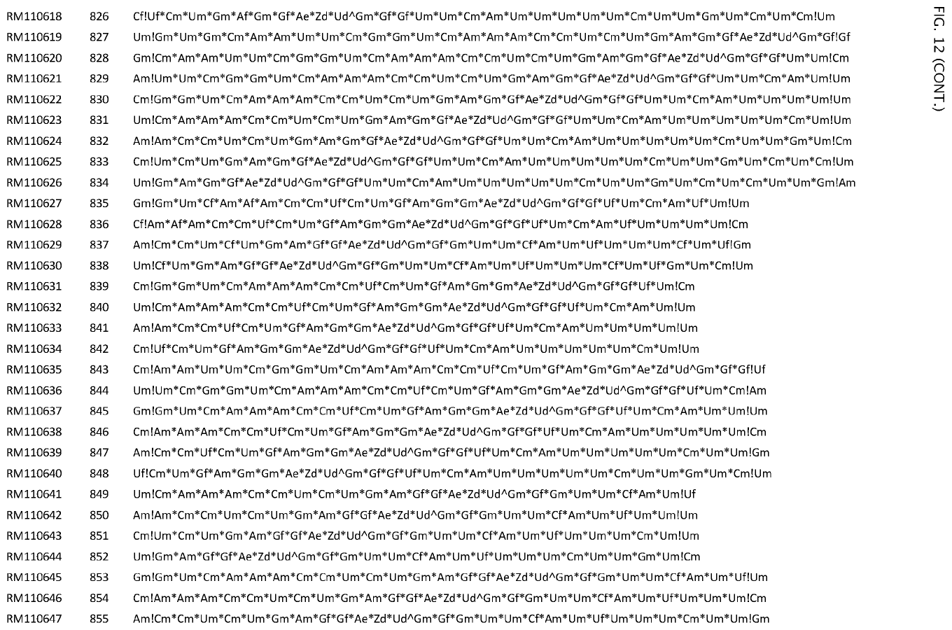

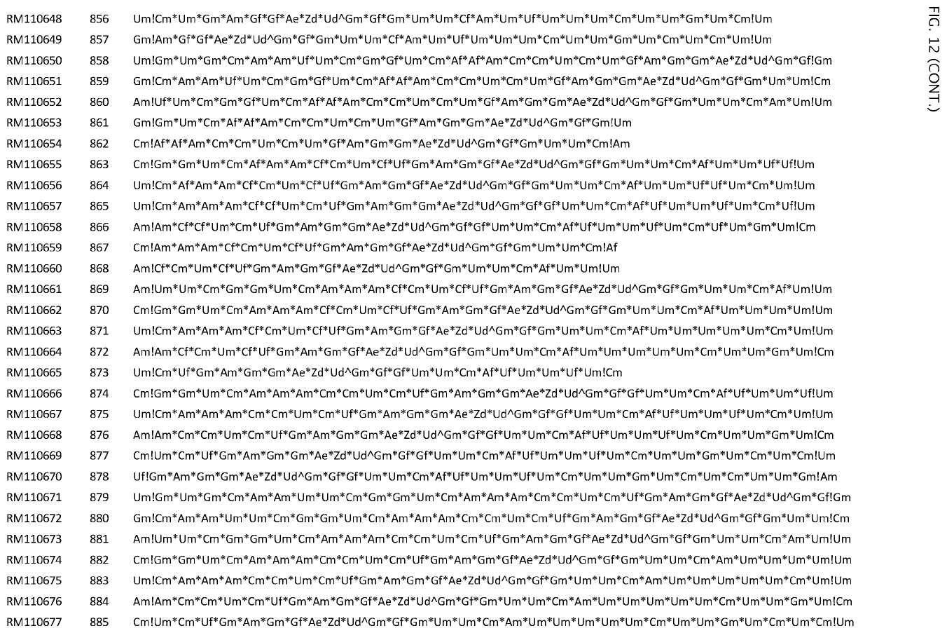

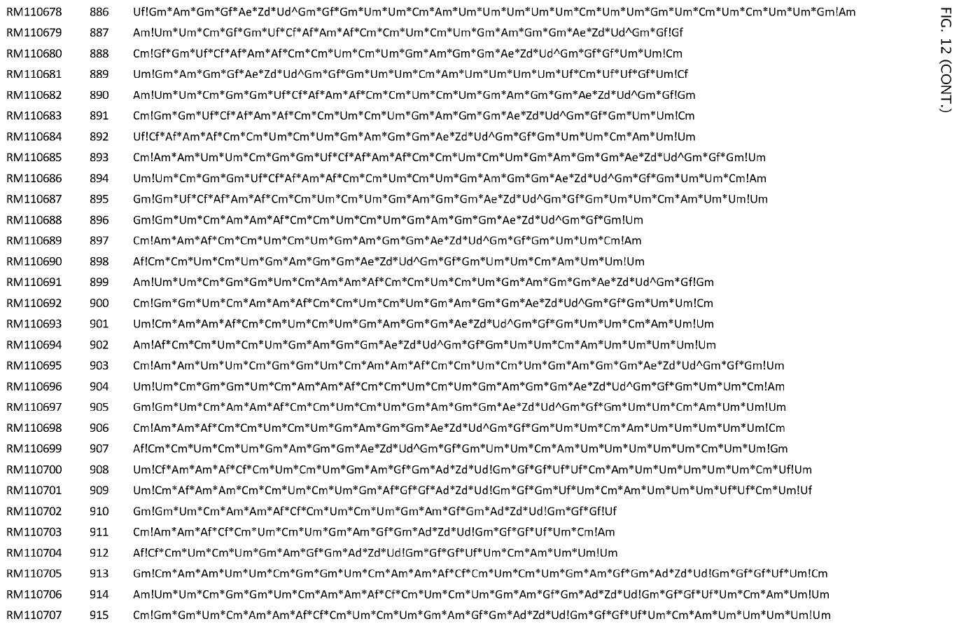

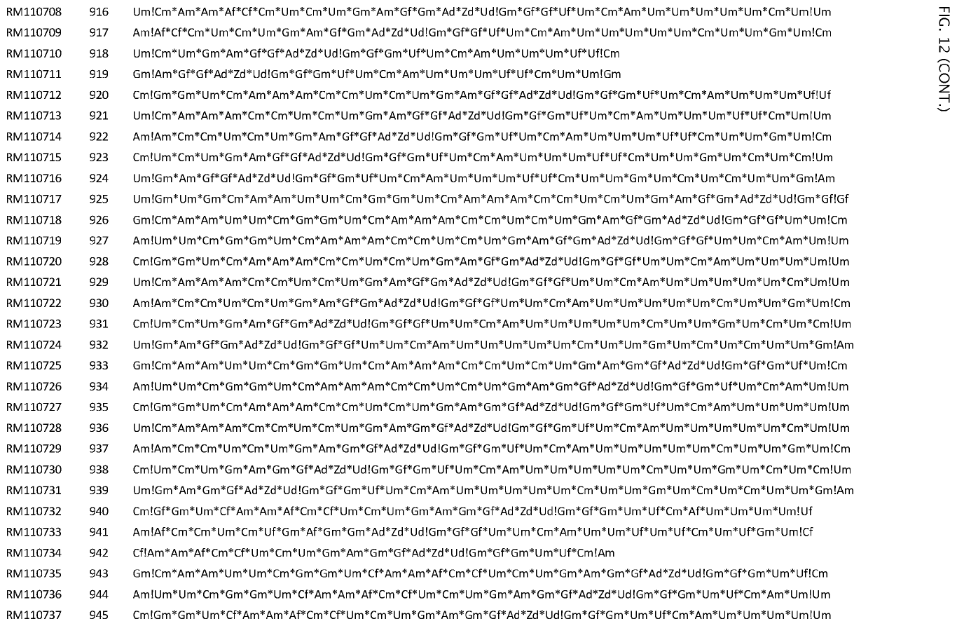

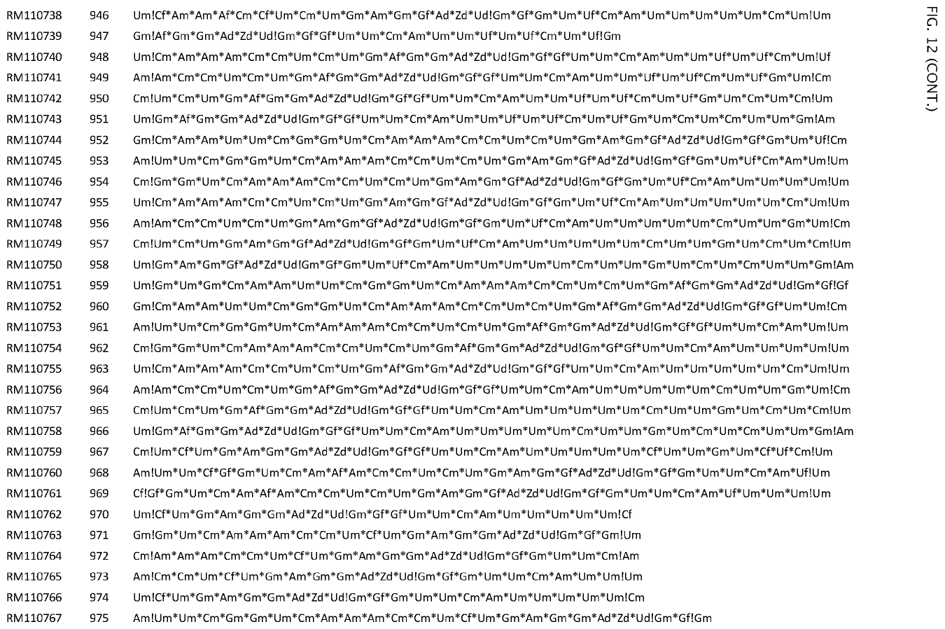

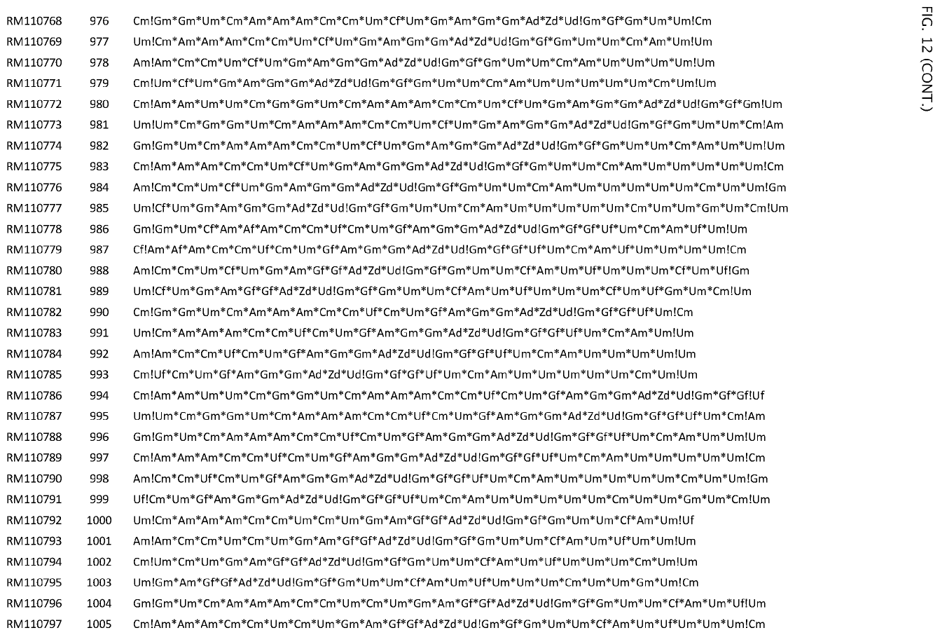

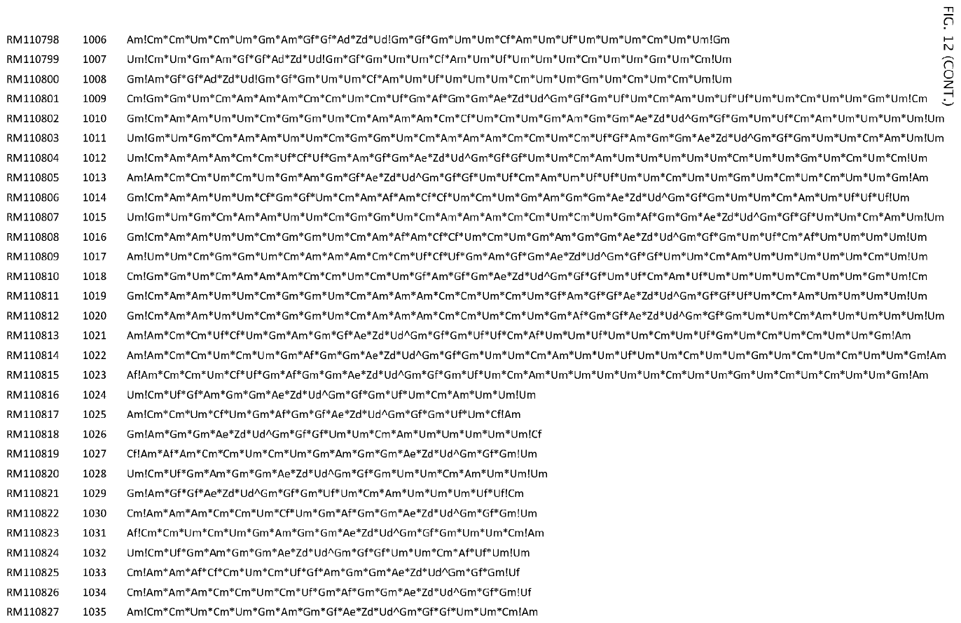

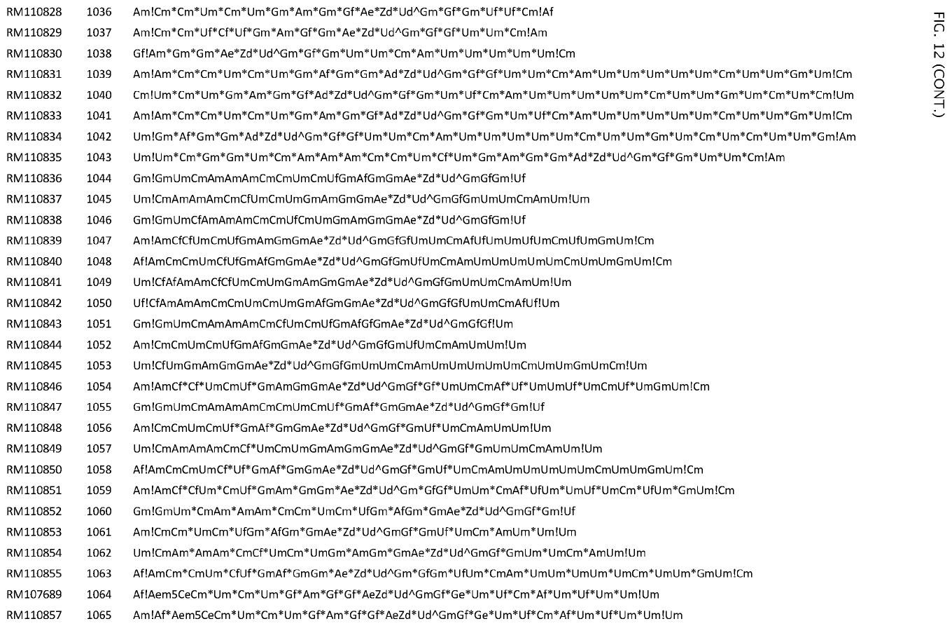

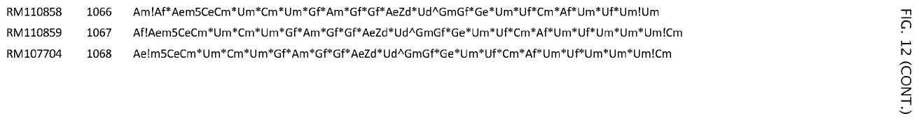

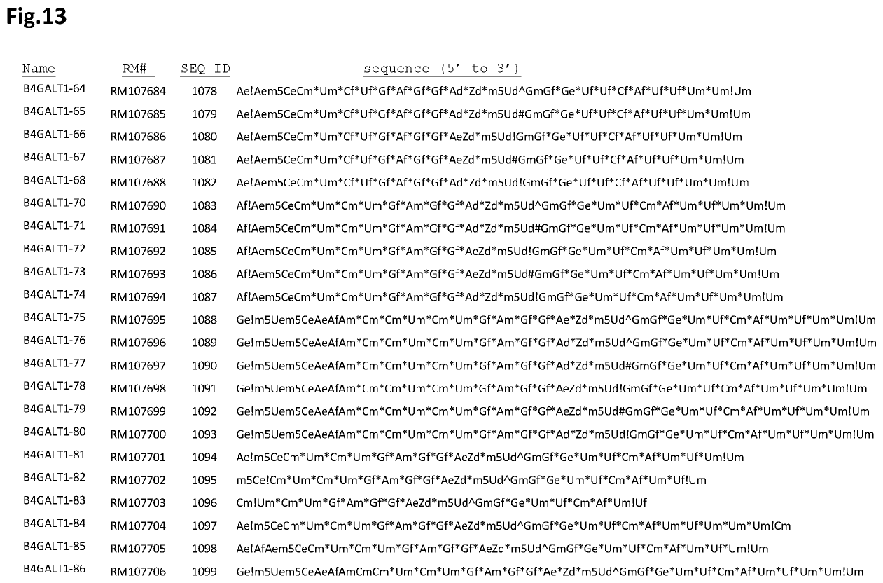

This new set of 22 EONs with their respective RM numbers and chemical modifications is given in FIG. 13.

Using the EONs, editing of the endogenous human B4GALT1 transcript was investigated in PHHs. A non-treated (NT) sample and a sample with saponin alone were taken along as negative controls. Cells were cultured, plated, and treated as indicated above in Example 5, except that the cells were co-treated with 0.5 UM AG1856 per well. RNA purification, cDNA generation and dPCRs were performed as outlined in Example 2, with the indicated primers and probes.

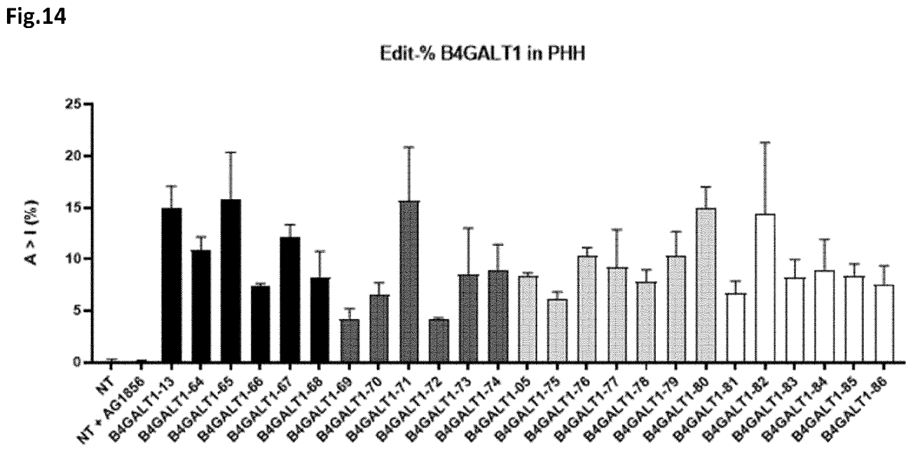

Results are depicted in FIG. 14, which shows that all the tested EONs provided relatively high levels of editing, with B4GALT1-13, B4GALT1-65 (SEQ ID NO: 1079), B4GALT1-71 (SEQ ID NO: 1084), B4GALT1-80 (SEQ ID NO: 1093), and B4GALT1-82 (SEQ ID NO: 1095) performing best.

### Example 11. Exon 5 Skipping in Human B4GALT1 Transcripts after EON Treatment

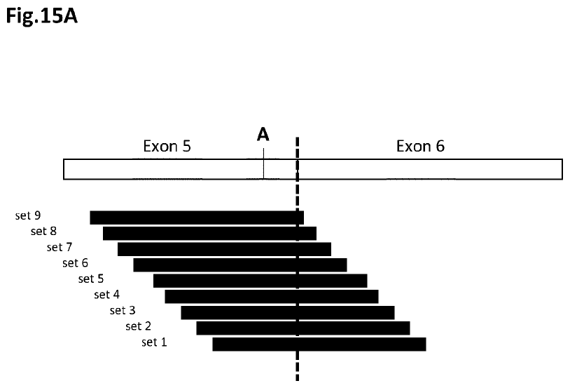

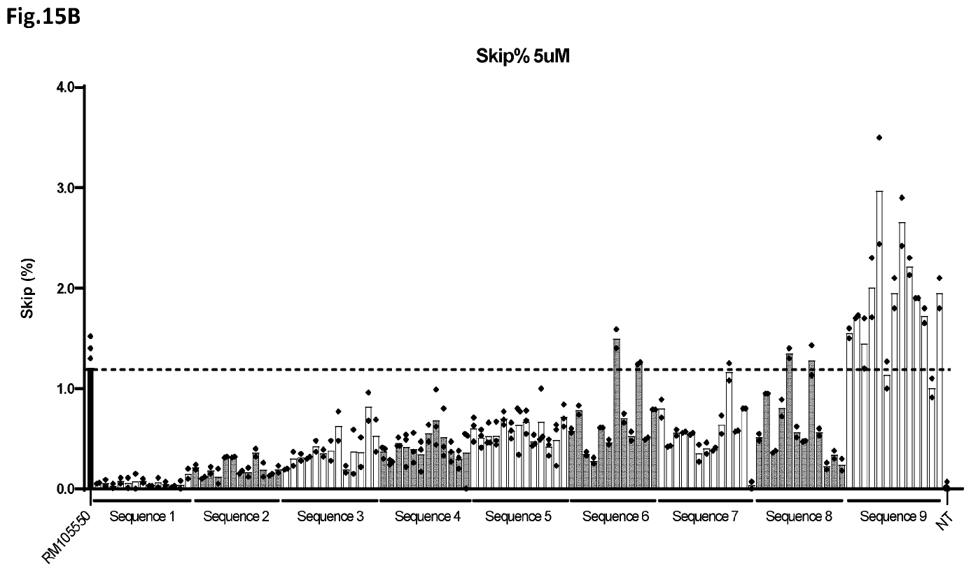

As discussed hereinabove, the aim of the EONs is to cause editing of the human B4GALT1 transcript by introducing an A>I deamination, thereby generating an N352S mutation (or ‘variant’) in the protein sequence. The target adenosine at position 1055 in the transcript is relatively close to the 3′ terminus of exon 5 in the pre-mRNA, and it was observed that some of the EONs as disclosed herein cause (besides triggering the desired RNA editing) also the skip of exon 5 from the pre-mRNA, which results in an out-of-frame transcript, which in turn also causes the downregulation of active B4GALT1. Hence, although splice modulation is often unwanted, in this case it may be seen as a side-effect (or an unexpected bonus effect), which may be beneficial in the treatment of CVD. Therefore, it was investigated to what extent the position of the complementary sequence of the EON with the target RNA would influence the skipping of exon 5. For this, a large set of EONs were designed, with 12 separate EONs of the same length but with different chemical modifications (including GalNac and a TEG linker) in 9 separate sets. Hence, 9×12 EONs were tested in their ability to cause exon skipping, from which it could potentially be determined how their position would relate to the efficiency in exon skipping. FIG. 15(A) shows the exon5/exon6 boundary and the relative position of the target A in exon 5. Below that schematic representation, the positions of the 9 sets of EONs are provided, in which it should be noted that these are represented schematically from 3′ to 5′ with the 5′ ends of the EONs extending into intron 5 such that it predominantly targets the pre-mRNA. The EONs were tested in PHH cells with 5×10⁴ cells per well in a 96-well format and incubated in a concentration of 5 μM EON and co-treated with 1 μM AG1856. RM105550 (=B4GALT1-13 with a GalNAc moiety but without a TEG linker; identical to RM106564; SEQ ID NO:1100) served as a control. After 72 hr cells were harvested and subsequently exon 5 skip was determined as described above. FIG. 15(B) shows the results obtained with all separate EONs. Below the graph the subsets are given. This result clearly shows that when the EON is complementary to a sequence that is relatively far away from the 3′ end of exon 5, more exon skipping is observed. However, when the complementarity ‘moves’ towards the exon5/intron5 boundary, skipping efficiency decreased significantly. This shows that the position of the EON and where it hybridizes to the target sequence not only influences the RNA editing, but also has an impact on the skipping of exon 5, which together may add to downregulating the function of the B4GALT1 protein, as desired and as discussed herein.

### Example 12. Editing of Human B4GALT1 Transcripts in Primary Human Hepatocytes Using EONs with Varying Position of the 2′-Fluor Modification

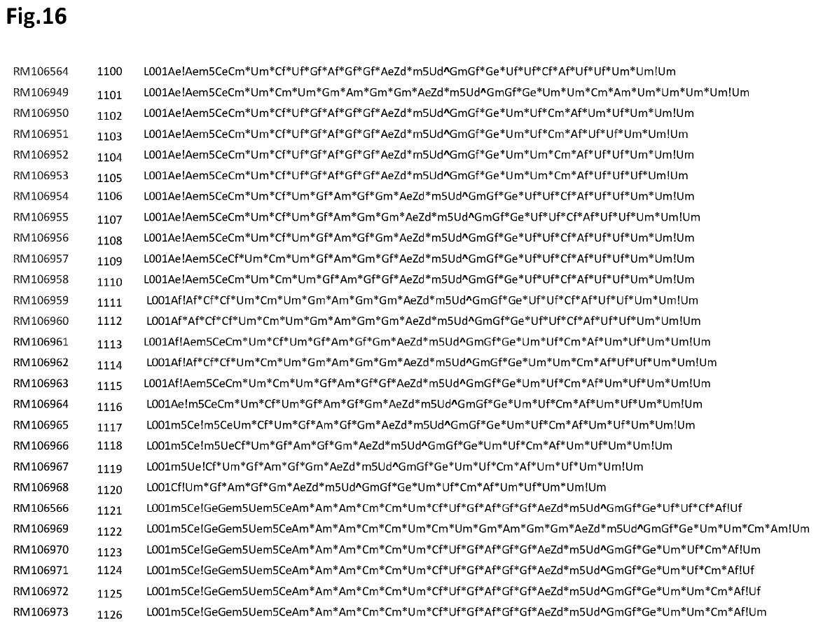

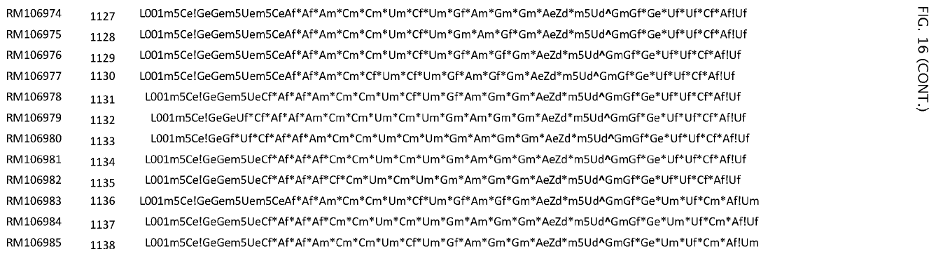

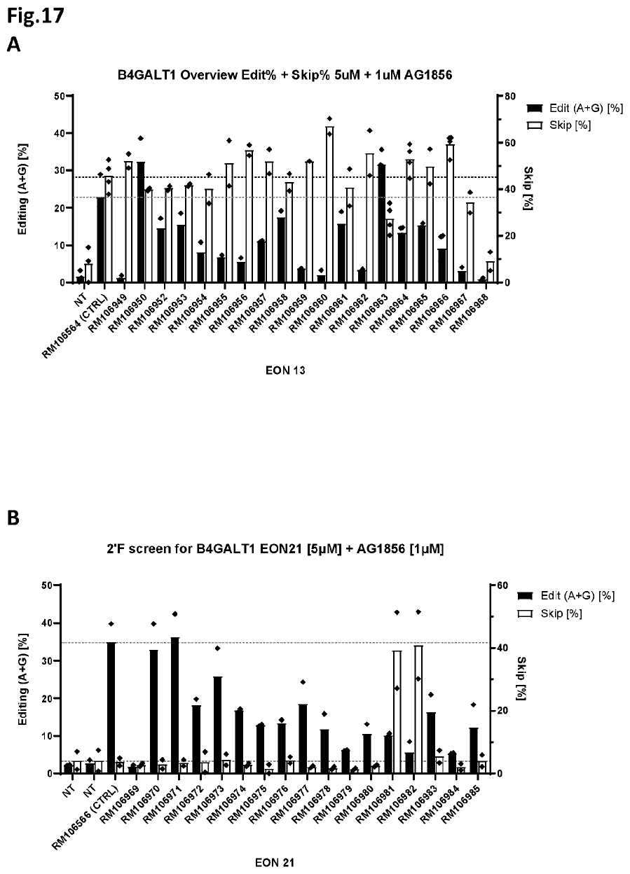

It was tested whether the positions of the 2′-F modifications present in the EON would influence the effect on RNA editing and/or on exon 5 skipping. For this, a set of 20 EONs was designed based on the sequence and modifications described for B4GALT1-13 (RM106564; SEQ ID NO: 1100, which is identical to RM105550 in FIG. 15). The different B4GALT1-13-based EONs and their 2′-F patterns are provided in FIG. 16 (SEQ ID NO: 1101 to 1120). This shows that RM 106949 (SEQ ID NO: 1101)-except for one position-completely lacks 2′-F modifications. Using these EONs, the same experiment as outlined in Example 11 was performed except that the purified RNA was used to determine exon 5 skip percentages, for cDNA generation and a dPCR to determine RNA editing of the target adenosine. FIG. 17(A) shows the editing percentages (black bars) and the percentage exon 5 skip (open bars) for each of the 20 EONs in comparison to B4GALT1-13 (RM106564; SEQ ID NO: 1100 (see also RM4838; SEQ ID NO:31)). Strikingly, while RM106564 gave an editing percentage of around 22%, and an exon skip percentage of 43%, RM106949 gave significant exon skip (>50%) but hardly any RNA editing, suggesting that the 2′-OMe modifications that are present instead of the 2′-F modifications hamper proper deamination by the ADAR enzyme for this target, while not interfering with splice modulation. Importantly, some EONs, such as RM106950 (SEQ ID NO: 1102) and RM106963 (SEQ ID NO:1115), showed editing percentages above 30%, while the exon 5 skipping was below 30%, indicating that the positions of the 2′-F modifications are important for obtaining the different effects. A similar experiment was performed with 17 EONs (SEQ ID NO: 1122 to 1138) that were based on the B4GALT1-21 EON (RM106566; SEQ ID NO: 1121 (see also RM4846; SEQ ID NO: 39)). The results of an identical experiment are shown in FIG. 17(B) and indicate that some EONs such as RM106970 (SEQ ID NO:1123) and RM 106971 (SEQ ID NO:1124) performed equally well as RM106566 in editing (up to 35%) and exon 5 skipping (around 2%).

1. An RNA editing oligonucleotide (EON) that can form a double-stranded complex with a region of a target RNA nucleic acid molecule in a human cell, wherein the double-stranded complex can recruit an endogenous ADAR enzyme naturally present in the cell, wherein the region comprises a target adenosine, wherein the nucleotide in the EON that is opposite the target adenosine is the orphan nucleotide, wherein the ADAR enzyme deaminates the target adenosine into an inosine, and wherein the target RNA nucleic acid molecule is a transcript molecule of the human β-1,4-galactosyl-transferase 1 (B4GALT1) gene.

2. An EON according to claim 1, wherein the B4GALT1 transcript molecule is a pre-mRNA or an mRNA molecule.

3. An EON according to claim 1 or 2, wherein the cell is a human liver cell, preferably a hepatocyte.

4. An EON according to any one of claims 1 to 3, wherein the target adenosine is at a position in the B4GALT1 transcript where a guanosine would encode a B4GALT1 protein variant that has a reduced enzymatic turnover rate.

5. An EON according to any one of claims 1 to 4, wherein the target adenosine is at position c. 1055A in the B4GALT1 transcript, and wherein the deamination results in a change from asparagine (N; Asn) to serine (S; Ser) at position 352 in the human wildtype B4GALT1 amino acid sequence.

6. An EON according to any one of claims 1 to 5, wherein at least one nucleotide comprises one or more non-naturally occurring chemical modifications in the ribose, linkage, or base moiety, with the proviso that the orphan nucleotide is not a cytidine comprising a 2′-OMe ribose substitution.

7. An EON according to claim 6, wherein the one or more modifications in the linkage is each independently selected from a phosphorothioate (PS), a phosphonoacetate, phosphorodithioate, a methylphosphonate (MP), a sulfonylphosphoramidate, or a PNdmi internucleoside linkage.

8. An EON according to claim 6 or 7, wherein the one or more modifications in the ribose moiety is a mono- or di-substitution at the 2′, 3′ and/or 5′ position of the ribose, each independently selected from the group consisting of: —OH; —F; substituted or unsubstituted, linear or branched lower (C₁-C₁₀) alkyl, alkenyl, alkynyl, alkaryl, allyl, or aralkyl, that may be interrupted by one or more heteroatoms; —O-, S-, or N-alkyl; —O-, S-, or N-alkenyl; —O-, S-, or N-alkynyl; —O-, S-, or N-allyl; —O-alkyl-O-alkyl; -methoxy; -aminopropoxy; -methoxyethoxy; -dimethylamino oxyethoxy; and -dimethylaminoethoxyethoxy.

9. An EON according to any one of claims 1 to 8, wherein the EON comprises or consists of an EON selected from the group provided in SEQ ID NO:3 to 42, 59 to 1069, and 1078 to 1190, preferably selected from the group consisting of SEQ ID NO:23, 19, 31, 27, 35, 39, 69, 70, 71, 72, 73, 93, 94, 95, 1079, 1084, 1093, 1095, 1100, 1102, 1115, 1121, 1123, 1124, and 1139 to 1190.

10. An EON according to any one of claims 1 to 9, wherein the orphan nucleotide is a deoxynucleotide carrying a 6-amino-5-nitro-3-yl-2 (1H)-pyridone nucleobase or an iso-uracil nucleobase.

11. A vector, preferably a viral vector, more preferably an adeno-associated virus (AAV) vector, comprising a nucleic acid molecule encoding an EON according to any one of claims 1 to 5.

12. A nanoparticle delivery vehicle formulation that comprises an EON according to any one of claims 1 to 10.

13. A nanoparticle delivery vehicle formulation according to claim 12, wherein the nanoparticle delivery vehicle is a Lipid Nanoparticle (LNP).

14. A pharmaceutical composition comprising an EON according to any one of claims 1 to 10, a vector according to claim 11, or a nanoparticle delivery vehicle formulation according to claim 12 or 13, and a pharmaceutically acceptable carrier.

15. An EON according to claims 1 to 10, a vector according to claim 11, a nanoparticle delivery vehicle formulation according to claim 12 or 13, or a pharmaceutical composition according to claim 14, for use in the treatment of cardiovascular disease (CVD).

16. Use of an EON according to claims 1 to 10, a vector according to claim 11, a nanoparticle delivery vehicle formulation according to claim 12 or 13, or a pharmaceutical composition according to claim 14, in the manufacture of a medicament for the treatment of CVD.

17. An in vitro, ex vivo, or in vivo method of editing a B4GALT1 transcript molecule, the method comprising contacting the B4GALT1 transcript molecule, or a part thereof, with an AON according to any one of claims 1 to 10, thereby editing the B4GALT1 transcript molecule.

18. A method of treating, slowing down, or ameliorating CVD in a patient in need thereof, the method comprising contacting a B4GALT1 transcript molecule in a cell of the subject with an EON according to any one of claims 1 to 10, thereby treating the patient.

19. A method for the deamination of a target adenosine in an B4GALT1 transcript molecule in a cell, the method comprising the steps of:
(i) providing the cell with an EON according to any one of claims 1 to 10;
(ii) allowing uptake by the cell of the EON;
(iii) allowing annealing of the EON to the B4GALT1 transcript molecule;
(iv) allowing an endogenous ADAR enzyme that is naturally present in the cell to deaminate the target adenosine in the target RNA molecule to an inosine; and optionally
(v) identifying the presence of the inosine in the target RNA molecule.

20. A method according to claim 19, wherein the cell is a human cell, preferably a liver cell, more preferably a hepatocyte, wherein the target adenosine is at position c. 1055A in the B4GALT1 transcript, and wherein the deamination results in a change from asparagine (N; Asn) to serine (S; Ser) at position 352 in the human wildtype B4GALT1 amino acid sequence.

21. A method according to claim 19 or 20, wherein step (v) comprises:
a) sequencing the B4GALT1 pre-mRNA or mRNA molecule, or a cDNA derived thereof;
b) assessing the presence of a 352Ser B4GALT1 protein variant; or
c) using a functional read-out, preferably assessing a reduction rate of UDP-Gal, or assessing glycosylation levels of transferrin in serum.
